(19) United States
(12) Patent Application Publication
(10) Pub. No.: US 2026/0028650 A1
(43) Pub. Date: Jan. 29, 2026

(54) METHOD TO PRODUCE ALCOHOL AND ALCOHOL PRECURSORS USING GENETICALLY MODIFIED BACTERIA

(71) Applicant: Chemical Evolution Ltd., Calgary (CA)

(72) Inventors: Dustin LILLICO, Calgary (CA); Trevor RANDALL, Calgary (CA); Alejandra ENRIQUEZ, Calgary (CA); Markus WEISSENBERGER, Calgary (CA)

(21) Appl. No.: 19/038,654

(22) Filed: Jan. 27, 2025

(30) Foreign Application Priority Data
Jul. 23, 2024 (CA) NOT YET KNOWN

(51) Int. Cl. C12P 7/10; C08H 8/00; C12N 9/42

(52) U.S. Cl. CPC C12P 7/10; C08H 8/00; C12N 9/2437; C12Y 302/01021; C12Y 302/01091

(57) ABSTRACT

A method of producing an alcohol or alcohol precursor from a cellulosic material and a genetically modified live ethanologenic organism, wherein the method comprises the steps of:
exposing said genetically modified live ethanologenic organism to a culture media with a pH of between 2 and 9 thereby creating an incubation mixture;
exposing said incubation mixture to a source of cellulose;
incubating said genetically modified live ethanologenic organism in said incubation mixture with said source of cellulose under aerobic and/or anaerobic conditions at a temperature ranging from 0° C. to 60° C. for a first period of time sufficient for the degradation of cellulose, thereby creating a degraded cellulose mixture;
optionally, incubating said genetically modified live ethanologenic organism in said degraded cellulose mixture under aerobic and/or anaerobic conditions at a temperature ranging from 0° C. to 60° C. for a second period of time sufficient for the production of said alcohol or alcohol precursor; and
optionally, recovering said alcohol or alcohol precursor from the cells and/or spent culture media.
   wherein said genetically modified live ethanologenic organism comprises at least one of the following polynucleotide sequences in its genome:
i. at least one endoglucanase (cen-like) polynucleotide sequence selected from the group consisting of an endoglucanase A-like (cenA-like) polynucleotide sequence, an endoglucanase B-like (cenB-like) polynucleotide sequence and an endoglucanase C-like (cenC-like) polynucleotide sequence;
ii. an exoglucanase (cex-like) polynucleotide sequence; and
iii. a β-glucosidase 1 (bgl1) polynucleotide sequence;
   wherein said source of cellulose has a lignin content of at most 1 wt. % and a hemicellulose content of at most 15 wt. %, and wherein said live ethanologenic organism belongs to a genus is selected from the group consisting of: Aspergillus, Mucor, Zymomonas, Escherichia, Clostridia, Bacillus, and Pseudomonas.

### REFERENCE TO AN ELECTRONIC SEQUENCE LISTING

The contents of the electronic sequence listing (2025-08-06_SequenceListing_ST26_23156-204US1.xml; Size: 286,736 bytes; and Date of Creation: Aug. 5, 2025) is herein incorporated by reference in its entirety.

### FIELD OF THE INVENTION

The present invention relates to a method to convert a lignocellulosic material into an alcohol and/or alcohol precursors through genetically manipulated bacteria using naturally present or added metabolic pathways.

### BACKGROUND OF THE INVENTION

Biofuel is increasingly becoming a necessity in order to wean off the human consumption of fossil fuels in aspects of everyday life, transport and home heating being the largest two industries of focus. As an alternative energy source to oil and coal, the main feedstock for bioethanol production is starch which can yield its monosaccharide much more readily than cellulose. This is due to the difference in structure as starch contains glucose molecules connected through β-1,4 linkages and cellulose comprises of glucose molecules attached through β-1,4 linkages. The β-1,4 linkages allow for crystallization of the cellulose, leading to a more rigid structure, which is more difficult to break down via physical, chemical, or biological processes.

The limitation that comes from solely concentrating the bioethanol on extracting the sugars from starches prevents the utilization of the larger portion of biomass which comes in the form of lignocellulosic biomass (contains lignin, cellulose, and hemicellulose) present in almost every plant on earth. A delignification reaction allows the recovery of cellulose from those lignocellulosic plants. Further degradation of the cellulose generates cellobiose and/or glucose which can be utilized for the production of ethanol using various biology-based processes.

Seen as a sustainable alternative to gasoline and with the goal of alleviating many countries' dependence on foreign oil, the bioethanol industry is still hampered by its dependence on corn or sugar cane as main sources of fuel, as they are both rich in starch. It is estimated that about 45% of all corn production in the U.S. is directed to ethanol fuel production. This is a situation which has disastrous consequences when the prices of gasoline decline significantly low making corn-based bioethanol unsustainable on a price viewpoint.

Across the world, many other large ethanol-producing countries, including China and Brazil, have shown some struggles in ethanol production from biomass as many companies are carrying large debts from the implementation of such processes in conjunction with large processing plants having to be shut down or decrease production.

In Asia, palm oil prices have recently increased to their highest levels in years, which, in turn, will hamper the ability of Indonesia and Malaysia to produce local biofuel. Oil palm trunk is a valuable and plentiful resource in those countries to generate biofuels and biochemicals. Oil palm trunk contains a large amount of starch which is more readily solubilized in water, compared to cellulose. The starch can then be heated and hydrolyzed to glucose by amylolytic enzymes without pre-treatment. However, the conventional Oil palm trunk treatment requires high capital and operational costs and is therefore prohibitive to market entry. Moreover, the treatment carries a high probability of microbial contamination during starch processing which significantly reduces purity and poses additional complications in refining procedures.

In Europe, the biofuel industry (both biodiesel and bioethanol production) depends heavily on food-based feedstock like virgin vegetable oils (i.e., rapeseed, palm oil, soy) for biodiesel and corn, wheat, and sugar beet for bioethanol. Simultaneously, concerns have been raised that making fuel out of crops displaces other crops and can inflate food prices. These concerns are leading to policy changes that incentivize a shift away from food-based biofuels.

The pivot from starches to cellulose for the production of glucose is preferable as it will cease to use a food source to generate glucose. However, the costs to do so are currently prohibitive. Cellulosic ethanol as it is called relies on the non-food component of a plant to be used to generate ethanol. This would allow the replacement of the current more widespread approach of making bioethanol by using corn or sugarcane. The diversity and abundance of these types of cellulose-rich plants would allow for food resources to remain largely intact and capitalize on the waste generated from these food resources (such as cornstalk and stover) to generate ethanol. Other cellulose sources such as straws, algae and even trees fall under cellulose-rich biomass sources which can be used in generating ethanol if a commercially viable process is developed.

The reason why starches are preferred to cellulose-rich sources to generate ethanol is that the extraction of glucose from cellulose is substantially more difficult and resource intensive. To better understand the conditions associated with this increased difficulty, it is worthwhile describing the structural similarities and differences between starch and cellulose.

Cellulose and starch are polymers which have the same repeat units of glucose. However, the differences between starch and cellulose can be seen in the way the repeating glucose monomers are connected to one another. In starch, the glucose monomers are oriented in the same direction. In cellulose, each successive glucose monomer is rotated 180 degrees in respect to the previous glucose monomer. This, in turn, ensures that the bonds between each monomeric glucose differs between starch and cellulose. In starch, the bonds (otherwise known as links between the repeat units) are referred to as α-1,4 linkages, in cellulose these bonds are referred to as β-1,4 linkages (see FIG. 1).

The difference between these bonds impacts the chemical characteristics of starch and cellulose. Starch can dissolve in warm water while cellulose does not. Starch can be digested by humans, cellulose cannot. In general, starch is structurally weaker than cellulose partly due to its geometrical make-up which is less crystalline than cellulose. Starch is, at its core, a method for plants to store energy due to the reversibility of the as α-1,4 linkage, therefore extracting sugars from starch is much easier than to do so from cellulose as the latter's core function is to provide structural support.

As the main component of lignocellulosic biomass, cellulose is a biopolymer consisting of many glucose units connected through β-1,4-glycosidic bonds (see FIG. 1). Glucose has two isomers: α-glucose (present in starches as branched polymers) and β-glucose (present in cellulose connected via a β-1,4-glycosidic bond with one β-glucose monomer rotated by 180 degrees relative to its neighbour). A cellulose molecule can comprise between hundreds to thousands of glucose units. Since the cellulose molecules are linear, due in part to intermolecular hydrogen bonding, neighboring cellulose molecules can be very closely packed, partially crystallized and, in turn, provide the structural strength necessary to support plants.

### Hydrolysis of Cellulose

The hydrolysis of cellulose is the rate limiting step in the conversion of cellulose into biofuel. The processes currently using cellulose as a starting material for bioethanol production require the conversion of cellulose into cellobiose, further processing to generate glucose, and the final generation of ethanol. The fermentation of glucose using ethanologenic organisms is what leads to the production of ethanol. While that last step in biofuel production has been mastered for some time, the rate limiting step of cellulose hydrolysis is the most crucial one which hinders a wider acceptance of biofuels. The difficulty in overcoming this conversion of cellulose into glucose lies with the fact that cellulose has a crystalline structure which renders its conversion to glucose quite difficult because of the close packing of multiple cellulose polymers. This close packing imparts cellulose its inherent stability under a variety of chemical conditions. For example, cellulose polymers are generally insoluble in water, as well as a number of organic solvents. Furthermore, cellulose is also generally insoluble when exposed to weak acids or bases.

In general, there are two main approaches to hydrolyze cellulose: chemical and enzymatic. The chemical method resorts to the use of concentrated strong acids to hydrolyze cellulose under conditions of high temperature and pressure. Many different types of acids, such as HCl and H₂SO₄, have been used in the past to achieve this. The use of one of these acids usually results in at least one of the following drawbacks: corrosion of the reaction vessel, difficulty of disposing of the discharged reactants, and others. The biofuel industry is generally reticent to use chemically hydrolyzed cellulose because of the presence of toxic by-products in the resulting glucose. These by-products, if introduced in the fermentation step, will negatively affect the delicate balance of the fermenting yeast.

### Cost of Enzymatic Hydrolysis

It is known that the costs to extract biofuel from cellulose are higher than when doing so from starch. It is estimated that, on average, depending on location and availability of biomass, the cost for cellulose conversion is about 50% more that starch conversion to glucose. This means that there currently is a clear barrier to producers for using cellulose rather than corn or other starch resources to generate glucose from biomass.

It is generally understood that roughly half of the total cost of producing biofuel from cellulose stems from the price of the enzymes (cellulases and hemicellulases). The generation of enzymes for enzymatic hydrolysis of cellulose is a time-consuming process and large volumes of enzyme are required to render the process commercially viable. One possible approach is to improve the rate of the hydrolysis reaction which, in turn, would result in a decrease in the overall cost of the process.

The enzymatic approach to hydrolyzing cellulose uses enzymes to carry out the hydrolysis reaction. Enzymes, such as cellulases (comprising endo-1,4-β-glucanases; exo-1,4-β-glucanases; and β-glucosidases) are used for the conversion of cellulose into glucose, however each requires extensive controls in place to maximize the reaction rates the enzymatic approach is expected to provide. Conditions such as temperature, pH, salinity; concentration of substrate and product are all factors that may affect enzyme activity with even small deviations of these parameters from the enzyme's optimal conditions resulting in loss of function. Overall, with many controls needing to be taken into consideration with the enzymatic hydrolysis of cellulose along with strict enzyme-specific functional conditions, such a method can render the process cost prohibitive in some cases and/or limit their implementation.

The enzymatic hydrolysis of cellulose is, as seen from the above, limited by the structure of cellulose itself but also by the approaches taken to degrade it into a biofuel. The production of a robust, low-cost process from cellulose has not yet been achieved. In this sense, genetically modified organisms have been employed for part of the entirety of the cellulose to ethanol pathway.

U.S. Pat. No. 4,496,656A describes a process for production of cellulase according to the present invention thus comprises culturing a cellulase-producing microorganism belonging to Cellulomonas uda CB4 in a cellulose-containing medium and recovering the cellulase produced from the culture broth. According to the present invention, because the bacteria belonging to Cellulomonas uda CB4 is capable of producing a cellulase having a high activity; not found in the reports of the prior art, in a culture medium, it is possible to produce a cellulase having a high crystalline cellulose decomposing activity comparable to those produced from a mold within a short cultivation period of two days.

In the paper titled “Expression of a cellulase gene in Zymomonas mobilis” by Misawa et al. (J. Biotechnology, 1988, 7 (3), 167-177), it was reported that a cellulase (CMCase) gene of Cellulomonas uda CB4 was introduced into Zymomonas mobilis NRRL B-14023 on pZA22, a cloning vector for Zymomonas, by conjugal transfer. Z. mobilis carrying this gene synthesized cellulase immunologically identical with that of C. uda CB4, by transcriptional read-through from the promotor of the chloramphenicol resistance (chloramphenicol acetyltransferase) gene within pZA22. A strong promotor containing a translation initiation signal was obtained from Z. mobilis NRRL B-14023 chromosomal DNA. By gene fusion between this Zymomonas promotor fragment and the truncated cellulase gene, the activity of cellulase synthesized in Z. mobilis reached 0.78 units per ml culture, six-fold higher than that by transcriptional read-through from the promotor of the chloramphenicol resistance gene.

In the paper titled “Expression of an endoglucanase gene of Pseudomonas fluorescens var. cellulosa in Zymomonas mobilis” by Lejeune et al. (FEMS Microbiology Letters, 1988, 49 (3), 363-366), the authors reported that by using a broad host-range, mobilizable plasmid vector, the endoglucanase gene eglX from Pseudomonas fluorescens var. cellulosa was cloned and expressed in the bacterial ethanologen Zymomonas mobilis. The enzyme was intracellular in this new host. It was produced throughout the growth phase and was not repressed by glucose. We postulate that transcription of the eglX gene was initiated from a promoter of the vector in Z. mobilis.

In the paper titled “Heterologous Expression and Extracellular Secretion of Cellulolytic Enzymes by Zymomonas mobilis” by Linger et al. (Appl Environ Microbiol. 2010 Oct; 76(19): 6360-6369), the authors reported that by using a technique known as consolidated bioprocessing (CBP), the initial steps toward achieving a single organism to convert pretreated lignocellulosic biomass to ethanol in the fermentation host Zymomonas mobilis were investigated. This was achieved by expressing heterologous cellulases and subsequently examining the potential to secrete these cellulases extracellularly. Numerous strains of Z. mobilis were found to possess endogenous extracellular activities against carboxymethyl cellulose, suggesting that this microorganism may harbor a favorable environment for the production of additional cellulolytic enzymes. The heterologous expression of two cellulolytic enzymes. E1 and GH12 from Acidothermus cellulolyticus, was examined. Both proteins were successfully expressed as soluble. active enzymes in Z. mobilis although to different levels. While the E1 enzyme was less abundantly expressed, the GH12 enzyme comprised as much as 4.6% of the total cell protein. Additionally, fusing predicted secretion signals native to Z. mobilis to the N termini of E1 and GH12 was found to direct the extracellular secretion of significant levels of active E1 and GH12 enzymes. The subcellular localization of the intracellular pools of cellulases revealed that a significant portion of both the E1 and GH12 secretion constructs resided in the periplasmic space. Our results strongly suggest that Z. mobilis is capable of supporting the expression and secretion of high levels of cellulases relevant to biofuel production, thereby serving as a foundation for developing Z. mobilis into a CBP platform organism.

In the paper titled “Evaluation of Cellulase Production by Zymomonas mobilis” by Todhanakasem and Jittjang (Bioresources, 2017, 12(1). 1165-1178), the authors report the use of Z. mobilis as a potential microbe for consolidated bioprocessing to convert lignocellulosic biomass to fermentable sugars while at the same time producing ethanol. To achieve this goal. Z. mobilis must be evaluated for the production of cellulolytic enzyme. This work reports on the potential of intracellular and extracellular crude extracts from Z. mobilis ZM4 and TISTR 551 to hydrolyze various cellulosic materials including carboxymethylcellulose (CMC), delignified rice bran, microcrystalline cellulose, and filter paper. Crude intracellular extracts from ZM4 and TISTR 551 showed high endoglucanase activity with CMC substrates at an optimal pH of 6 to 7 and temperature range of 30 to 40° C. The endoglucanase activity from the crude extracts was significantly higher than the exoglucanase activity. Of the high crystalline celluloses substrates tested, the best results were obtained for the hydrolysis of delignified rice bran by crude intracellular enzyme extracts of Z. mobilis TISTR 551.

In the paper by Vasan et al. titled “Cellulosic ethanol production by Zymomonas mobilis harboring an endoglucanase gene from Enterobacter cloacae” (Bioresource Technol. 2011, 102 (3), 2585), the authors report the use of a 2.25-kb fragment conferring cellulase activity from Enterobacter cloacae (isolated from the gut of the wood feeding termite, Heterotermes indicola), cloned in Escherichia coli. The cloned fragment contained a 1083-bp ORF which could encode a protein belonging to glycosyl hydrolase family 8. The cellulase gene was introduced into Zymomonas mobilis strain Microbial Type Culture Collection centre (MTCC) on a plasmid and 0.134 filter paper activity unit (FPU)/ml units of cellulase activity was observed with the recombinant bacterium. Using carboxymethyl cellulose and 4% NaOH pretreated bagasse as substrates, the recombinant strain produced 5.5% and 4% (v/v) ethanol respectively, which was threefold higher than the amount obtained with the original E. cloacae isolate. The recombinant Z. mobilis strain could be improved further by simultaneous expression of cellulase cocktails before utilizing it for industrial level ethanol production.

In the paper titled “Direct ethanol production from cellulosic materials by Zymobacter palmae carrying Cellulomonas endoglucanase and Ruminococcus β-glucosidase genes”, the authors disclose modifications that conferred the ability to ferment cellulosic materials directly on Zymobacter palmae by co-expressing foreign endoglucanase and β-glucosidase genes. The six genes encoding the cellulolytic enzymes (CenA, CenB, CenD, CbhA, CbhB, and Cex) from Cellulomonas fimi were introduced and expressed in Z. palmae. Of these cellulolytic enzyme genes cloned, CenA degraded carboxymethylcellulose and phosphoric acid swollen cellulose (PASC) efficiently. The extracellular CenA catalyzed the hydrolysis of barley β-glucan and PASC to liberate soluble cello-oligosaccharides, indicating that CenA is the most suitable enzyme for cellulose degradation among those cellulolytic enzymes expressed in Z. palmae. Furthermore, the cenA gene and β-glucosidase gene (bgl) from Ruminococcus albus were co-expressed in Z. palmae. Of the total endoglucanase and β-glucosidase activities, 57.1 and 18.1% were localized in the culture medium of the strain. The genetically engineered strain completely saccharified and fermented 20 g/l barley β-glucan to ethanol within 84 h, producing 79.5% of the theoretical yield. Thus, the production and secretion of CenA and BGL enabled Z. palmae to efficiently ferment a water-soluble cellulosic polysaccharide to ethanol.

In the thesis titled “Cloning and expression of Cellulase genes from Trichoderma reesei into Zymomonas mobilis for cellulosic ethanol production” Venkathesh S., the author reports the incorporation of cellulase genes from Trichoderma reesei into Zymomonas mobilis for the production of ethanol from cellulosic sources.

In light of the above, it is clear that there is an unmet need for a process to generate an alcohol or alcohol precursors from a lignocellulosic biomass material that is not reliant on enzymatic cocktails due to their cost. In that respect, the use of an organism that contains cellulolytic as well as fermentation capabilities is much more highly attractive as it will lower the costs of bioethanol production. Furthermore, the addition of the exogenous cellulolytic genes within this invention are genomically incorporated into the organism rather than the utilization of a plasmid based approach; which increases vastly the conferred ability without the need to continuously confirm the presence of said modifications. Additionally, the use of a low-lignin, low-hemicellulose cellulose source is extremely advantageous as it minimizes lignin-derived toxicity issues and maximizes the conversion of cellulose to alcohol and alcohol precursors.

### SUMMARY OF THE INVENTION

Preferred embodiments of the present invention are related to methods for the production of an alcohol or alcohol precursor through the conversion of cellulose-based materials by genetically modified ethanologenic organisms.

According to a preferred embodiment of the present invention, there is provided a method for the growth of said genetically modified ethanologenic organisms in conditions in which said organisms can exponentially grow to create a propagation culture. In another aspect of the present invention, said conditions optionally may lead the organism to produce alcohol and/or alcohol precursor molecules.

According to a preferred embodiment of the present invention, there is provided a method for the degradation of cellulose to a degraded cellulose mixture using said genetically modified ethanologenic organisms through the introduction of cellulolytic genes isolated from prokaryotic and/or eukaryotic organisms.

According to a preferred embodiment of the present invention, there is provided a method for the fermentation conditions using a cellulose or degraded cellulose mixture that allow said genetically modified organisms to produce alcohol and/or alcohol precursor molecules.

According to a preferred embodiment of the present invention, there is provided a method of growing an ethanologenic organism, wherein the method comprises the steps of:
exposing said live ethanologenic organism to a culture media with a pH of between 2 and 9 comprising a carbon source and a nitrogen source, thereby creating an incubation mixture; and
incubating said live ethanologenic organism in said incubation mixture under aerobic and/or anaerobic conditions at a temperature ranging from 0° C. to 60° C. for a period of time sufficient to allow for the exponential growth of said live ethanologenic organism.
   and wherein said live ethanologenic organism comprises at least one of the following polynucleotide sequences in its genome:
i. an endoglucanase (cen-like) polynucleotide sequence selected from the group consisting of an endoglucanase A-like (cenA-like) polynucleotide sequence, an endoglucanase B-like (cenB-like) polynucleotide sequence and an endoglucanase C-like (cenC-like) polynucleotide sequence;
ii. an exoglucanase (cex-like) polynucleotide sequence; and
iii. a β-glucosidase 1 (bgl1) polynucleotide sequence.
   and wherein said live ethanologenic organism belongs to a genus is selected from the group consisting of: Aspergillus, Mucor, Zymomonas, Escherichia, Clostridia, Bacillus, and Pseudomonas.

According to a preferred embodiment of the present invention, the carbon source is selected from the group consisting of monosaccharides; di-saccharides; polysaccharides; and combinations thereof. Non-limiting examples of said sugar source are long-chain saccharides, short chain saccharides, monosaccharides, sugar alcohols, organic acids and their corresponding salts and/or combinations thereof. According to a more preferred embodiment of the present invention, the carbon source is selected from the group comprising of: glucose, mannose, maltose, lactose, galactose, fructose, sucrose, molasses, ribose, arabinose, xylose, gluconic acid and gluconate salts, arabitol, mannitol, sorbitol, maltitol, xylitol, erythritol, lactitol, acetic acid, acetate salts, starch and starch hydrolysates, corn steep liquor (CSL), whey or whey permeate, bagasse hydrolysate, glycerol, fumaric acid and its salts, lactic acid and its salts, ethanol, methanol, and/or combinations thereof.

According to a preferred embodiment of the present invention, the nitrogen source is selected from the group consisting of: ammonia; inorganic and organic ammonium salts; nitrite and nitrate salts; urea; amines and amino acids; peptides; extracts from animal and plant-based industries (i.e., meat and/or yeast extract, soybean meal, corn meal, corn flour, soybean flour, gelatin, collagen, etc.); and combinations thereof.

According to a preferred embodiment of the present invention, the pH of the culture media ranges from 2 to 9. Preferably, the pH of the culture media ranges between 4 and 7. More preferably, the pH of the culture media ranges between 4.5 and 6.

According to a preferred embodiment of the present invention, the temperature of the incubation mixture ranges from 0 to 60° C. Preferably, the temperature of the incubation mixture ranges from 15 to 40° C. More preferably, the temperature of the incubation mixture ranges from 20 to 35° C.

According to another aspect of the present invention, there is provided a method of producing an alcohol or alcohol precursor from a cellulosic material and a genetically modified live ethanologenic organism, wherein the method comprises the steps of:
exposing said genetically modified live ethanologenic organism to a culture media with a pH of between 2 and 9, thereby creating an incubation mixture;
exposing said incubation mixture to a source of cellulose;
incubating said genetically modified live ethanologenic organism in said incubation mixture with said source of cellulose under aerobic and/or anaerobic conditions at a temperature ranging from 0° C. to 60° C. for a first period of time sufficient for the degradation of cellulose, thereby creating a degraded cellulose mixture;
optionally, incubating said genetically modified live ethanologenic organism in said degraded cellulose mixture under aerobic and/or anaerobic conditions at a temperature ranging from 0° C. to 60° C. for a second period of time sufficient for the production of said alcohol or alcohol precursor; and
optionally, recovering said alcohol or alcohol precursor from the cells and/or spent culture media;
   wherein said genetically modified live ethanologenic organism comprises at least one of the following polynucleotide sequences in its genome:
i. at least one endoglucanase (cen-like) polynucleotide sequence selected from the group consisting of an endoglucanase A-like (cenA-like) polynucleotide sequence, an endoglucanase B-like (cenB-like) polynucleotide sequence and an endoglucanase C-like (cenC-like) polynucleotide sequence;
ii. an exoglucanase (cex-like) polynucleotide sequence; and
iii. a β-glucosidase 1 (bgl1) polynucleotide sequence.
   and wherein said source of cellulose has a lignin content of at most 1 wt. % and a hemicellulose content of at most 15 wt. %.

According to a preferred embodiment of the present invention said live ethanologenic organism can be re-exposed to an unused or a spent media for the continuous production of an alcohol or alcohol precursor.

According to another aspect of the present invention, there is provided a method of producing an alcohol or alcohol precursor from a lignocellulosic material and a genetically modified live ethanologenic organism, wherein the method comprises the steps of:
providing a lignocellulosic biomass;
exposing said lignocellulosic biomass to a modified Caro's acid composition for a period of time necessary to remove more than 98.5% of the lignin present in said lignocellulosic biomass and thus obtaining a source of cellulose and a liquid stream;
separating said solid stream comprising said source of cellulose from said liquid stream comprising said lignin;
exposing said source of cellulose to said genetically modified live ethanologenic organism in a culture media with a pH of between 2 and 9, thereby creating an incubation mixture;
incubating said genetically modified live ethanologenic organism in said incubation mixture with said source of cellulose under aerobic and/or anaerobic conditions at a temperature ranging from 0° C. to 60° C. for a first period of time sufficient for the degradation of cellulose, thereby creating a degraded cellulose mixture;
optionally, incubating said genetically modified live ethanologenic organism in said degraded cellulose mixture under aerobic and/or anaerobic conditions at a temperature ranging from 0° C. to 60° C. for a second period of time sufficient for the production of said alcohol or alcohol precursor; and
optionally, recovering said alcohol or alcohol precursor from the cells and/or spent culture media;
   and wherein said genetically modified live ethanologenic organism comprises at least one of the following polynucleotide sequences in its genome:
i. at least one endoglucanase (cen-like) polynucleotide sequence selected from the group consisting of an endoglucanase A-like (cenA-like) polynucleotide sequence, an endoglucanase B-like (cenB-like) polynucleotide sequence and an endoglucanase C-like (cenC-like) polynucleotide sequence;
ii. an exoglucanase (cex-like) polynucleotide sequence; and
iii. a β-glucosidase 1 (bgl1) polynucleotide sequence;
   and wherein said live ethanologenic organism belongs to a genus is selected from the group consisting of: Aspergillus, Mucor, Zymomonas, Escherichia, Clostridia, Bacillus, and Pseudomonas.

In some embodiments of the present invention, the process of exposing said lignocellulosic biomass to a modified Caro's acid composition to delignify the former can be carried out for a varying duration of time depending on the particle size of the biomass and the type of biomass being fed into the process. In some cases, the process can last from 2 to 20 hours depending on that characteristic. The process is preferably run at temperatures below 50° C., more preferably at temperatures below 40° C.

According to a preferred embodiment of the present invention, an alcohol precursor refers to any molecule that is obtained as an intermediate, by-product or co-product of the conversion of a cellulosic material to an alcohol. It is known to the person skilled in the art that said alcohol precursor can be a polysaccharide, oligosaccharide, di-saccharide, monosaccharide, organic acid and their corresponding salts, aldehyde, ketone, etc. Preferably, said alcohol precursor is selected from the group consisting of: cellobiose, glucose, and ethanol. Preferably, said alcohol or alcohol precursor is ethanol.

According to a preferred embodiment of the present invention, the pH of the culture media ranges from 2 to 9. Preferably, the pH of the culture media ranges between 4 and 7. More preferably, the pH of the culture media ranges between 4.5 and 6.

According to a preferred embodiment of the present invention, said source of cellulose has been obtained from the delignification of biomass using a modified Caro's acid composition. According to another embodiment of the present invention, said source of cellulose comprises less than 15% wt. of hemicellulose. Preferably, the solid stream comprises less than 10% wt. of hemicellulose. More preferably, the solid stream comprises less than 5% wt. of hemicellulose. It is widely accepted that a kappa number is a reliable indication of lignin content in a pulp or cellulosic material. The higher the kappa number, the higher the lignin content is. According to a preferred embodiment of the present invention, said source of cellulose has a Kappa number of 0-7, preferably less than 5 and even more preferably, less than 2.

According to a preferred embodiment of the present invention, the temperature of the incubation mixture ranges from 0 to 60° C. Preferably, the temperature of the incubation mixture ranges from 15 to 50° C. More preferably, the temperature of the incubation mixture ranges from 20 to 40° C.

According to a preferred method of the present invention, the ethanologenic organism is selected from the genus of Aspergillus, Mucor, Zymomonas, Escherichia, Clostridia, Bacillus, and Pseudomonas, however this list by no means is meant to limit the scope of the invention. Preferably, the ethanologenic organism is selected from the genus Zymomonas. Even more preferably, the ethanologenic organism is the bacteria Zymomonas mobilis.

According to a preferred embodiment of the present invention, said ethanologenic organism preferentially utilizes a source of cellulose comprising a low lignin content. By exposing a lignocellulosic biomass to a modified Caro's acid as per a delignification method described herein, most of the lignin, which is non-degradable by said organism, is removed therefore allowing for a more optimal and efficient degradation of the remaining cellulosic portion due to the bioavailability of the carbohydrate-based portion. When lignin is tightly bonded within the cellulosic fibers, it renders its degradation difficult and inefficient; thus, removing the lignin according to a method described herein is important for the biodegradation into value-added products. According to another aspect of the present invention, said ethanologenic organism preferentially utilizes a source of cellulose containing a low hemicellulose content due to its type of metabolism. It is known to those skilled in the art that pentoses, obtained from the degradation of bemicellulose, require of a specific metabolic pathway not present in many microorganisms for its utilization. By utilizing a low-lignin and low-hemicellulose cellulosic portion, the method described herein allows for the full conversion of said cellulose to value added products by a vast majority of ethanologenic organisms.

It is known to the person skilled in the art that a plethora of different techniques can be utilized for the extraction and purification of the desired alcohol or alcohol precursor from the cells or the mixture in which the cells are. These methods include, but are not limited to centrifugation, filtration, dialysis, crystallization, ion exchange, electrodialysis, solvent extraction, evaporation, liquid-liquid extraction, distillation, and nanofiltration.

### BRIEF DESCRIPTION OF THE ACCOMPANYING FIGURE

The invention may be more completely understood in consideration of the following description of various embodiments of the invention in connection with the accompanying figure, in which:

FIG. 1 is a schematic representation of the differences in the linkages between individual sugar units in starch and in cellulose.

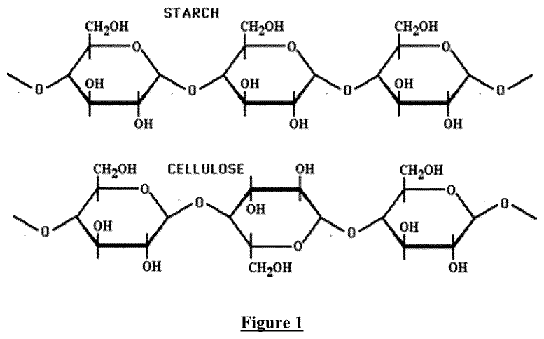

### DETAILED DESCRIPTION OF THE INVENTION

According to another aspect of the present invention, there is provided a method of producing an alcohol or alcohol precursor from a cellulosic material and a genetically modified live ethanologenic organism, wherein the method comprises the steps of:
exposing said genetically modified live ethanologenic organism to a culture media with a pH of between 2 and 9, thereby creating an incubation mixture;
exposing said incubation mixture to a source of cellulose;
incubating said genetically modified live ethanologenic organism in said incubation mixture with said source of cellulose under aerobic and/or anaerobic conditions at a temperature ranging from 0° C. to 60° C. for a first period of time sufficient for the degradation of cellulose, thereby creating a degraded cellulose mixture;
optionally, incubating said genetically modified live ethanologenic organism in said degraded cellulose mixture under aerobic and/or anaerobic conditions at a temperature ranging from 0° C. to 60° C. for a second period of time sufficient for the production of said alcohol or alcohol precursor; and
optionally, recovering said alcohol or alcohol precursor from the cells and/or spent culture media;
   wherein said genetically modified live ethanologenic organism comprises at least one of the following polynucleotide sequences in its genome:
iv. at least one endoglucanase (cen-like) polynucleotide sequence selected from the group consisting of an endoglucanase A-like (cenA-like) polynucleotide sequence, an endoglucanase B-like (cenB-like) polynucleotide sequence and an endoglucanase C-like (cenC-like) polynucleotide sequence;
v. an exoglucanase (cex-like) polynucleotide sequence; and
vi. a β-glucosidase 1 (bgl1) polynucleotide sequence;

and wherein said source of cellulose has a lignin content of at most 1 wt. % and a hemicellulose content of at most 15 wt. %.

According to another aspect of the present invention, there is provided a method of producing an alcohol or alcohol precursor from a lignocellulosic material and a genetically modified live ethanologenic organism, wherein the method comprises the steps of:
providing a lignocellulosic biomass
exposing said lignocellulosic biomass to a modified Caro's acid composition for a period of time necessary to remove more than 98.5% of the lignin present in said lignocellulosic biomass and thus obtaining a source of cellulose and a liquid stream,
separating said source of cellulose from said liquid stream;
exposing said source of cellulose to said genetically modified live ethanologenic organism in a culture media with a pH of between 2 and 9, thereby creating an incubation mixture;
incubating said genetically modified live ethanologenic organism in said incubation mixture with said source of cellulose under aerobic and/or anaerobic conditions at a temperature ranging from 0° C. to 60° C. for a first period of time sufficient for the degradation of cellulose, thereby creating a degraded cellulose mixture;
optionally, incubating said genetically modified live ethanologenic organism in said degraded cellulose mixture under aerobic and/or anaerobic conditions at a temperature ranging from 0° C. to 60° C. for a second period of time sufficient for the production of said alcohol or alcohol precursor; and
optionally, recovering said alcohol or alcohol precursor from the cells and/or spent culture media;
   and wherein said genetically modified live ethanologenic organism comprises at least one of the following polynucleotide sequences:
iv. at least one endoglucanase (cen-like) polynucleotide sequence selected from the group consisting of an endoglucanase A-like (cenA-like) polynucleotide sequence, an endoglucanase B-like (cenB-like) polynucleotide sequence and an endoglucanase C-like (cenC-like) polynucleotide sequence;
v. an exoglucanase (cex-like) polynucleotide sequence; and
vi. a β-glucosidase 1 (bgl1) polynucleotide sequence;

and wherein said live ethanologenic organism belongs to a genus is selected from the group consisting of: Aspergillus, Mucor, Zymomonas, Escherichia, Clostridia, Bacillus, and Pseudomonas.

According to a preferred embodiment of the present invention, said source of cellulose may be neutralized prior to exposing it to a said genetically modified live ethanologenic organism in a culture media. Preferably, said source of cellulose is neutralized to a pH between 2 and 9. More preferably, said source of cellulose is neutralized to a pH between 5 and 7.

In some embodiments of the present invention, said lignocellulosic biomass may be mechanically treated to reduce particle size prior to contacting it to a modified Caro's acid.

According to preferred embodiment of the present invention, the modified Caro's acid (as disclosed in Canadian patent application 3,128,678) comprises: sulfuric acid; a heterocyclic compound; and wherein sulfuric acid and said a heterocyclic compound; are present in a molar ratio of no less than 1:1. Preferably, the sulfuric acid and said heterocyclic compound are present in a molar ratio ranging from 28:1 to 2:1. More preferably, the sulfuric acid and heterocyclic compound are present in a molar ratio ranging from 24:1 to 3:1. Preferably, the sulfuric acid and heterocyclic compound are present in a molar ratio ranging from 20:1 to 4:1. More preferably, the sulfuric acid and heterocyclic compound are present in a molar ratio ranging from 16:1 to 5:1. Preferably, the sulfuric acid and heterocyclic compound are present in a molar ratio ranging from 12:1 to 6:1. Also preferably, said heterocyclic compound has a molecular weight below 300 g/mol. Also preferably, said heterocyclic compound has a molecular weight below 150 g/mol. More preferably, said heterocyclic compound is a secondary amine. According to a preferred embodiment of the present invention, said heterocyclic compound is selected from the group consisting of: imidazole; triazole; and N-methylimidazole.

According to preferred embodiment of the present invention, the modified Caro's acid (as disclosed in Canadian patent application 3,128,677) comprises: sulfuric acid; a modifying agent comprising a compound containing an amine group; and wherein sulfuric acid and said compound containing an amine group; are present in a molar ratio of no less than 1:1. Preferably, the sulfuric acid and said compound containing an amine group are present in a molar ratio ranging from 28:1 to 2:1. More preferably, the sulfuric acid and compound containing an amine group are present in a molar ratio ranging from 24:1 to 3:1. Preferably, the sulfuric acid and compound containing an amine group are present in a molar ratio ranging from 20:1 to 4:1. More preferably, the sulfuric acid and compound containing an amine group are present in a molar ratio ranging from 16:1 to 5:1. Preferably, the sulfuric acid and compound containing an amine group are present in a molar ratio ranging from 12:1 to 6:1. According to a preferred embodiment of the present invention, the modifying agent is selected in the group consisting of: TEOA; MEOA; pyrrolidine; DEOA; ethylenediamine; diethylamine; triethylamine; morpholine; MEA-triazine; and combinations thereof. According to a more preferred embodiment of the present invention, the modifying agent is TEOA; MEOA; pyrrolidine; DEOA; ethylenediamine; triethylamine.

According to preferred embodiment of the present invention, the modified Caro's acid (as disclosed in Canadian patent application 3,128,676) comprises: sulfuric acid; a modifying agent comprising an alkanesulfonic acid; and wherein sulfuric acid and said alkanesulfonic acid are present in a molar ratio of no less than 1:1. Preferably, said alkanesulfonic acid is selected from the group consisting of: alkanesulfonic acids where the alkyl groups range from C₁-C₆ and are linear or branched; and combinations thereof. Preferably, said alkanesulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof. More preferably, said alkanesulfonic acid is methanesulfonic acid. Also preferably, said alkanesulfonic acid has a molecular weight below 300 g/mol. Also preferably, said alkanesulfonic acid has a molecular weight below 150 g/mol. Preferably, the sulfuric acid and said alkanesulfonic acid and are present in a molar ratio ranging from 28:1 to 2:1. More preferably, the sulfuric acid and alkanesulfonic acid are present in a molar ratio ranging from 24:1 to 3:1. Preferably, the sulfuric acid and alkanesulfonic acid are present in a molar ratio ranging from 20:1 to 4:1. More preferably, the sulfuric acid and alkanesulfonic acid are present in a molar ratio ranging from 16:1 to 5:1. According to a preferred embodiment of the present invention, the sulfuric acid and alkanesulfonic acid are present in a molar ratio ranging from 12:1 to 6:1.

According to preferred embodiment of the present invention, the modified Caro's acid (as disclosed in Canadian patent application 3,128,675) comprises: sulfuric acid; a substituted aromatic compound; and wherein sulfuric acid and said substituted aromatic compound; are present in a molar ratio of no less than 1:1. Preferably, the substituted aromatic compound comprises at least two substituents. More preferably, at least one substituent is an amine group and at least one of the other substituent is a sulfonic acid moiety. According to a preferred embodiment, the substituted aromatic compound comprises three or more substituent. According to a preferred embodiment of the present invention, the substituted aromatic compound comprises at least a sulfonic acid moiety. According to another preferred embodiment of the present invention, the substituted aromatic compound comprises an aromatic compound having a sulfonamide substituent, where the compound can be selected from the group consisting of: benzenesulfonamides; toluenesulfonamides; substituted benzenesulfonamides; and substituted toluenesulfonamides. Preferably, the sulfuric acid and said substituted aromatic compound and are present in a molar ratio ranging from 28:1 to 2:1. More preferably, the sulfuric acid and substituted aromatic compound are present in a molar ratio ranging from 24:1 to 3:1. Preferably, the sulfuric acid and substituted aromatic compound are present in a molar ratio ranging from 20:1 to 4:1. More preferably, the sulfuric acid and substituted aromatic compound are present in a molar ratio ranging from 16:1 to 5:1. Preferably, the sulfuric acid and substituted aromatic compound are present in a molar ratio ranging from 12:1 to 6:1.

According to preferred embodiment of the present invention, the modified Caro's acid (as disclosed in Canadian patent application 3,128,674) comprises: sulfuric acid; a modifying agent comprising an arylsulfonic acid; and optionally, a compound containing an amine group; wherein sulfuric acid and said a arylsulfonic acid; are present in a molar ratio of no less than 1:1. Preferably, the compound containing an amine group is selected from the group consisting of: imidazole; N-methylimidazole; triazole; monoethanolamine (MEOA); diethanolamine (DEOA); triethanolamine (TEOA); pyrrolidine and combinations thereof. According to a preferred embodiment of the present invention, sulfuric acid and the peroxide are present in a molar ratio of approximately 1:1. Preferably, the sulfuric acid and said arylsulfonic acid and are present in a molar ratio ranging from 28:1 to 2:1. More preferably, the sulfuric acid and arylsulfonic acid are present in a molar ratio ranging from 24:1 to 3:1. Preferably, the sulfuric acid and arylsulfonic acid are present in a molar ratio ranging from 20:1 to 4:1. More preferably, the sulfuric acid and arylsulfonic acid are present in a molar ratio ranging from 16:1 to 5:1. According to a preferred embodiment of the present invention, the sulfuric acid and arylsulfonic acid are present in a molar ratio ranging from 12:1 to 6:1. Also preferably, said arylsulfonic acid has a molecular weight below 300 g/mol. Also preferably, said arylsulfonic acid has a molecular weight below 150 g/mol. Even more preferably, said arylsulfonic acid is selected from the group consisting of: orthanilic acid; metanilic acid; sulfanilic acid; toluenesulfonic acid; benzenesulfonic acid; and combinations thereof.

According to preferred embodiment of the present invention, the modified Caro's acid (as disclosed in Canadian patent application 3,128,673) comprises: sulfuric acid; a heterocyclic compound; an alkanesulfonic acid; and wherein sulfuric acid and said a heterocyclic compound; are present in a molar ratio of no less than 1:1. Preferably, said aqueous acidic composition comprising: sulfuric acid; a heterocyclic compound; an arylsulfonic acid; and wherein sulfuric acid and said a heterocyclic compound; are present in a molar ratio of no less than 1:1. Preferably, the arylsulfonic acid is toluenesulfonic acid.

Preferably, the sulfuric acid, the heterocyclic compound and the alkanesulfonic acid are present in a molar ratio ranging from 28:1:1 to 2:1:1. More preferably, the sulfuric acid the heterocyclic compound and the alkanesulfonic acid are present in a molar ratio ranging from 24:1:1 to 3:1:1. Preferably, the sulfuric acid, the heterocyclic compound and the alkanesulfonic acid are present in a molar ratio ranging from 20:1:1 to 4:1:1. More preferably, the sulfuric acid, the heterocyclic compound and the alkanesulfonic acid are present in a molar ratio ranging from 16:1:1 to 5:1:1. According to a preferred embodiment of the present invention, the sulfuric acid and heterocyclic compound are present in a molar ratio ranging from 12:1:1 to 6:1:1. Also preferably, said heterocyclic compound has a molecular weight below 300 g/mol. Also preferably, said heterocyclic compound has a molecular weight below 150 g/mol. Even more preferably, said heterocyclic compound is selected from the group consisting of: imidazole; triazole; n-methylimidazole; and combinations thereof. Preferably, the alkanesulfonic acid is selected from the group consisting of:
alkylsulfonic acids where the alkyl groups range from C₁-C₆ and are linear or branched; and combinations thereof. Preferably, said alkylsulfonic acid is selected from the group consisting of:
methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof. More preferably, said alkylsulfonic acid is methanesulfonic acid.

According to preferred embodiment of the present invention, the modified Caro's acid (as disclosed in Canadian patent application 3,128,672) comprises: sulfuric acid; a carbonyl-containing nitrogenous base compound; and wherein sulfuric acid and said a carbonyl-containing nitrogenous base compound; are present in a molar ratio of no less than 1:1. According to a preferred embodiment of the present invention, the carbonyl-containing nitrogenous base compound is selected from the group consisting of: caffeine; lysine; creatine; glutamine; creatinine; 4-aminobenzoic acid; glycine; NMP (N-methyl-2-pyrrolidinone); histidine; DMA (N,N-dimethylacetamide); arginine; 2,3-pyridinedicarboxylic acid; hydantoin; and combinations thereof. Preferably, the sulfuric acid and said carbonyl-containing nitrogenous base compound and are present in a molar ratio ranging from 28:1 to 2:1. More preferably, the sulfuric acid and carbonyl-containing nitrogenous base compound are present in a molar ratio ranging from 24:1 to 3:1. Preferably, the sulfuric acid and carbonyl-containing nitrogenous base compound are present in a molar ratio ranging from 20:1 to 4:1. More preferably, the sulfuric acid and carbonyl-containing nitrogenous base compound are present in a molar ratio ranging from 16:1 to 5:1. According to a preferred embodiment of the present invention, the sulfuric acid and carbonyl-containing nitrogenous base compound are present in a molar ratio ranging from 12:1 to 6:1.

According to a preferred embodiment of the present invention, said lignocellulosic biomass comprising lignin, hemicellulose and cellulose is exposed to a modified Caro's acid composition having a pH of less than 1, said modified Caro's acid composition selected from the group consisting of: composition A; composition B; composition C; composition D; composition E; composition F; composition G; composition H; composition I; and composition J; wherein said composition A comprises:
sulfuric acid;
a compound comprising an amine moiety and a sulfonic acid moiety; and
a peroxide; and wherein sulfuric acid, said compound comprising an amine moiety and a sulfonic acid moiety and said peroxide are present in a molar ratio of no less than 1:1:1;
   wherein said composition B comprises:
sulfuric acid;
a compound comprising an amine moiety;
a compound comprising a sulfonic acid moiety; and
a peroxide; wherein sulfuric acid and said a compound comprising an amine moiety and said compound comprising a sulfonic acid moiety are present in a molar ratio of no less than 1:1:1;
   wherein said composition C comprises:
an alkylsulfonic acid; and
a peroxide; wherein said alkylsulfonic acid and said peroxide are present in a molar ratio of no less than 1:1;
   wherein said composition D comprises:
sulfuric acid;
a heterocyclic compound; and
a peroxide; and wherein sulfuric acid and said a heterocyclic compound; are present in a molar ratio of no less than 1:1;
   wherein said composition E comprises:
sulfuric acid;
a modifying agent comprising a compound containing an amine group; and
a peroxide; and wherein sulfuric acid and said compound containing an amine group; are present in a molar ratio of no less than 1:1;
   wherein said composition F comprises:
sulfuric acid;
a modifying agent comprising an alkanesulfonic acid and
a peroxide; and wherein sulfuric acid and said alkanesulfonic acid are present in a molar ratio of no less than 1:1;
   wherein said composition G comprises:
sulfuric acid;
a substituted aromatic compound; and
a peroxide; and wherein sulfuric acid and said substituted aromatic compound; are present in a molar ratio of no less than 1:1;
   wherein said composition H comprises:
sulfuric acid;
a modifying agent comprising an arylsulfonic acid;
a peroxide; and
optionally, a compound containing an amine group; wherein sulfuric acid and said arylsulfonic acid are present in a molar ratio of no less than 1:1;
   wherein said composition I comprises:
sulfuric acid;
a heterocyclic compound;
an alkanesulfonic acid; and
a peroxide; and wherein sulfuric acid and said a heterocyclic compound; are present in a molar ratio of no less than 1:1;
   wherein said composition J comprises:
sulfuric acid;
a carbonyl-containing nitrogenous base compound; and
a peroxide; and wherein sulfuric acid and said a carbonyl-containing nitrogenous base compound; are present in a molar ratio of no less than 1:1.

According to a preferred embodiment of the present invention, the lignocellulosic biomass mixture comprising hemicellulose, lignin, and cellulose is exposed to a modified Caro's acid composition at a temperature and for a period of time sufficient to a delignification reaction to occur and remove over 98.5% wt. of said lignin and over 70% wt. of the hemicellulose in a liquid stream preferably leaving in solid form most of the cellulose from said biomass.

Preferably, said compound comprising an amine moiety and a sulfonic acid moiety is selected from the group consisting of taurine; taurine derivatives; and taurine-related compounds.

Preferably, said taurine derivative or taurine-related compound is selected from the group consisting of: taurolidine; taurocholic acid; tauroselcholic acid; tauromustine; 5-taurinomethyluridine and 5-taurinomethyl-2-thiouridine; homotaurine (tramiprosate); acamprosate; and taurates as well as aminoalkylsulfonic acids, where the alkyl is selected from the group consisting of C₁-C₅ linear alkyl and C₁-C₅ branched alkyl. Preferably, said linear alkylaminosulfonic acid is selected from the group consisting of: methyl; ethyl (taurine); propyl; and butyl. Preferably, said branched aminoalkylsulfonic acid is selected from the group consisting of: isopropyl; isobutyl; and isopentyl.

According to a preferred embodiment of the present invention, said compound comprising an amine moiety and a sulfonic acid moiety is taurine.

According to a preferred embodiment of the present invention, said sulfuric acid and a compound comprising an amine moiety and a sulfonic acid moiety are present in a molar ratio of no less than 3:1.

According to a preferred embodiment of the present invention, said compound comprising an amine moiety is an alkanolamine is selected from the group consisting of: monoethanolamine; diethanolamine; triethanolamine; and combinations thereof.

Preferably, said compound comprising a sulfonic acid moiety is selected from the group consisting of: alkylsulfonic acids; arylsulfonic acids; and combinations thereof. Preferably, said alkylsulfonic acid is selected from the group consisting of: alkylsulfonic acids where the alkyl groups range from C₁-C₆ and are linear or branched; and combinations thereof. More preferably, said alkylsulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof. According to a preferred embodiment of the present invention, said arylsulfonic acid is selected from the group consisting of: toluenesulfonic acid; benzesulfonic acid; and combinations thereof.

According to a preferred embodiment of the present invention, the temperature of the lignocellulosic biomass mixture comprising hemicellulose, lignin and cellulose is kept below 55° C. for the duration of the delignification reaction. Preferably, the temperature of the lignocellulosic biomass mixture comprising hemicellulose, lignin and cellulose is kept below 50° C. for the duration of the delignification reaction. According to another preferred embodiment of the present invention, the temperature of the lignocellulosic biomass mixture comprising hemicellulose, lignin and cellulose is kept below 45° C. for the duration of the delignification reaction. According to a preferred embodiment of the present invention, the temperature of the lignocellulosic biomass mixture comprising hemicellulose, lignin and cellulose is kept below 40° C. for the duration of the delignification reaction.

According to a preferred embodiment of the present invention, the temperature of the remaining biomass mixture is controlled throughout the delignification reaction to subsequent additions of a solvent (water) to progressively lower the slope of temperature increase per minute from less than 1° C. per minute to less than 0.5° C. per minute.

According to another preferred embodiment of the present invention, the temperature of the lignocellulosic biomass mixture comprising hemicellulose, lignin and cellulose is controlled by an addition of a solvent (water) to reduce the slope of temperature increase per minute of the reaction mass to less than 1° C. per minute.

According to yet another preferred embodiment of the present invention, the temperature of the lignocellulosic biomass mixture comprising hemicellulose, lignin and cellulose is controlled by a second addition of a solvent (water) to reduce the slope of temperature increase per minute of the reaction mass to less than 0.7° C. per minute.

Preferably, the temperature of the lignocellulosic biomass mixture comprising hemicellulose, lignin and cellulose is controlled by a third addition of a solvent (water) to reduce the slope of temperature increase per minute of the reaction mass to less than 0.3° C. per minute.

Preferably, the temperature of the lignocellulosic biomass mixture comprising hemicellulose, lignin and cellulose is controlled by a fourth addition of a solvent (water) to reduce the slope of temperature increase per minute of the reaction mass to less than 0.1° C. per minute.

According to a preferred embodiment of the present invention, there is provided a process to delignify biomass using an aqueous acidic composition comprising:
sulfuric acid;
a heterocyclic compound; and
a peroxide.

According to another preferred embodiment of the present invention, there is provided a process to delignify biomass using an aqueous acidic composition comprising:
sulfuric acid;
a heterocyclic compound; and
   wherein sulfuric acid and said a heterocyclic compound; are present in a molar ratio of no less than 1:1.

Preferably, the sulfuric acid and said heterocyclic compound are present in a molar ratio ranging from 28:1 to 2:1. More preferably, the sulfuric acid and heterocyclic compound are present in a molar ratio ranging from 24:1 to 3:1. Preferably, the sulfuric acid and heterocyclic compound are present in a molar ratio ranging from 20:1 to 4:1. More preferably, the sulfuric acid and heterocyclic compound are present in a molar ratio ranging from 16:1 to 5:1. According to a preferred embodiment of the present invention, the sulfuric acid and heterocyclic compound are present in a molar ratio ranging from 12:1 to 6:1.

Also preferably, said heterocyclic compound has a molecular weight below 300 g/mol. Also preferably, said heterocyclic compound has a molecular weight below 150 g/mol. More preferably, said heterocyclic compound is a secondary amine. According to a preferred embodiment of the present invention, said heterocyclic compound is selected from the group consisting of: imidazole; triazole; and N-methylimidazole.

According to an aspect of the present invention, there is provided a process to delignify biomass, such as wood using an aqueous acidic composition comprising:
sulfuric acid;
a heterocyclic compound; and
a peroxide;
   wherein the sulfuric acid and the heterocyclic compound are present in a mole ratio ranging from 2:1 to 28:1.

Preferably, said sulfuric acid, said compound comprising an amine moiety and a sulfonic acid moiety and said peroxide are present in a molar ratio of no less than 1:1:1. Also preferably, said sulfuric acid, said compound comprising an amine moiety and a sulfonic acid moiety and said peroxide are present in a molar ratio of no more than 15:1:1.

According to a preferred embodiment of the present invention, said sulfuric acid and said compound comprising an amine moiety and a sulfonic acid moiety are present in a molar ratio of no less than 3:1.

According to a preferred embodiment of the present invention, said compound comprising an amine moiety and a sulfonic acid moiety is selected from the group consisting of: taurine; taurine derivatives; and taurine-related compounds.

According to a preferred embodiment of the present invention, said taurine derivative or taurine-related compound is selected from the group consisting of: taurolidine; taurocholic acid; tauroselcholic acid; tauromustine; 5-taurinomethyluridine and 5-taurinomethyl-2-thiouridine; homotaurine (tramiprosate); acamprosate; and taurates; as well as aminoalkylsulfonic acids, where the alkyl is selected from the group consisting of C₁-C₅ linear alkyl and C₃-C₅ branched alkyl. Preferably, said linear alkylaminosulfonic acid is selected from the group consisting of: methyl; ethyl (taurine); propyl; and butyl.

Preferably, said branched aminoalkylsulfonic acid is selected from the group consisting of: isopropyl; isobutyl; and isopentyl.

According to a preferred embodiment of the present invention, said compound comprising an amine moiety and a sulfonic acid moiety is taurine.

According to a preferred embodiment of the present invention, said sulfuric acid and a compound comprising an amine moiety and a sulfonic acid moiety are present in a molar ratio of no less than 3:1.

According to a preferred embodiment of the present invention, said compound comprising an amine moiety is an alkanolamine is selected from the group consisting of: monoethanolamine; diethanolamine; triethanolamine; and combinations thereof.

According to a preferred embodiment of the present invention, said compound comprising a sulfonic acid moiety is selected from the group consisting of: alkylsulfonic acids and combinations thereof.

According to a preferred embodiment of the present invention, said alkylsulfonic acid is selected from the group consisting of: alkylsulfonic acids where the alkyl groups range from C₁-C₆ and are linear or branched; and combinations thereof.

According to a preferred embodiment of the present invention, said alkylsulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof.

According to a preferred embodiment of the present invention, said alkylsulfonic acid; and said peroxide is present in a molar ratio of no less than 1:1.

According to a preferred embodiment of the present invention, said compound comprising a sulfonic acid moiety is methanesulfonic acid.

According to a preferred embodiment of the present invention, in Composition C, said sulfuric acid and said a compound comprising an amine moiety and said compound comprising a sulfonic acid moiety are present in a molar ratio of no less than 1:1:1.

According to a preferred embodiment of the present invention, in Composition C, said sulfuric acid, said compound comprising an amine moiety and said compound comprising a sulfonic acid moiety are present in a molar ratio ranging from 28:1:1 to 2:1:1.

In a preferred embodiment of the present invention, the endoglucanase A-like (cenA-like) polynucleotide sequence utilized is SEQ 1. However, in other embodiments of the invention, polynucleotide sequences that are homologous and/or substantially similar to SEQ 1 may also be used. In another embodiment of the present invention, polynucleotide sequences for endoglucanase A-like (cenA-like) in these embodiments will, preferably, have at least 70% sequence coverage, or more preferably greater than 80%, 90%, 95%, 98%, or most preferentially greater than 99% sequence coverage to SEQ 1, and sequence identities of at least 70%, or more preferentially greater than 80%, 90%, 95%, 97% sequence identity, and most preferentially 99% sequence identity to SEQ 1. These polynucleotide sequences may include, but by no means limited to, the following sequences: SEQ1; SEQ 21; SEQ 22; SEQ 23; SEQ 24; SEQ 25; SEQ 26; and SEQ 27.

According to a preferred embodiment of the present invention, cenA-like genes can be acquired from, but by no means limited to, the following bacterial species: Cellulomonas uda, Cellulomonas palmilytica, Cellulomonas gelida, Cellulomonas xiejunii, Neisseria sicca, Sanguibacter suaedae, Isoptericola dokdonensis, Sanguibacter antarcticus, Cryptosporangium aurantiacum, Isoptericola halotolerans, Nocardiopsis lucentensis, and Pseudoduganella lutea. These listed organisms are exemplary only and are in no way meant to limit what organisms these genes can be acquired from, nor to limit the scope of the invention.

According to a preferred embodiment of the present invention, SEQ 1, upon transcription and translation, provides an endoglucanase A-like (CENA-like) polypeptide sequence SEQ 2. However, according to other preferred embodiments of the invention, polypeptide sequences that are homologous and/or substantially similar to SEQ 2 may also be used in the present invention to produce an alcohol or alcohol precursor. Polypeptide sequences for endoglucanase A-like in these embodiments will, preferably, have at least 70% sequence coverage, or more preferentially greater than 80%, 90%, 95%, 98%, or most preferentially greater than 99% sequence coverage to SEQ 2, and a sequence identity of, preferably, at least 30% to SEQ 2, or more preferentially greater than 35%, 40%, 45%, 50%, 55%, 60%, 65%, 70% 75%, 80%, 85%, 90%, 95%, 97%, or most preferentially greater than 99% sequence identity to SEQ 2. These polypeptide sequences may include, but are not limited to, the following sequences: SEQ 2; SEQ 28; SEQ 29; SEQ 30; SEQ 31; SEQ 32; SEQ 33; SEQ 34; SEQ 35; SEQ 36; SEQ 37; SEQ 38; SEQ 39; SEQ 40;SEQ 41; SEQ 42; SEQ 43; SEQ 44; and SEQ 45.

In a preferred embodiment of the present invention, the endoglucanase B-like (cenB-like) polynucleotide sequence utilized is SEQ 3. However, in other embodiments of the invention, polynucleotide sequences that are homologous and/or substantially similar to SEQ 3 may also be used. In another embodiment of the present invention, polynucleotide sequences for endoglucanase B-like (cenB-like) in these embodiments will, preferably, have at least 70% sequence coverage, or more preferably greater than 80%, 90%, 95%, 98%, or most preferentially greater than 99% sequence coverage to SEQ 3, and sequence identities of at least 70%, or more preferentially greater than 80%, 90%, 95%, 97% sequence identity, and most preferentially 99% sequence identity to SEQ 3. These polynucleotide sequences may include, but by no means limited to, the following sequences: SEQ 3; SEQ 46; SEQ 47; SEQ 48; SEQ 49; SEQ 50; and SEQ 51.

According to a preferred embodiment of the present invention, cenB-like genes can be acquired from, but by no means limited to, the following bacterial species: Cellulomonas uda, Cellulomonas gelida, Micromonospora luteifusca, Micromonospora orduensis, Asanoa ishikariensis, Catelliglobosispora koreensis, Roseateles terrae, Thermobifida halotolerans, Catellatospora tritici, Micromonospora noduli, Anaerolineae bacterium, Reinekea marinisedimentorum, and Evansella caseinilytica. These listed organisms are exemplary only and are in no way meant to limit what organisms these genes can be acquired from, nor to limit the scope of the invention.

According to a preferred embodiment of the present invention. SEQ 3, upon transcription and translation, provides an endoglucanase B-like (CENB-like) polypeptide sequence SEQ 4. However, according to other preferred embodiments of the invention, polypeptide sequences that are homologous and/or substantially similar to SEQ 4 may also be used in the present invention to produce an alcohol or alcohol precursor. Polypeptide sequences for endoglucanase B-like in these embodiments will, preferably, have at least 70% sequence coverage, or more preferentially greater than 80%, 90%, 95%, 98%, or most preferentially greater than 99% sequence coverage to SEQ 4, and a sequence identity of, preferably, at least 30% to SEQ 4, or more preferentially greater than 35%, 40%, 45%, 50%, 55%, 60%, 65%, 70% 75%, 80%, 85%, 90%, 95%, 97%, or most preferentially greater than 99% sequence identity to SEQ 4. These polypeptide sequences may include, but are not limited to, the following sequences: SEQ 4; SEQ 52; SEQ 53; SEQ 54; SEQ 55; SEQ 56; SEQ 57; SEQ 58; SEQ 59; SEQ 60; SEQ 61; SEQ 62; SEQ 63; SEQ 64; SEQ 65; SEQ 64; SEQ 65; SEQ 66; SEQ 67; SEQ 68; SEQ 69; SEQ 70; SEQ 71; SEQ 72; SEQ 73; SEQ 74; and SEQ 75.

In a preferred embodiment of the present invention, the endoglucanase C-like (cenC-like) polynucleotide sequence utilized is SEQ 5. However, in other embodiments of the invention, polynucleotide sequences that are homologous and/or substantially similar to SEQ 5 may also be used. In another embodiment of the present invention, polynucleotide sequences for endoglucanase C-like (cenC-like) in these embodiments will, preferably, have at least 70% sequence coverage, or more preferably greater than 80%, 90%, 95%, 98%, or most preferentially greater than 99% sequence coverage to SEQ 5, and sequence identities of at least 70%, or more preferentially greater than 80%, 90%, 95%, 97% sequence identity, and most preferentially 99% sequence identity to SEQ 5. These polynucleotide sequences may include, but by no means limited to, the following sequences: SEQ5; SEQ 76; and SEQ 77.

According to a preferred embodiment of the present invention, cenC-like genes can be acquired from, but by no means is limited to, the following bacterial species: Cellulomonas uda, Cellulomonas palmilytica, Cellulomonas fulva, Zhihengliuella halotolerans, Saccharothrix ecbatanensis, Cellulomonas terrae, Ruania rhizosphaerae, Alteromonadaceae bacterium Bs31, and Catellatospora tritici. These listed organisms are exemplary only and are in no way meant to limit what organisms these genes can be acquired from, nor to limit the scope of the invention.

According to a preferred embodiment of the present invention, SEQ 5, upon transcription and translation, provides an endoglucanase C-like (CENC-like) polypeptide sequence SEQ 6. However, according to other preferred embodiments of the invention, polypeptide sequences that are homologous and/or substantially similar to SEQ 6 may also be used in the present invention to produce an alcohol or alcohol precursor. Polypeptide sequences for endoglucanase C-like in these embodiments will, preferably, have at least 70% sequence coverage, or more preferentially greater than 80%, 90%, 95%, 98%, or most preferentially greater than 99% sequence coverage to SEQ 6, and a sequence identity of, preferably, at least 30% to SEQ 6, or more preferentially greater than 35%, 40%, 45%, 50%, 55%, 60%, 65%, 70% 75%, 80%, 85%, 90%, 95%, 97%, or most preferentially greater than 99% sequence identity to SEQ 6. These polypeptide sequences may include, but are not limited to, the following sequences: SEQ 6; SEQ 78; SEQ 79; SEQ 80; SEQ 81; SEQ 82; SEQ 83; SEQ 84; SEQ 85; SEQ 86; SEQ 87; SEQ 88; SEQ 89; SEQ 90; SEQ 91; SEQ 92; and SEQ 93.

In a preferred embodiment of the present invention, the β-glucosidase 1 (bgl1) polynucleotide sequence utilized is SEQ 9 or SEQ 20. However, in other embodiments of the invention, polynucleotide sequences that are homologous and/or substantially similar to SEQ 9 or SEQ 20 may also be used. In another embodiment of the present invention, polynucleotide sequences for β-glucosidase 1 (bgl1) in these embodiments will, preferably, have at least 70% sequence coverage, or more preferably greater than 80%, 90%, 95%, 98%, or most preferentially greater than 99% sequence coverage to SEQ 9 or SEQ 20, and sequence identities of at least 70%, or more preferentially greater than 80%, 90%, 95%, 97% sequence identity, and most preferentially 99% sequence identity to SEQ 9 or SEQ 20. These polynucleotide sequences may include, but by no means limited to, the following sequences: SEQ 9; SEQ 20; SEQ 94; SEQ 95; SEQ 96; SEQ 97; SEQ 98; SEQ 99; and SEQ 100.

According to a preferred embodiment of the present invention, bgl1 genes can be acquired from, but by no means is limited to, the following eukaryotic species: Aspergillus niger, Aspergillus luchuensis, Penicillium vulpinum, Sanghuangporus baumii, Penicillium rolfsii, Trichoderma gamsii, Halenospora varia, Daldinia childiae, Fusarium solani, Glaciozyma antarctica, Monascus purpureus, and Trichophyton interdigitale. These listed organisms are exemplary only and are in no way meant to limit what organisms these genes can be acquired from, nor to limit the scope of the invention.

According to a preferred embodiment of the present invention. SEQ 9 or SEQ 20, upon transcription and translation, provides a β-glucosidase 1 (BGL1) polypeptide sequence SEQ 10. However, according to other preferred embodiments of the invention, polypeptide sequences that are homologous and/or substantially similar to SEQ 10 may also be used in the present invention to produce an alcohol or alcohol precursor. Polypeptide sequences for β-glucosidase 1 (BGL1) in these embodiments will, preferably, have at least 70% sequence coverage, or more preferentially greater than 80%, 90%, 95%, 98%, or most preferentially greater than 99% sequence coverage to SEQ 10, and a sequence identity of, preferably, at least 35% to SEQ 10, or more preferentially greater than 40%, 45%, 50%, 55%, 60%, 65%, 70% 75%, 80%, 85%, 90%, 95%, 97%, or most preferentially greater than 99% sequence identity to SEQ 10. These polypeptide sequences may include, but are not limited to, the following sequences: SEQ 10; SEQ 101; SEQ 102; SEQ 103; SEQ 104; SEQ 105; SEQ 106; SEQ 107; SEQ 108; SEQ 109; SEQ 110; SEQ 111; SEQ 112; SEQ 113; SEQ 114; SEQ 115; SEQ 116; SEQ 117; SEQ 118; and SEQ 119.

In a preferred embodiment of the present invention, the exoglucanase (cex-like) polynucleotide sequence utilized is SEQ 7 or SEQ 147. However, in other embodiments of the invention, polynucleotide sequences that are homologous and/or substantially similar to SEQ 7 or SEQ 147 may also be used. In another embodiment of the present invention, polynucleotide sequences for exoglucanase (cex-like) in these embodiments will, preferably, have at least 70% sequence coverage, or more preferably greater than 80%. 90%, 95%, 98%, or most preferentially greater than 99% sequence coverage to SEQ 7 or SEQ 147, and sequence identities of at least 70%, or more preferentially greater than 80%, 90%, 95%, 97% sequence identity, and most preferentially 99% sequence identity to SEQ 7 or SEQ 147. These polynucleotide sequences may include, but by no means limited to, the following sequences: SEQ 7; SEQ 120; SEQ 121; SEQ 122; SEQ 123; SEQ 124; SEQ 125; SEQ 126; SEQ 127; and SEQ 147

According to a preferred embodiment of the present invention, cex-like genes can be acquired from. but by no means is limited to, the following prokaryotic species: Cellulomonas uda, Cellulomonas palmilytica, Cellulomonas cellasea, Saccharothrix syringae, Promicromonospora iranensis, Glycomyces paridis, Micromonospora fulviviridis, Couchioplanes caeruleus, Streptomonospora alba, Phytoactinopolyspora halotolerans, Cellulomonas wangsupingiae, Xylanimonas cellulosilytica, Actinoplanes lutulentus, or Micromonospora saelicesensis. These listed organisms are exemplary only and are in no way meant to limit what organisms these genes can be acquired from, nor to limit the scope of the invention.

According to a preferred embodiment of the present invention, SEQ 7 or SEQ 147, upon transcription and translation, provides an exoglucanase (CEX-like) polypeptide sequence SEQ 8. However, according to other preferred embodiments of the invention, polypeptide sequences that are homologous and/or substantially similar to SEQ 8 may also be used in the present invention to produce an alcohol or alcohol precursor. Polypeptide sequences for exoglucanase (CEX-like) in these embodiments will, preferably, have at least 70% sequence coverage, or more preferentially greater than 80%, 90%, 95%, 98%, or most preferentially greater than 99% sequence coverage to SEQ 8, and a sequence identity of, preferably, at least 35% to SEQ 8, or more preferentially greater than 40%, 45%, 50%, 55%, 60%, 65%, 70% 75%, 80%, 85%, 90%, 95%, 97%, or most preferentially greater than 99% sequence identity to SEQ 8. These polypeptide sequences may include, but are not limited to, the following sequences: SEQ 8; SEQ 128; SEQ 129; SEQ 130; SEQ 131; SEQ 132; SEQ 133; SEQ 134; SEQ 135; SEQ 136; SEQ 137; SEQ 138; SEQ 139; SEQ 140; SEQ 141; SEQ 142; SEQ 143; SEQ 144; SEQ 145; and SEQ 146.

According to another aspect of the present invention, ethanologenic cells were genetically modified by inserting a bacterial promoter sequence into the bacterial genome, immediately followed by a cellulose hydrolytic gene(s) related to the degradation of cellulose. In some embodiments, the native promoter and polynucleotide sequence could also include the promoter and ribosomal binding site (RBS) including various combinations from the following genes: Glyceraldehyde-3-phosphate dehydrogenase, type I (gap) (Pgap), Thiamine pyrophosphate protein TPP binding domain-containing protein (pdc) (Ppdc), the EF-Tu transcription factor (Ptuf), Phosphopyruvate hydratase (eno) (Peno), 2-Dehydro-3-deoxyphosphogluconate aldolase/4-hydroxy-2-oxoglutarate aldolase (eda) (Peda), glucose-6-phosphate dehydrogenase (zwf) (Pzwf), ROK family protein (frk) (Pfrk), Carboxymethylenebutenolidase (clcD1) (PeleD1), as well as Glucosamine-fructose-6-phosphate aminotransferase (glmS) (PglmS), however this list of possible promoters is in no way meant to limit the scope of the invention. In preferred embodiments, the promoter and RBS polynucleotide sequence to regulate cellulose degradation for the production of an alcohol or alcohol precursor is the native promoter for the Glyceraldehyde-3-phosphate dehydrogenase, type I (gap) gene (Pgap), obtained from Z. mobilis. Preferably, said organism further comprises a promoter and RBS sequence which drives gene expression, wherein the genetic material for the promoter/RBS sequences comprises at least one or another of the following: SEQ 5; SEQ 6; SEQ 7; SEQ 8; SEQ 9; SEQ 10; SEQ 11; SEQ 12; and SEQ 13.

In some embodiments of the present invention, other “growth-promoting agents” may be added to the incubation mixture to promote the growth and/or the expression of the genes necessary for the production of alcohols and/or alcohol precursors. These “growth-promoting agents” may be selected from the group comprising nitrogen-based growth-promoting agents, phosphorus-based growth promoting, and other growth promoting agents, and combinations thereof. In a preferred embodiment of the present invention, the nitrogen-based growth-promoting agents are selected from the group consisting: ammonia, inorganic and organic ammonium salts, nitrite and nitrate salts, urea, amines and amino acids, peptides, extracts from animal and plant-based industries (i.e., meat and/or yeast extract, soybean meal, corn meal, corn flour, soybean flour, gelatin, collagen, etc.), and combinations thereof. In a preferred embodiment of the present invention, the phosphorus-based growth-promoting agents are selected from the group comprising: phosphate salts including but not limited to phosphate salts of Groups I and II, hydrogen phosphate salts of Groups I and II, dihydrogen phosphate salts of Groups I and II, and combinations thereof. In another preferred embodiment of the present invention, the other growth promoting agents are selected from the group consisting of: vitamins, inorganic salts of transition and non-transition metals, heavy metal-containing salts, and/or combinations thereof. Some further examples include, but are not limited to, Vitamin B2, Vitamin B1, Vitamin B5, Vitamin B7, sodium chloride, iron sulfate, magnesium sulfate, manganese sulfate, calcium carbonate, sodium hydroxide, etc.

It is known to the person skilled in the art that a plethora of different techniques can be utilized for the extraction and purification of the desired alcohol or alcohol precursor from the cells or the mixture in which the cells are. These methods include, but are not limited to centrifugation, filtration, dialysis, crystallization, ion exchange, electrodialysis, solvent extraction, evaporation, liquid-liquid extraction, distillation, and nanofiltration.

Within the context of this disclosure, it is understood that the person skilled in the art knows that a polynucleotide is defined as the collection of individual nucleotides in any organization or size that relates to the DNA sequence. The term expression within this invention refers to the generation of a polypeptide sequence which is produced based on its polynucleotide sequence or gene. The term gene references a DNA sequence that encodes for a specific polypeptide sequence. A gene can include both sequences between coding regions (introns) and the encoding sequence itself (exon). Genetic modification or related statements herein refer to the alteration of the genetic code of an organism which includes the insertion or deletion of DNA sequences within an organism. Within the context of this disclosure, it is understood that the person skilled in the art knows that a genetic modification can include insertion and maintenance of an expression vector into the organism, or the direct modification of the organism's genome by directly adding or deleting genes through processes like, but not limited to, 2 step allelic exchange or CRISPR cloning. Within the context of this disclosure, it is understood that the person skilled in the art knows that the term enzyme within the present invention defines a polypeptide sequence, specifically in the form of a protein, that can modify a biological molecule or take part within its generation through direct or indirect interactions. The process by which an enzyme influences the modification and/or production of a biological molecule and/or product is termed enzymatic activity.

While the foregoing invention has been described in some detail for purposes of clarity and understanding, it will be appreciated by the person skilled in the art, once they have been made familiar with this disclosure, that various changes in form and detail can be made without departing from the true scope of the invention in the appended claims.

1. A method of producing an alcohol or alcohol precursor from a cellulosic material and a genetically modified live ethanologenic organism, wherein the method comprises the steps of:
exposing said genetically modified live ethanologenic organism to a culture media with a pH of between 2 and 9 thereby creating an incubation mixture;
exposing said incubation mixture to a source of cellulose;
incubating said genetically modified live ethanologenic organism in said incubation mixture with said source of cellulose under aerobic and/or anaerobic conditions at a temperature ranging from 0° C. to 60° C. for a first period of time sufficient for the degradation of cellulose, thereby creating a degraded cellulose mixture;
optionally, incubating said genetically modified live ethanologenic organism in said degraded cellulose mixture under aerobic and/or anaerobic conditions at a temperature ranging from 0° C. to 60° C. for a second period of time sufficient for the production of said alcohol or alcohol precursor; and
optionally, recovering said alcohol or alcohol precursor from the cells and/or spent culture media.
wherein said genetically modified live ethanologenic organism comprises at least one of the following polynucleotide sequences in its genome:
i. at least one endoglucanase (cen-like) polynucleotide sequence selected from the group consisting of an endoglucanase A-like (cenA-like) polynucleotide sequence, an endoglucanase B-like (cenB-like) polynucleotide sequence and an endoglucanase C-like (cenC-like) polynucleotide sequence;
ii. an exoglucanase (cex-like) polynucleotide sequence; and
iii. a β-glucosidase 1 (bgl1) polynucleotide sequence;
wherein said source of cellulose has a lignin content of at most 1 wt. % and a hemicellulose content of at most 15 wt. %, and wherein said live ethanologenic organism belongs to a genus selected from the group consisting of: Aspergillus, Mucor, Zymomonas, Escherichia, Clostridia, Bacillus, and Pseudomonas.

2. A method to obtain an alcohol or alcohol precursor from a lignocellulosic biomass and a genetically modified live ethanologenic organism, wherein said process comprising the steps of:
providing a lignocellulosic biomass;
exposing said lignocellulosic biomass to a modified Caro's acid composition for a period of time necessary to remove more than 98.5% of the lignin present in said lignocellulosic biomass and thus obtaining a source of cellulose and a liquid stream;
separating said solid stream comprising said source of cellulose from said liquid stream comprising said lignin;
exposing said source of cellulose to said genetically modified live ethanologenic organism in a culture media with a pH of between 2 and 9, thereby creating an incubation mixture;
incubating said genetically modified live ethanologenic organism in said incubation mixture with said source of cellulose under aerobic and/or anaerobic conditions at a temperature ranging from 0° C. to 60° C. for a first period of time sufficient for the degradation of cellulose, thereby creating a degraded cellulose mixture;
optionally, incubating said genetically modified live ethanologenic organism in said degraded cellulose mixture under aerobic and/or anaerobic conditions at a temperature ranging from 0° C. to 60° C. for a second period of time sufficient for the production of said alcohol or alcohol precursor; and
optionally, recovering said alcohol or alcohol precursor from the cells and/or spent culture media;
wherein said genetically modified live ethanologenic organism comprises at least one of the following polynucleotide sequences in its genome:
i. at least one endoglucanase (cen-like) polynucleotide sequence selected from the group consisting of an endoglucanase A-like (cenA-like) polynucleotide sequence, an endoglucanase B-like (cenB-like) polynucleotide sequence and an endoglucanase C-like (cenC-like) polynucleotide sequence;
ii. an exoglucanase (cex-like) polynucleotide sequence; and
iii. a β-glucosidase 1 (bgl1) polynucleotide sequence;
wherein said live ethanologenic organism belongs to a genus is selected from the group consisting of: Aspergillus, Mucor, Zymomonas, Escherichia, Clostridia, Bacillus, and Pseudomonas.

3. The method according to claim 2, where said modified Caro's acid is selected from the group consisting of: composition A; composition B; composition C; composition D; composition E; composition F; composition G; composition H; composition I; and composition J; wherein said composition A comprises:
sulfuric acid;
a compound comprising an amine moiety and a sulfonic acid moiety selected from the group consisting of: taurine; taurolidine; taurocholic acid;
tauroselcholic acid; tauromustine; 5-taurinomethyluridine and 5-taurinomethyl-2-thiouridine; homotaurine (tramiprosate); acamprosate; taurates; aminoalkylsulfonic acids where the alkyl is selected from the group consisting of C₁-C₅ linear alkyl and C₃-C₅ branched alkyl; and
a peroxide; and wherein sulfuric acid, said compound comprising an amine moiety and a sulfonic acid moiety and said peroxide are present in a molar ratio of no less than 1:1:1;
wherein said composition B comprises:
sulfuric acid;
a compound comprising an amine moiety;
a compound comprising a sulfonic acid moiety; and
a peroxide; wherein sulfuric acid and said a compound comprising an amine moiety and said compound comprising a sulfonic acid moiety are present in a molar ratio of no less than 1:1:1;
wherein said composition C comprises:
an alkylsulfonic acid; and
a peroxide; wherein said alkylsulfonic acid and said peroxide are present in a molar ratio of no less than 1:1;
wherein said composition D comprises:
sulfuric acid;
a heterocyclic compound; and
a peroxide; and wherein sulfuric acid and said a heterocyclic compound; are present in a molar ratio of no less than 1:1;
wherein said composition E comprises:
sulfuric acid;
a modifying agent comprising a compound containing an amine group; and
a peroxide; and wherein sulfuric acid and said compound containing an amine group; are present in a molar ratio of no less than 1:1;
wherein said composition F comprises:
sulfuric acid;
a modifying agent comprising an alkanesulfonic acid and
a peroxide; and wherein sulfuric acid and said alkanesulfonic acid are present in a molar ratio of no less than 1:1;
wherein said composition G comprises:
sulfuric acid;
a substituted aromatic compound; and
a peroxide; and wherein sulfuric acid and said substituted aromatic compound; are present in a molar ratio of no less than 1:1;
wherein said composition H comprises:
sulfuric acid;
a modifying agent comprising an arylsulfonic acid;
a peroxide; and
optionally, a compound containing an amine group; wherein sulfuric acid and said a arylsulfonic acid; are present in a molar ratio of no less than 1:1;
wherein said composition I comprises:
sulfuric acid;
a heterocyclic compound;
an alkanesulfonic acid and
a peroxide; and wherein sulfuric acid and said a heterocyclic compound; are present in a molar ratio of no less than 1:1;
wherein said composition J comprises:
sulfuric acid;
a carbonyl-containing nitrogenous base compound; and
a peroxide;
wherein sulfuric acid and said a carbonyl-containing nitrogenous base compound; are present in a molar ratio of no less than 1:1.

4. The method according to claim 2, wherein said delignification step is carried out at a temperature ranging from 40 to 55° C.

5. The method according to claim 1 further comprising a step of re-exposing said live ethanologenic organism to unused or spent media for the continuous production of said alcohol or alcohol precursor from said source of cellulose.

6. The method according to claim 1, where said alcohol or alcohol precursor is selected from the group consisting of: polysaccharides;
oligosaccharides; di-saccharides; monosaccharides; organic acids and their corresponding salts, aldehydes, alcohols, ketones, etc.

7. The method according to claim 1, where said alcohol or alcohol precursor is selected from the group consisting of: cellobiose, glucose, and ethanol.

8. The method according to claim 1, where said alcohol or alcohol precursor is ethanol.

9. A method of growing a genetically modified live ethanologenic organism, wherein said method comprises the steps of:
exposing said live ethanologenic organism to a culture media with a pH of between 2 and 9 comprising a carbon source and a nitrogen source, thereby creating an incubation mixture; and
incubating said live ethanologenic organism in said incubation mixture under aerobic and/or anaerobic conditions at a temperature ranging from 0° C. to 60° C. for a period of time sufficient to allow for the exponential growth of said live ethanologenic organism.
and wherein said live ethanologenic organism comprises at least one of the following polynucleotide sequences in its genome:
i. an endoglucanase (cen-like) polynucleotide sequence selected from the group consisting of an endoglucanase A-like (cenA-like) polynucleotide sequence, an endoglucanase B-like (cenB-like) polynucleotide sequence and an endoglucanase C-like (cenC-like) polynucleotide sequence;
ii. an exoglucanase (cex-like) polynucleotide sequence; and
iii. a β-glucosidase 1 (bgl1) polynucleotide sequence.
and wherein said live ethanologenic organism belongs to a genus is selected from the group consisting of: Aspergillus, Mucor, Zymomonas, Escherichia, Clostridia, Bacillus, and Pseudomonas.

10. The method according to claim 9 wherein said carbon source is selected from the group consisting of: cellulose; hydrolysates and other complex sugar-based mixtures, long-chain saccharides, short chain saccharides, monosaccharides, disaccharides, sugar alcohols, organic acids and their corresponding salts and/or combinations thereof.

11. The method according to claim 9 wherein said carbon source is cellulose

12. The method according to claim 10 wherein said nitrogen source is selected from the group consisting of: nitrogen, amines and amino acids, oligopeptides, polypeptides, extracts from animal and plant-based industries, peptone, tryptone, nitrogen-containing vitamins, urea and compounds of the like, ammonia, nitrite and nitrate salts, inorganic and organic ammonium salts, and combinations thereof.

13. The method according to claim 1, wherein the organism is a prokaryotic organism.

14. The method according to claim 1, wherein the organism belongs to the bacterial genus Zymomonas.

15. The method according to claim 1. wherein the step of incubating said live ethanologenic organism in said incubation mixture occurs under anaerobic conditions.


SEQUENCE LISTING (147 sequences)

SEQ ID NO: 1 - DNA, 1376 bp, Cellulomonas uda
  source 1..1376: mol_type = genomic DNA
atgtccgcccgcagaaccgcgtccgcgctgctgaccgccgcagcgttgtccgtcacaggcctcacggccgtcgcgagcaccgcgagcgcagccgacggctgccgcgtcgactacaccgtccagagccagtggaactccgggttccagggcgacgtcaagatcaccaacctggggtccccgctctcgtcgtggaccgtcgcgtggacgttcccgtcctcgggccagcgcgtctcgcagtcgtggaacgcgaacgtcacgacgagcggctcgcgcgtctccgcgtcgagcatcggctggaacggctcgctcggcacgggtgcgtcgacccagttcggcttcatcggctcgttcaccgactcgaacccggtgccgacgtcgttcacggtcaacggcgtgacgtgcacgggcaccgtcaccccgacgcagggccccacgcagggtccgacccagggccccacgcagggaccgacccagggccccacgcagggtccgacgcagggaccgacccagggccccacgcagaacccgcagccgtcgggtgacctgtacgtcgacaccgagaaccagtcgtacgccgcctggcaggctgcctccggcagcaccaaggacctcctggccaagatcgcgctgaccccccagtcggtgtggatcggcaactggaacagcccgtcggtgtcgcagcagcagatgcgtgacgccacgagccgcgcctcggccgccggcaagacggtgtcggtcgtcatctacgcgatcccgggccgcgactgcggctcgtactcgggcggcggcgtcgccgagagcgagtacgcgcagtggatcgacacggtcgcgagcggcatcgtcggcaagccgatcgtgatcctcgagccagacgcgctcgcccagctcggtgactgcggcggtcagggtgaccgcgtcggcttcctgcggtacgcggcgaagtcgctcacgcaggccggcggccgcgtctacatcgacgcgggccactccggctggctgtcggtggacaccgccgtgaaccgtctgaaccagatcggcttccagtacgcggtgggcttcgcgctcaacacgtcgaactaccagacgacgtccgcgtcgcagcagtacggcgagcagatctcgcagcgcctcggcggcaagggctacgtcatcgacacctcgcgcaacggcaacggctcgaacggcgagtggtgcaacccgcgcggtcgcgcgctcggcgacaagccgcgcctggtgaacgactcgacgggcctcgacgccctgctgtgggtcaagctgccgggtgagtcggacggcacgtgcaacggcggcccggccgccggccagtggtggcaggacatggccctggagctcgcgcgcaacgccaagtggtgacc

SEQ ID NO: 2 - protein, 457 aa, Cellulomonas uda
MSARRTASALLTAAALSVTGLTAVASTASAADGCRVDYTVQSQWNSGFQGDVKITNLGSPLSSWTVAWTFPSSGQRVSQSWNANVTTSGSRVSASSIGWNGSLGTGASTQFGFIGSFTDSNPVPTSFTVNGVTCTGTVTPTQGPTQGPTQGPTQGPTQGPTQGPTQGPTQGPTQNPQPSGDLYVDTENQSYAAWQAASGSTKDLLAKIALTPQSVWIGNWNSPSVSQQQMRDATSRASAAGKTVSVVIYAIPGRDCGSYSGGGVAESEYAQWIDTVASGIVGKPIVILEPDALAQLGDCGGQGDRVGFLRYAAKSLTQAGGRVYIDAGHSGWLSVDTAVNRLNQIGFQYAVGFALNTSNYQTTSASQQYGEQISQRLGGKGYVIDTSRNGNGSNGEWCNPRGRALGDKPRLVNDSTGLDALLWVKLPGESDGTCNGGPAAGQWWQDMALELARNAKW

SEQ ID NO: 3 - DNA, 2901 bp, Cellulomonas uda
  source 1..2901: mol_type = genomic DNA
atgcgtcgccgaacccagtccccctatccatcccgccgggtcaccggcacccaccgcggccgcgggcgcacccgcctcgccgccggtctgagcctcgccgtcgcggcgagcgcgctcgtcgtccccctggcgacgtccgcgagcgccgcccccgcgtacaactacgcggaggccctgcagaagtcgatgttcttctaccaggcccaggcgtccggcgacctgccggcgaactaccccgtctcctggcgcggcgactccggcatgtcggacggctccgacgtcggcgtcgacctcacgggcggctggtacgacgcgggtgaccacgtgaagttcggcttccccatggccttcacgaccacgatgctcgcgtggggcgccgtcgagtcccccgccggctacaccaagtcgggtcagctcgacgagctcaagggcaacctgaagtgggtcaacgactacttcatcaaggcccacaccaagcccaacgagctgtacgtgcaggtgggcggcggcgaggccgaccacaagtggtggggcccggccgaggtcatgacgatggcccgcccggcgtacaagatcaccgcgtcctgcccgggctccgacgccgcggccgagacggccgccgcgatggcgtcgtcgtcgctcgtcttcgcggactccgacccgtcgtacgcggcgacgctcgtcacgcacgccaagcagctgtactcgttcgcggacacgtaccgcggcgcgtactccgactgcgtcaaggacgcgcaggcgtactacaagtcgtggtcgggctaccaggacgagctcgtctggggcgcgtactggctgtacaaggccacgggtgacgcgacgtacctcgccaaggcggaggccgagtacgacaagctcggcaccgagaaccagtcgacgacgaagtccttcaagtggaccgtcgcgtgggacaacaagcagttcgcgacgtacgcgctgctcgccatggagaccggcaagcagaagtacgtcgacgacgcgaaccgctggctcgactactggaccgtcggcgccgaggggcagcggatcgcctactcgccgggcggcatggccgtgctcgactcgtggggcgcgctgcggtacgccgcgaacacctcgttcgtcgcgctcgtctactccgactggatcaccgactcgacgcgcaaggcccgctaccaggacttcggcgtcaagcagatcaactacgcgctgggcgacaacccgcgcaagtcgtcgtacgtcgtgggcttcggcaccaacccgccgacgatgccgcaccaccgcaccgcgcacggctcgtggctcgactcgatcacgacgcccgccgcctcgcgccacgtgctgtacggcgcgctcgtcggcggaccgtccgcggccaacgacgcgtacaaggacgaccggcaggactacgtcgccaacgaggtcgcgaccgactacaacgccggcttcacctcggcgctcgcctacctggtggaccggtacggcggcacggcgctgacgaacttccccgtggccgagacgcccgacggtgacgagatcttcgtcgaggcgcagctcaaccaggcccccggaagcacgttcaccgaggtcaaggccatgatccgcaaccagtcggcgttcccggcacggtcgctcaccaacggcaagctgcgctactggttccgcaccgacggcttcgccgcgtccgacgtgacgctcgcggccaactactccgagtgcggtccgcagtccggcaagggcgtctccgcgggcggcgacctggcgtacgtcgagctgtcgtgcgtcggccagaacatccacccgggcggccagtcgcagcaccgccgcgagatccagttccgcctcaccggcccggccgggtgggacgcgaccgacgacccgtcgttcaccggcctgacccagacgggcctcgccaagacgaagaacatcacgatgtacgacggcaccacgctcatctggggcaccgagcccacgggcacccaggtgccggacgacaccacggcacccacggcgcccggcctgccggtcgcctcggccgtgacgtcgaccagcgcgacgctcacctggcccgcgtcgagcgacgacacgggcgtcgtgggctacgacgtctaccgcgtccagggcacgacgagcacgctcgtcgcctccccgacggccgcgacgacgtcgctcacgggcctgaccgcgggcacggcgtacacgtactccgtcaaggcgcgtgacgccgcgggcaacgtctcggcgtcgtcgccgtcggtcacgttcacgacgacgaccgctccggccgacacgaccgcaccgaccgtcccgggcacgcccgcggcgaccgcggtcacgacgaccggcgcgctgctcacctgggccgcgtcgaccgactccggctccggcgtcgcgaagtacgaggtgctgcgggtgtccggcacgacgagcacgctcgtcggctcgcccaccacgccgtcgttcacgctcacggggctgacgcccgacacctcgtactcgtacgcggtgcgcgcggtcgacgccgcgggcaacacgtccgccgcctccacggccgtcacgttccggacgctcgaggagacgacgacgcccccgggcacgtgcaaggtcacgtacacgcccaacagctggaacaccggcttcaccgcctcgatcaaggtcgcgaacaccggcacgtcgcccctgacgtggtcgctcgcgttcgacctgaccgcgggccagaagctgtccagcggctggagcgcgacgtgggcgcagtcggggacgaccgtgaccgcgaccgggcagtcgtggaacgcgacgctggccccgggcgcgagcaccgagatcggcttccagggcacgcacaacggccagaacccgagcccgacgacgttcaccgtcaacggcacggcctgcagctga

SEQ ID NO: 4 - protein, 966 aa, Cellulomonas uda
MRRRTQSPYPSRRVTGTHRGRGRTRLAAGLSLAVAASALVVPLATSASAAPAYNYAEALQKSMFFYQAQASGDLPANYPVSWRGDSGMSDGSDVGVDLTGGWYDAGDHVKFGFPMAFTTTMLAWGAVESPAGYTKSGQLDELKGNLKWVNDYFIKAHTKPNELYVQVGGGEADHKWWGPAEVMTMARPAYKITASCPGSDAAAETAAAMASSSLVFADSDPSYAATLVTHAKQLYSFADTYRGAYSDCVKDAQAYYKSWSGYQDELVWGAYWLYKATGDATYLAKAEAEYDKLGTENQSTTKSFKWTVAWDNKQFATYALLAMETGKQKYVDDANRWLDYWTVGAEGQRIAYSPGGMAVLDSWGALRYAANTSFVALVYSDWITDSTRKARYQDFGVKQINYALGDNPRKSSYVVGFGTNPPTMPHHRTAHGSWLDSITTPAASRHVLYGALVGGPSAANDAYKDDRQDYVANEVATDYNAGFTSALAYLVDRYGGTALTNFPVAETPDGDEIFVEAQLNQAPGSTFTEVKAMIRNQSAFPARSLTNGKLRYWFRTDGFAASDVTLAANYSECGPQSGKGVSAGGDLAYVELSCVGQNIHPGGQSQHRREIQFRLTGPAGWDATDDPSFTGLTQTGLAKTKNITMYDGTTLIWGTEPTGTQVPDDTTAPTAPGLPVASAVTSTSATLTWPASSDDTGVVGYDVYRVQGTTSTLVASPTAATTSLTGLTAGTAYTYSVKARDAAGNVSASSPSVTFTTTTAPADTTAPTVPGTPAATAVTTTGALLTWAASTDSGSGVAKYEVLRVSGTTSTLVGSPTTPSFTLTGLTPDTSYSYAVRAVDAAGNTSAASTAVTFRTLEETTTPPGTCKVTYTPNSWNTGFTASIKVANTGTSPLTWSLAFDLTAGQKLSSGWSATWAQSGTTVTATGQSWNATLAPGASTEIGFQGTHNGQNPSPTTFTVNGTACS

SEQ ID NO: 5 - DNA, 3279 bp, Cellulomonas uda
  source 1..3279: mol_type = genomic DNA
atgcgtgctgacgtcgcgcttcgaaccggttcgggtgctcggaaccggttccgtacgcctggtttgcgctgtaccgtccccctggcgacgatcgagggtggtgtccggctctcgtggcagcgcccctgtggccatctggcccggcttcgaggagacgacgtggtttcctggtccagtcgccgtgcacgcacggctgtcctttctgcaggcctgacggccgcagccctggtcgccgcgacggtcgtcgcgccggccgccctcgcggatcccgtggggtcgacgcatgacttcgagtccggcgcgcagggctggcacagctacggcggcgactcgacgtcgtccgtcgtcgacggcgagttctgcgtcgtcgtccccacggggacgaaagacccgtggaccgtcgccgcgcagcacgacgggatcacgttcgaggcgggcaccacctacacggtgtccttcaccgcgcacgcgacgcaggacgtctcggtcaacctgcgcgcgggcatcgggtggcccgacgacgtggcgtcgaccgtcgccctgacctcgcaggagcaggagtactcgttcacctggaaccccgagttcagcggcgagggcaacctgtcgttccagctcggcgcgcaggcggccgactacacgttctgcctcgacgacttccagctcgcgggcgacgccgagctgatcccggacaccagcttcgacggtgtgctgcccgaggggtggagcgcggccggctggaccgtgacctccgagccgggcgagggggagccgctgtgcttcgaggtccccgcgggctcggggcagtacgacggcctcgtgctcaacggcctgccgatcgaggaggacgccaactaccgcctcacgttcacggcgagcgcgagcaacgcggcgacgatccgcacgatcgtcggcgagaacgccgtgccgtggcgcgcggcgttcgccgacaacacccagctgtcgaccgagctcacggagcacgagctcgcgttcacctcgcccgtgacgttcccggcgcagtccgacgacccggccgtcggtgtcgggcaggtcgcgctgcagatgggtgcccgcggggacttcacgttctgcatcacgagcctgtcgctcaagaaggtcgcgacgcccccgccgccgtacgagccggacgtgcgcggacccgtgcgcgtcaaccagctcggctacctgcccgacgggccgaagaacgccacggtcgtgagcgacgccaccgagccgctcgcgtggcggctcgaggactcgtcgggcgccaccgtcgcctcgggtgacgcgacgcccgccgggctcgacccctcgtcgcagctcaccgtgcagacgatcgacttctcgtcgttccgcacggccggcagcgggtacacgctcgtcgtgggcgaggaccgcagcgacccgttcgcgatcgcgaaggacctgtacgcgcagctgcgctacgacgcgctgaactacttctaccccgtgcgctcgggcatcgcggtggacgtccccgacgaccgctacgaccgtcccgccggccacgtgtccgggccggacggcgaggtcaacaagggcgacctcgacgtcgcgtgcctgaccgcggccgaggacggtgccgcgtggtcctacggcacctggacgtgcccgaccggctactcgctcgacgtcgtgggcgggtggtacgacgcaggtgaccacggcaagtacgtcgtcaacggcggcatcgccgtcgcgcagctgctgagcatgtacgagcggtcgctgcactcgcccgacgcggcccgcggcgccctcaaggacggcacgctcgacgtgccggccgacgagagcggcaacggcgtgccggacgtgctcgacgaggcccgctgggagctcgagttcttcctccagatgcaggtgccgaccggctccggcatgacggtgtccgcgaccgacaagcgctctctcgacggcctcgtgcaccacaagatccacgacgtcggctggacgggcctcggcctcctgccgtcgcaggacccgcaggagcgccgcctgcaccggccgtcgacggccgcgacgctcaacgtggcggcgaccgccgcgcagggcgcgcgactgttcgcgaagtacgacaaggcgttctcccagcagctcctgagcgtcgcgcgcagcacctacgccgccgcgaagcgggttcccgacctgtacgcgccgcagtccgcggggcagaacggtggcggcccgtacgacgacacggacgtcagcgacgagttctactgggcggccgccgagctctacctcacgacgggtgagaagacgttcgagaccgccgtgcagagcagcccgctcgcgacgagcgacatctgggcccgctccgggttctcctgggggagcgtcgcggcgctcggccggatggacctcgcgaccgtgcccagcaagctgccgacccgcaaggccgtgcaggcgtcggtcgtgaccggggccgagaagatcctcgcctggcagcaggccgaggcgttcggcacgacctacccgggcgacgccgacggctcgtacgtgtggggctcgagctcgtccgtgctgaacaaccaggtcgtgctcgccacggcgtacgacctcacgggtcaggcgcggttcgcgaacgcggtgctggagtcgatggactacctgctcgggcgcaacgcgctgaacatctcctacatcaccgggtacggcaccaagttcgcctcgcaccagcacagccggtggctcgtcccgccgccgccggggacgatcgcgggcggcccgaactcccagagcagcacgtgggacccggtcatgaaccggctctacccgccgggtcacgactgcgcgccccagctctgctacgtcgacgacatcgaggcgtggtcggtcaacgagctcaccatcaactggaacgccccgatgtcctgggtcgcgtcgttcgtcgccgacctcggcgtgagcggggtcccgaccgcgccggtcgtgaccaagcacccgtcggacgtctcggcgaagaagggcacgtccgtggcgctgcgggccaccgcgagcggcaagcccgcgccgtccgtgcagtggcaggtgcgtgacgccaagggtcgctgggtggacgtgcccggcgcccggacgacgaccctgcggatcgtcgtgacgccggcgaccgacggtgcgcagtaccgcgcggtgttccgcaacgcgtccggtaccgccacgaccgacgtcgcacgggtgacggtgctcgccggccccaagggccccggcccgaagggtccgggtcccatcccgtggcccggtcgcgggtga

SEQ ID NO: 6 - protein, 1092 aa, Cellulomonas uda
MRADVALRTGSGARNRFRTPGLRCTVPLATIEGGVRLSWQRPCGHLARLRGDDVVSWSSRRARTAVLSAGLTAAALVAATVVAPAALADPVGSTHDFESGAQGWHSYGGDSTSSVVDGEFCVVVPTGTKDPWTVAAQHDGITFEAGTTYTVSFTAHATQDVSVNLRAGIGWPDDVASTVALTSQEQEYSFTWNPEFSGEGNLSFQLGAQAADYTFCLDDFQLAGDAELIPDTSFDGVLPEGWSAAGWTVTSEPGEGEPLCFEVPAGSGQYDGLVLNGLPIEEDANYRLTFTASASNAATIRTIVGENAVPWRAAFADNTQLSTELTEHELAFTSPVTFPAQSDDPAVGVGQVALQMGARGDFTFCITSLSLKKVATPPPPYEPDVRGPVRVNQLGYLPDGPKNATVVSDATEPLAWRLEDSSGATVASGDATPAGLDPSSQLTVQTIDFSSFRTAGSGYTLVVGEDRSDPFAIAKDLYAQLRYDALNYFYPVRSGIAVDVPDDRYDRPAGHVSGPDGEVNKGDLDVACLTAAEDGAAWSYGTWTCPTGYSLDVVGGWYDAGDHGKYVVNGGIAVAQLLSMYERSLHSPDAARGALKDGTLDVPADESGNGVPDVLDEARWELEFFLQMQVPTGSGMTVSATDKRSLDGLVHHKIHDVGWTGLGLLPSQDPQERRLHRPSTAATLNVAATAAQGARLFAKYDKAFSQQLLSVARSTYAAAKRVPDLYAPQSAGQNGGGPYDDTDVSDEFYWAAAELYLTTGEKTFETAVQSSPLATSDIWARSGFSWGSVAALGRMDLATVPSKLPTRKAVQASVVTGAEKILAWQQAEAFGTTYPGDADGSYVWGSSSSVLNNQVVLATAYDLTGQARFANAVLESMDYLLGRNALNISYITGYGTKFASHQHSRWLVPPPPGTIAGGPNSQSSTWDPVMNRLYPPGHDCAPQLCYVDDIEAWSVNELTINWNAPMSWVASFVADLGVSGVPTAPVVTKHPSDVSAKKGTSVALRATASGKPAPSVQWQVRDAKGRWVDVPGARTTTLRIVVTPATDGAQYRAVFRNASGTATTDVARVTVLAGPKGPGPKGPGPIPWPGRG

SEQ ID NO: 7 - DNA, 1413 bp, Cellulomonas uda
  source 1..1413: mol_type = genomic DNA
atgaccccctcatccatgtccagacgcgcgcgcgtcgcgtccgcgctcgcgatcgtcacgctcggcgcgaccatcgcgacgacgatccccgcccaggccgccggcagcacgctgcaggccgcggcctccgagagcggccggtacttcggtaccgccatcgcggcgttcaagctcaacgacagcacgtactcgtccatcgcgaaccgtgagttcaacatgatcacggccgagaacgagatgaagatggacgcgacggagccgtcgcagaacaacttcagctactccagcggcgaccagatcctcaactgggcgcgcagcaacggcaagcgggttcgtgggcacgcgctcgcgtggcactcgcagcagccgggctggatgcagaacatgtccggcacccagctgcgcaacgcgatgctcaaccacgtcactcaggtcgcgacgcactacaagggcaagatctacgcctgggacgtcgtgaacgaggcgtacgcggacagcggcggcggccgtcgcgactcgaacctgcagcgcaccggcgacgactggatcgaggcggcgttccgcgcggcccgcgccgcggacccgggcgccaagctctgctacaacgactacaacacggacaactggacctgggccaagacgcagggcgtctacaacatggtcaaggacttcaaggcccgtggggtgccgatcgactgcgtcggtttccagtcacacttcaactcgggcagcccgtacccgagcaactaccggaccacgctgcagaacttcgccgcgctcggcgtcgaggtgcagatcaccgagctcgacatcgagggctcgggccagcagcaggcccagacgtacgccaacgtggtcgccgactgcctcgccgtgaaggcctgcaccggcatcacggtgtggggcgtgcgcgactccgactcgtggcgctcctcgggtaccccgctgctgttcgacggctcgggcaacaagaaggccgcgtacacctccacgctcgacgcgctgaaccgcggcggcgtcccgacggacccgacgacgccgcccacggacccgaccacgccgcccacggacccgacgaccccgcccacggacccgaccaccccgccgaccgaccccaccacgcctccgaccgacccgacgggccggtgcacggcgagcctggcgatcgcgaacgcgtggcccggcgggtaccaggcgaccgtgaccgtcaaggcgggctcgtcgtcgatcaacggctggcgcgtcacgctgcccagcggtgtcagcacgagcaacctctggaacggcgtgctcgccaacggtgtggtgaccaacgcgccgtacaacggctcggtcggcgccggccagtcgacgaccttcgggttcgtcggcaacggcagcgctccgagcgcaggctccgtgacctgcgcctga

SEQ ID NO: 8 - protein, 470 aa, Cellulomonas uda
MTPSSMSRRARVASALAIVTLGATIATTIPAQAAGSTLQAAASESGRYFGTAIAAFKLNDSTYSSIANREFNMITAENEMKMDATEPSQNNFSYSSGDQILNWARSNGKRVRGHALAWHSQQPGWMQNMSGTQLRNAMLNHVTQVATHYKGKIYAWDVVNEAYADSGGGRRDSNLQRTGDDWIEAAFRAARAADPGAKLCYNDYNTDNWTWAKTQGVYNMVKDFKARGVPIDCVGFQSHFNSGSPYPSNYRTTLQNFAALGVEVQITELDIEGSGQQQAQTYANVVADCLAVKACTGITVWGVRDSDSWRSSGTPLLFDGSGNKKAAYTSTLDALNRGGVPTDPTTPPTDPTTPPTDPTTPPTDPTTPPTDPTTPPTDPTGRCTASLAIANAWPGGYQATVTVKAGSSSINGWRVTLPSGVSTSNLWNGVLANGVVTNAPYNGSVGAGQSTTFGFVGNGSAPSAGSVTCA

SEQ ID NO: 9 - DNA, 2643 bp, Aspergillus niger
  source 1..2643: mol_type = genomic DNA
atgaggttcactttgatcgaggcggtggctctgactgccgtctcgctggccagcgctgatgaattggcctactcccctccgtattacccctccccttgggccaatggccagggtgactgggcggaagcataccagcgcgctgttgatatcgtctcgcagatgacattggctgagaaggtcaatttgactacgggaactggatgggaattggaattatgtgttggtcagactggaggtgttccccgattgggaattccgggaatgtgtgcacaggatagccctctgggtgttcgtgactccgactacaactctgcgttccccgccggtgtcaacgtggccgcaacctgggacaagaatctggcttacctgcgtggccaggctatgggtcaggagtttagtgacaagggtgctgatatccaattgggtccagctgccggccctctcggtagaagtcccgacggcggtcgtaactgggagggcttctcccccgacccggccctcagtggtgtgctctttgcagagacaatcaagggtattcaggatgctggtgtggttgcaacggctaagcactacatcgcctacgagcaggagcatttccgtcaggcgcctgaagctcaaggctacggattcaatattaccgagagtggaagcgcgaacctcgacgataagactatgcatgagctgtacctctggcccttcgcggatgccatccgtgcaggtgccggtgctgtgatgtgctcgtacaaccagatcaacaacagctatggctgccagaacagctacactctgaacaagctgctcaaggctgagctgggtttccagggctttgtcatgagtgattgggcggctcaccatgccggtgtgagtggtgctttggcgggattggacatgtctatgccgggagacgtcgattacgacagtggcacgtcttactggggtaccaacttgaccatcagtgtgctcaacgggacggtgccccaatggcgtgttgatgacatggctgtccgcatcatggccgcctactacaaggtcggccgtgaccgtctgtggactcctcccaacttcagctcatggaccagagatgaatacggcttcaagtactactatgtctcggagggaccgtatgagaaggtcaaccagttcgtgaatgtgcaacgcaaccatagcgagttgatccgccgtattggagcagacagcacggtgctcctcaagaacgatggcgctcttcccttgactggaaaggagcgcttggtcgcccttatcggagaagatgcgggttccaatccttatggtgccaacggctgcagtgaccgtgggtgcgacaatggaacattggcgatgggctggggaagtggcactgccaactttccctacttggtgacccccgagcaggccatctcgaacgaggtgctcaagaacaagaatggcgtattcactgcgaccgataactgggctattgatcagattgaggcgcttgctaagaccgccagtgtctctcttgtctttgtcaacgccgactctggtgagggttatatcaatgtcgacggaaacctgggtgaccgcaggaacctgaccctgtggaggaacggcgacaatgtgatcaaggctgctgctagcaactgcaacaacacgatcgttattattcactctgtcggcccagtcttggttaacgagtggtacgacaaccccaatgttaccgctattctctggggtggtcttcccggtcaggagtctggcaactccctcgccgacgtgctctacggccgtgtcaaccccggtgccaagtcgcccttcacctggggcaagactcgtgaggcctaccaagattacttgtacaccgagcccaacaacggcaacggagcgccccaggaagacttcgtcgagggcgtcttcattgactaccgcggatttgacaagcgcaacgagactcctatctatgagttcggctatggtctgagctacaccaccttcaactactcgaaccttcaggtggaggttctgagcgcccctgcgtacgagcctgcttcgggcgagactgaggcagcgccgactttcggagaggtcggaaatgcgtcggattacctctaccccgatggactgcagagaatcaccaagttcatctacccctggctcaacagtaccgatcttgaggcgtcttctggggatgctagctatgggcaggatgcctcagactatcttcccgagggagccaccgatggctctgcgcaaccgatcctgcctgccggtggtggtgctggcggcaaccctcgcctgtacgacgagctcatccgcgtgacggtgactatcaagaacaccggcaaggttgcgggtgatgaagttcctcaactggtaagtagacagcaaattcgaaccaagtcggaccaagctaatgaatcgcagtatgtttctcttggcggccctaacgaacccaagatcgtgctgcgtcaattcgagcgtatcacgctgcagccgtcggaagagacgcagtggagcacgactctgacgcgccgtgaccttgcgaactggaatgttgagacgcaggactgggagattacgtcgtatcccaagatggtgtttgtcggaagctcctcgcggaagctgccgctccgggcgtctctgcctactgttcactaagaattcgcc

SEQ ID NO: 10 - protein, 877 aa, Aspergillus niger
MRFTLIEAVALTAVSLASADELAYSPPYYPSPWANGQGDWAEAYQRAVDIVSQMTLAEKVNLTTGTGWELELCVGQTGGVPRLGIPGMCAQDSPLGVRDSDYNSAFPAGVNVAATWDKNLAYLRGQAMGQEFSDKGADIQLGPAAGPLGRSPDGGRNWEGFSPDPALSGVLFAETIKGIQDAGVVATAKHYIAYEQEHFRQAPEAQGYGFNITESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGFKYYYVSEGPYEKVNQFVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVIIHSVGPVLVNEWYDNPNVTAILWGGLPGQESGNSLADVLYGRVNPGAKSPFTWGKTREAYQDYLYTEPNNGNGAPQEDFVEGVFIDYRGFDKRNETPIYEFGYGLSYTTFNYSNLQVEVLSAPAYEPASGETEAAPTFGEVGNASDYLYPDGLQRITKFIYPWLNSTDLEASSGDASYGQDASDYLPEGATDGSAQPILPAGGGAGGNPRLYDELIRVTVTIKNTGKVAGDEVPQLVSRQQIRTKSDQANESQYVSLGGPNEPKIVLRQFERITLQPSEETQWSTTLTRRDLANWNVETQDWEITSYPKMVFVGSSSRKLPLRASLPTVH

SEQ ID NO: 11 - DNA, 250 bp, Zymomonas mobilis
  source 1..250: mol_type = genomic DNA
tcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatatagtagctacttaataagttaggagaataaac

SEQ ID NO: 12 - DNA, 250 bp, Zymomonas mobilis
  source 1..250: mol_type = genomic DNA
ataatcacttaatccagaaacgggcgtttagctttgtccatcatggttgtttatcgctcatgatcgcggcatgttctgatatttttcctctaaaaaagataaaaagtcttttcgcttcggcagaagaggttcatcatgaacaaaaattcggcatttttaaaaatgcctatagctaaatccggaacgacactttagaggtttctgggtcatcctgattcagacatagtgttttgaatatatggagtaagca

SEQ ID NO: 13 - DNA, 250 bp, Zymomonas mobilis
  source 1..250: mol_type = genomic DNA
gcgatggtgccgctggctaacatgttcggctatgtgaaccagctccgttccttcacccagggacgcgcccagtattcgatgcaattttcgcattatgacgaagtcccggctaacgtcgcggatgagttaaaatcgaagatggcttaattaaatactggcataaaccgaaaaatgtcgttatgagcgcgccggagaagcgcggcgcgctcaatacaatagtgataaaagcggtaacaaaaagaggtaacta

SEQ ID NO: 14 - DNA, 250 bp, Zymomonas mobilis
  source 1..250: mol_type = genomic DNA
aatcattggcagctgatgctacattcgccacagttttgttttcggccattgtctatactccagttactcaatacgtaacaataatcagtttatcctaactatagaatcgcatgagaagcgataacgtttcaccataagcaatatattcattgcaacagtggaattgccttatgcgtcaaggaaggatagatcattgacggactgagttcaaaaagagactcgtctaaaagattttaagaaaggtttcgat

SEQ ID NO: 15 - DNA, 250 bp, Zymomonas mobilis
  source 1..250: mol_type = genomic DNA
actatagaatataagttatgttccattcgcagaatagatatagatcagcctctatggatatgctatatatcgcccattccatttaagaataataataaaccatcatgctgtttatttaatatttttattacagtgaattgaagaaatattttcttgataaaaattattaaaaatctatcaccgacgatccgtctctatttcaagatagataataatttgtttaacctgttgattatgcgagataatttta

SEQ ID NO: 16 - DNA, 250 bp, Zymomonas mobilis
  source 1..250: mol_type = genomic DNA
gcagcattaagtatcttaggtggcttgattgttgctcgcttcgtgccggaaaccaaaggtcggagcctggatgaaatcgaggagatgtggcgctcccagaagtagttaaacttgctttggctgaatccttttgtcttttttagataagtcttaaccaattatactttttgtttacaacgatggtataaagcgggcggacaggctaaaaacaggctaaaaggattcggcctctgttttaaggacgagaata

SEQ ID NO: 17 - DNA, 250 bp, Zymomonas mobilis
  source 1..250: mol_type = genomic DNA
ttaaaaaataacttcattttactttaaattttccaagaaaatatttcgaaaatatttttgatatctttcttaattaagaaagaaaacttagttataatcctaccagttggacgaatcgcagacggtcgatttcgatttattcaaaaggccttttggcacagaagaaaaatcgaggtcatcgtcataatttaaagcgaatggacagcatatacctccgtattacggggggattttgtgagtggtgagaata

SEQ ID NO: 18 - DNA, 250 bp, Zymomonas mobilis
  source 1..250: mol_type = genomic DNA
aggcaccaacgcatcaagaacatcagccgccaatcggcctgctttccgcattccggcaaaagcgttttcatcgtaaagtttgatggcatgcgtacgaatagccgcatcaccttctttgacatggatatattcggtcataacttctgctataaccaataagctttccctttgcgagccgcattaacatattccgtttttggattcggaatatctgccgcatcagaggaaagaggaaaaggacggactgaaa

SEQ ID NO: 19 - DNA, 250 bp, Zymomonas mobilis
  source 1..250: mol_type = genomic DNA
gaacgggatcaatatcgctccgatccgatccagaaaatcaaacccttccatcagaatacctcggataaagttacccaattggaaaaactagccgcccttaaaaatcaaggcatcctgacagaagaagaatttgaaaaagaaaaaagacgtattttggccttataaaagacgaaaatatttcttacattgcccccatctcaaagacagtccgcctttcaaaatagatatcacaaaatcgggaaacagaatt

SEQ ID NO: 20 - DNA, 2634 bp, Synthetic construct
  source 1..2634: mol_type = other DNA
atgagattcactttaatcgaagcggttgctcttactgccgtctctttagccagcgctgatgaattggcctattccccgccgtattatccctcaccttgggccaatggccagggtgactgggcagaagcatatcagcgcgctgttgatatagtctctcagatgacattggctgaaaaggtgaatttgacaacggggactggatgggaattggaattatgtgttggtcagacagggggtgttccccgattggggattccgggaatgtgtgcacaggatagccctttgggtgttcgtgattccgattataattctgcgtttccagccggtgtcaatgtggccgcaacctgggacaaaaatctggcttatctgcgcgggcaagcaatgggtcaggagtttagtgataaaggtgctgatatccaattgggtccagctgccggccctctgggtcggagtcccgatggcggtcgtaattgggaaggcttttccccggatccggccttatccggtgtgctgtttgcagaaacaattaagggtattcaagatgctggtgttgttgcaacggctaaacattatattgcctatgagcaggaacattttcgtcaggcgcctgaagctcaaggctatggattcaatattaccgagagtggaagcgcgaacttagacgataaaacgatgcatgagttatatttgtggccattcgcggatgccattcgtgcaggtgccggtgctgtgatgtgctcgtataatcagattaataactcatatggctgtcagaatagctacacactgaataagctgcttaaggcagaattaggtttccagggctttgtcatgagtgattgggcggctcatcatgccggtgtgtcaggtgctttggcagggttggacatgtctatgccgggggacgtcgattatgactcaggcacgtcttattggggtaccaatttgaccatctcagttctgaacgggacggttccgcaatggcgagttgatgatatggctgtacgaatcatggccgcctattacaaagtcggccgtgaccggctgtggacaccgccaaactttagctcatggaccagagatgaatatggcttcaaatattattatgtctcggaaggaccgtatgaaaaggtcaatcagtttgttaatgtgcaacgcaatcatagcgaattgatccgccgtattggagcagacagcacggttctgttgaaaaacgatggggctcttccgttgactggaaaagaacgcttggtcgcccttatcggggaagatgcgggttccaatccttatggtgccaacggctgcagtgatcgtgggtgtgataatggaacattggcgatgggctggggaagtggcacagccaattttccctacttggtgacccccgaacaggccatctccaatgaagttcttaagaataaaaatggcgtattcaccgcaaccgataattgggctattgatcaaattgaggcgttagctaaaaccgccagtgtatctcttgtctttgtcaatgccgactctggtgaaggttatattaatgttgatgggaacctgggtgatcgccgtaacctgaccttatggagaaatggcgataatgtcatcaaggctgctgctagcaactgcaacaatacgatcgttattattcactctgtcggcccagtcttagttaatgaatggtatgacaatcctaatgttaccgctattttatggggtggtttacctggtcaggaatctggcaattcccttgccgacgtgttatatggccgtgtcaacccaggtgccaaatcgcccttcacctggggcaaaactcgtgaagcctaccaagattatttgtataccgagcccaataacggcaacggagcgccacaggaagattttgtcgaaggcgtctttattgattatcgcggatttgataaacggaatgaaacacctatctatgaatttggctatggtctgagctataccaccttcaattattcgaaccttcaggtggaagttttaagcgcccctgcgtacgaacctgcttctggcgaaactgaggcagcgccgacttttggagaagtcggaaatgcgtcggattatctttaccccgatggtctgcaaagaatcaccaaattcatatatccctggttgaactcaaccgatcttgaggcatcttctggggatgctagctatgggcaggatgcctcagactatcttccagagggggccaccgatggctctgcgcaaccgattctgcctgccggtggtggtgctggcggcaatcctcgcctgtatgacgaacttatccgcgttacggttactattaaaaataccggcaaagttgcaggtgatgaagttcctcaactggtatccagacaacaaattcgaaccaaatcggatcaagctaatgaatcgcagtatgtttctcttggcggccctaatgaacccaaaatcgtgctgcgtcaattcgagcggatcacgctgcaaccgtcggaagaaacgcaatggagcacgactctgacgcgccgggatcttgcgaattggaatgttgaaacgcaggattgggaaattacgtcctatccgaagatggtgtttgtcggaagctcctcgcggaaattaccgcttcgggcgtctctgcctacagttcattaa

SEQ ID NO: 21 - DNA, 1365 bp, Neisseria sicca
  source 1..1365: mol_type = genomic DNA
atgtccgcccgcagaaccgcgtccgcgctgctgaccgccgcagcgttgtccgtcacaggcctcacggccgtacgcgagcaccgcgacgcagccgacggctgccgcgtcgactacaccgtccagagccagtggaactccgggttccagggcgacgtcaagatcaccaacctggggtccccgctctcgtcgtggaccgtcgcgtggacgttcccgtcctcgggccagcgcgtctcgcagtcgtggaacgcgaacgtcacgacgagcggctcgcgcgtctccgcgtcgagcatcggctggaacggctcgctcggcacgggtgcgtcgacccagttcggcttcatcggctcgttcaccgactcgaacccggtgccgacgtcgttcacggtcaacggcgtggcgtgcacgggcaccgtcaccccgacgcagggccccacgcagggtccgacccagggccccacgcagggcccgacccagggaccgacccagggtccgacccagggcccgacccagggccccacgcagaacccgcagccgtcgggggacctgtacgtcgacaccgagaaccagtcgtacgccgcctggcaggccgcgtccggcagcaccaaggacctcctggcgaagatcgcgctgaccccgcagtcggtgtggatcggcaactggaacagcccgtcggtgtcgcagcagcagatgcgtgacgccacgagccgcgcctccgccgccggcaagacggtgtcggtcgtcatctacgcgatccctgggcgcgactgcggctcgtactcgggcgggcgcgtcgccgagagcgagtacgcgcagtggatcgacacggtcgcgagcggcatcgtcggcaagccgatcgtgatcctcgagcccgacgcgctcgcccagctcggtaactgcggcggtcagggtgaccgcgtcggctatctgcggtacgcggcgaagtcgctcacagcagccggcggcgtctacatcgacgcgggccactccggctggctgtcggtggacaccgccgtgaaccgtctgaaccagatcggcttccagtacgcgctggcgttcgctctcaacacgtcgaactaccagacgacgtccgcgtcgcagcagtacggcgagcagatctcgcagcgcctcggcggcaagggctacgtcatcgacacctcgcgcaacggcaacggctcgaacggcgagtggtgcaaccgcggtcgcgcgctcggcgacaagccgcgcctggtgaacgactcgacgggcctcgacgccctgctgtgggtcaagctgccgggcgagtcggacggtacgtgcaagcggcccgccgccggccagtggtggcaggacatggccctggagtacgcgcgcaacgccaagtggtga

SEQ ID NO: 22 - DNA, 1350 bp, Cellulomonas sp.
  source 1..1350: mol_type = genomic DNA
atgtccgcccgcagaaccgcgtccgcgctgctgaccgccgcagcgttgtccgtcacaggcctcacggccgtcgcgagcaccgcgagcgcagccgacggctgccgcgtcgactacaccgtccagagccagtggaactccgggttccagggcgacgtcaagatcaccaacctggggtccccgctctcgtcgtggaccgtcgcgtggacgttcccgtcctcggggcagcgcgtctcgcagtcgtggaacgcgaacgtcacgacgagcggctcgcgcgtctccgcgtcgagcatcggctggaacggctcgctcggcacgggtgcgtcgacccagttcggcttcatcggctcgttcaccgactcgaacccggtgccgacgtcgttcacggtcaacggcgtgacgtgcacgggcaccgtcaccccgacgcagggccccacgcagggtccgacccagggcccgacccagggcccgacgcagggaccgacccagggccccacgcagaacccgcagccgtcgggggacctgtacgtcgacacggagaaccagtcgtacgccgcctggcaggccgcgtccggcagcaccaaggacctcctggccaagatcgcgctgaccccccagtcggtgtggatcggcaactggaacagcccgtcggtgtcgcagcagcagatgcgtgacgccacgagccgcgcctcggccgccggcaagacggtctcggtcgtcatctacgcgatcccgggccgcgactgcggctcgtactcgggcggcggcgtcgccgagagcgagtacgcgcagtggatcgacacggtcgcgagcggcatcgtcggcaagccgatcgtgatcctcgagccggacgcgctcgcccagctcggtgactgcggcggtcagggtgaccgcgtcggcttcctgcggtacgcggcgaagtcgctcacgcaggccggcggccgcgtctacatcgacgcgggccactccggctggctgtcggtggacaccgcggtgaaccgtctgaaccagatcggcttccagtacgcggtgggcttcgcgctcaacacgtcgaactaccagacgacgtccgcgtcgcagcagtacggcgagcagatctctcagcgcctcggcggcaagggctacgtcatcgacacctcgcgcaacggcaacggctcgaacggcgagtggtgcaacccgcgcggtcgcgcgctcggcgacaagccgcgcctggtgaacgactcgacgggcctcgacgccctgctgtgggtcaagctgccgggtgagtcggacggcacgtgcaacggcggcccggccgccggccagtggtggcaggacatggccctggagctcgcgcgcaacgccaagtggtga

SEQ ID NO: 23 - DNA, 1371 bp, Cellulomonas palmilytica
  source 1..1371: mol_type = genomic DNA
atgtccgcccgcagaaccgtgtccgcgctgctgtccgtcgcggcgctgtccgtcgcaggcctcacggccgtcgcgagcaccgcgtccgccgccgaaggctgccgtgtcgactacaccgtccagagccagtggaacaccggcttccagggtgacgtgaagatcaccaacctggggtcgccgctgtcgtcgtggacggtcgcctggacgttcccgtcggggagccagaaggtgcagcaggcctggaacgcgaacgtcacgtcgagcggctcgcgcatctccgcgtcgagcgtgggctggaacggttcgctcggcaccggcgcgagcgcgcagttcggcttcatcggcgagtactccggcagcaaccccgtcccgtcgtcgttcacggtcaacggtgtcgcgtgcaccggcaccgtcccgacccaggagccgacgcaggagcccacccaggagcccacgcaggagccgacgcaggagccgacgcaggagcccactgaggagccgacgcaggagcccacgcaggacccgcagccgtccggcgacctgtacgtcgacgtcgagaaccagtcgtacgccgcctggcaggccgcgtccggcagcaccaaggacctcctggcgaagatcgcgctgacgccccagtcggtgtggatcggcaactggaacagcccgtccgtctcgcagcagcagatgcgtgacgcgacgagccgcgcgtcgcaggccggcaagacggtctcggtcgtcatctacgccatcccgggccgtgactgcggctcgcactcgggcggcggcgtcggcgagagcgagtacgcgcagtgggtcgacacggtcgcgcgcggcatcgtgggccgtccgatcgtcatcctcgagcccgacgccctcgcccagctgggtgactgcaacggtcagggcgaccgggtcggcttcctgaagtacgccgccaagtcgctcacgcaggccggcggccgcgtgtacatcgacgcgggccactcgggctggctgtcggtcgacaccgcggtgaaccgcctcaaccagatcggcttcgagtacgcggtgggcttcgcgctcaacacgtcgaactaccagacgacgtccgcgtcgcagcagtacggcgagcagatctcgcagcgcctcggcggcaagggctacgtcatcgacacgtcgcgcaacggcaacggcagcaacggcgagtggtgcaacccgcgcggccgtgcgctgggcgacaagccgcgtctcgtgaacgactcgacgggcctcgacgccctgctgtgggtcaagctgccgggcgagtccgacggctcctgcaacggcggcccggccgccggccagtggtggcaggagatcgccctggagctcgcgcgcaacgccaagtggtga

SEQ ID NO: 24 - DNA, 1404 bp, Cellulomonas xiejunii
  source 1..1404: mol_type = genomic DNA
atgcccccccgtacgaaggcggcccccgccgtcggacgcaagatcgtcgcggcgctcgccgcaagcgcggtcgtcgccgtcggcgtcaccgctctcacctccaccgcggcgaacgccgccgcaggttgccgcgcggactacaccgtcaccaaccagtggcccggcggcttcggcgcgtcggtcaccgtcaccaacctcggcgacccgctgtcgtcgtggaacctgcgctggacgttccccaacggccagagcatccagtcgctgtggaacggccaggtgtcgtcgagcggctcgcagatcacggtcaccaactcgccgtggaacggctcggtcggcaccaacggtgccatccaggtcgggttcaacggctcctggagcggcagcaacggcgccccgacgtcgttcacgctcaacggcacgacgtgcacgggctcggtcaccggcccgacgcagcagcccacccagcagcccacccagcagccgacgcagcagcccacgcagcagccgacgcagcagccgacgcagcagcccacgcagcagcccacgcagaacccgcagcccagcggggacttctacgtcgacccgaacaccgcggcgtacgccgcgtggcaggccgcgtcgggcaacgacaaggcgctcctggcgaagatcgcgcagacgccccaggcgctgtggatcggcgactgggcgaccgcctcggccacggcggagcaggtccgtgactacacgggccgtgcccgcgccgccggcaagacggcacagatcgtcgtctacgcgatcccgggccgggactgcggcaactactcgggtggcggcgtctcgacgtcggagtacgcgcgctgggtcgacaccgtcgcggcgggcatccagggcaacccgtgggtgatcctggagcccgacgcgctcgcgcagctcggtgactgcagcggccagggcgaccgggtcggcttcctgcagtacgcggccaagtcgctgaccgccaagggcgcccgcgtctacatcgacgcggggaactcggcgtggctctcggcgtcggaggcggcgaaccgcctcaaccgcatcgggttcagcgacgcggtcggcttctcgctgaacgtctcgaactaccggaccaccgcggaggccaaggcctacgggcagcagatctcccagctgaccggcggcaagaagttcgtcatcgacacctcgcgcaacggcaacggtgcgacgggcagcgagtggtgcaaccccagcggtcgcgcgctgggtgagcgtcccacgaaggtgaacgacggcagcgggctcgacgcgctgctgtggatcaagcgtcccggggagtcggacgggacgtgcaacggtggcccgtcggccggcgcctggtggcaggcgatggccctcgagctggcgcgcaacgccaagtggtga

SEQ ID NO: 25 - DNA, 1380 bp, Xylanimonas cellulosilytica
  source 1..1380: mol_type = genomic DNA
atgactacccgacgccatcgtctcgcgaccctggccgccgccgtctcgctggccgcggccggcgccgtcgtgaccgcgaccagcgccagcgcggctcccggctgcgccgtcacctatcaggtcgccaacgcgtggcagggcggcttccagggcgacgtgaccgtggagaacgtgggcgatgccctctcctcgtggaccctggagtggacggccgccgccgggcagggcgtggaccaggcgtggaacgccaccgtgtcgcagtccggcaaccaggtgaccgcgacgaacgtcgcctggaacgggtcgctcgccacgggggcgagcgcgtcgttcggcttcatcgggtcgggcagctcgacgccggtgccgacgtcgttcaccgtcaacggcgtgacgtgcacgggttcggtgacgacgccgaccccgacgcccactccgacacctactcccactccgacgccgacgccgacgccgacgccggaccccacgcctacccccacccccactcccactccgacgccgaccgtgccgccggtcgaggccggcgagctctacgtcgacccgacgacgcaggcgtacgccgcgtgggaggcggcatcgggcgccgacaaggcgctgctggagaagatcgccctgacgccccagtcctactgggtgggcaactgggcggacgccgcgcactcgaaggccgaggtcgtcgactacacctcccgggcggtcgccgcgggcaagaccggcatcctcaccgtctacgccatccccggccgcgactgcggcaaccactccggcggcggcgtcgagaccagcgagtacgccgcctggatcgacacggtggcgtcgggcatccagggccgcccgtacgtcgtcctcgagccggacgccctcccccagctcggtgactgcgacggccagggcgaccgcgtcggctacctgaagtacgcggccgagaagctgaccgccgcgggcggccgcgtgtacatcgacgccggtcactcggggtggatgggcgtgggcaccgccgtcgagcggctgaaccagatcgggttcgaccacgccgtcggcttcgcgctcaacacctcgaactaccagacgacggccgacagcatcagctacggcgagcagatctcgcagcagctcggcggacgcccgtacgtgatcgacacctcgcgcaacggcaacggctccaacggtgagtggtgcaacccgagcgggcgcgccctcggcgagaagccgcgggtcgtgaacgactccacgggcctcgacgccctgctgtggatcaagctgcccggcgagtccgacggttcgtgcaacggcggcccggccgccggccagtggttcggtgcgatggccctcgagctggcacgcaacgcggcgtggtga

SEQ ID NO: 26 - DNA, 1356 bp, Cellulomonas sp.
  source 1..1356: mol_type = genomic DNA
atgtccacccgcagaaccgccgccgcgctcctcgcagccgcggccgtcgccgtcggcggtctcaccgccgtcacggccagcgccgcgcaggccgccgccggctgccgcgtgagctacgccgtgacgaaccagtggcaggacggcttcggcgccaacgtcaccatcaccaacctcggcgacccggtgtcgtcgtggaagctcgactggacctacggcgccggccagaagatcacgcagctctggaacggcaacgccacggtcaacggctcgcaggtctccgtgaccaacgtgtcgtggaacggccagatcggcacgggcggcaccgcgtcgttcggcttcaacggctcgtggagcggatcgaatccggtcccggcgtccttcagcctcaacggcacgacgtgcacgggcggcaccgtcccgacctcgacgccgacggtcaccccgacccagacgcccacgccgaccgtgacgcccacgccgacgcccaccccgacgacgacggtcccgccgcagccctcgggcagcttctacgtcgacacggccaaccagtcctaccgcgcgtggcagtcggcgtcgggcaccgacaagacgctgctcgcgaagatcgccctcacgccgcagtcgtactgggtcggcaactggaccgaggcgtcgaaggcgcaggccgaggtcgccgactacaccggccgtgccgtggccgcgggcaagacgccgacgctcgtcgtctacgcgatcccgggccgtgactgcggctcctactcgggcggcggcgtcgcggagtccgagtacgcgcgctggatcgacacgatcgcgcagggcatcaagggcaacccgatcgtcgtgctcgagcccgacgcgctcgcccagctcggcgactgctccggccagggcgaccgcatcggcttcctcaagtacgccgcgaagtccctcacgctgaagggcgcgaaggtctacatcgacgccggccactcgtcgtggctgtcggtcgacacggcggtcaaccgcctcaaccaggtcggcttcgagtacgccgtgggcttcgcgctcaacacgtcgaactaccagacgaccgccgcgagcaagtcctacggccagcagatctcgcagcgcctcggcggcaagggcttcgtcatcgacacctcgcgcaacggcaacggctcgaacggcgagtggtgcaacccgcgcggccgtgcgctcggcgagaagcccacgtacgtcaacgacggctccggcctcgtcgcgctgctgtgggtcaagctgcccggcgagtccgacggcacgtgcaacggcggcccggccgccggccagtggtggcaggacatggccctggagctggcgcgcaacgccaagtggtga

SEQ ID NO: 27 - DNA, 1476 bp, Cellulomonas flavigena
  source 1..1476: mol_type = genomic DNA
atgcacccccgctcgaagagacccctcaccaccagacgcaaggtcgtcgcggccgtcgcggccggagccgtcctcgccggcggcgtcaccgccctgacctcgagcatcgcgcaggccgccgccggctgccgcgtcgactacgccgtgacgagccagtggcccggtggcttcggtgcagccgtcaccgtcacgaacctcggcgacccgctctcgtcctgggagctgagctggacgttccccgacggccagggcgtgcagcagctctggaacggcgtgcactcgacctccggttcgaacgtcaccgtgaagaacatgtcgtggaacggttcggtcggcaccaacgccagcgtccaggtcggcttcaacggctcctggaacggcgcgaacaacgcgccgacgtccttcacgctcaacggcacctcgtgcaacggtgcggtcggtggcccgacgacggagccgacgcccgagccgaccccggagcccacgcccgagccgacgccggagccgacgcccgagccgacgccggagcccacgcccgagccgacgccggagcccacgcccgagccgaccccggagcccacgcccgagcccacgcccgagcccacgcccgagcccacgatgccgccggtccaggccggtcagttccacgtcgacaccacgaaccagtcgtaccgcgcctggcaggcggccagcggctccgacaaggacctgctggcgaagatcgccctgacgccgcaggcgtactgggtcggcaactggaacgaggcctcgcacgcgcagcaggaggtccgtgacatcacgtcggccgctgcggccgccggcaggaccgccgtgctcgtcgtctacgccatcccgggccgcgactgcggccagcactccagcggcggcgtgtcgacctccgagtacgcgcagtggatcgacacggtcgcccagggcatcgtcggcaacccgtgggtggtcctcgagcccgacgcgctgccgatgctcggcgactgcgacggccagggcgaccgggtcggcttcctcaagtacgccgcgaagtccctgaccgccaagggtgcgcgcgtctacatcgacgccggccactcggcgtggctgtcgccgtcggaggccgcgaaccgcctcaaccagatcgggttcgaggacgccgtgggcttctcgatcaacgtctccaactaccgcacgacggcggagtcgaagacctggggtcagcaggtctcgcagctgaccggtggcaagaagttcgtcatcgacacgtcgcgcaacggcaacggcccgtccgggtcggagtggtgcaacccgagcggccgcgccctcggcgagcgcccgacgctcgtgaacgacggcagcgggctcgacgcgctgctgtggatcaagctgcccggtgagtcggacggcgcctgcaacggcggcccgggcgccggtcagtggtggcagtcgatggcactggagctggcgcgcaacgcgaagtggtga

SEQ ID NO: 28 - protein, 454 aa, Neisseria sicca
MSARRTASALLTAAALSVTGLTAVREHRDAADGCRVDYTVQSQWNSGFQGDVKITNLGSPLSSWTVAWTFPSSGQRVSQSWNANVTTSGSRVSASSIGWNGSLGTGASTQFGFIGSFTDSNPVPTSFTVNGVACTGTVTPTQGPTQGPTQGPTQGPTQGPTQGPTQGPTQGPTQNPQPSGDLYVDTENQSYAAWQAASGSTKDLLAKIALTPQSVWIGNWNSPSVSQQQMRDATSRASAAGKTVSVVIYAIPGRDCGSYSGGRVAESEYAQWIDTVASGIVGKPIVILEPDALAQLGNCGGQGDRVGYLRYAAKSLTAAGGVYIDAGHSGWLSVDTAVNRLNQIGFQYALAFALNTSNYQTTSASQQYGEQISQRLGGKGYVIDTSRNGNGSNGEWCNRGRALGDKPRLVNDSTGLDALLWVKLPGESDGTCKRPAAGQWWQDMALEYARNAKW

SEQ ID NO: 29 - protein, 444 aa, Cellulomonas cellasea
MLSASLLAVTGIGAITATSAQAAAGCKVDYAVTNQWGGGFGANVTVTNLGDPVSSWTVDWAFPSGQSIQQLWNGVPSQSGAAVSVKNTTWNGSVATGGTASFGFNGSWNGANAVPTGFRLNGVACTGAPVSTPRPTQTSTPTPTPTVTTTPTPTPTVTPTTTPVPSGTFWVNPNTQAATAAAAASGETRRLLEKISTNAAGSWIGNWNSGTTSQQEVRTVTSAAAAKGQTALLIIYAIPGRDCGNHSAGGVATSEYAGWIDQVAAGIVGKPWIILEPDALAQLGDCDGQGDRVGYLKYAAQKLTQAGGRVYIDAGNSAWLSPSVAAQRLNQIGWDHAVGFALNTSNYRTTAESRSYGQQISQLTGGKTFVIDTSRNGNGANGTEWCNPSGRALGERPTLVNSGGLDALVWAKLPGESDGACNGGPAAGQWFQAQALELARNAKW

SEQ ID NO: 30 - protein, 468 aa, Paraoerskovia marina
MHRSPTIPPDAQPPRARHGVALVAATALLALAGTTTLAAVTLDADPAAAAGTCEADFDVNNQWDGGFGTNVAVTNTGDPVTSWEVTWEFSGDQQVTQLWNGTVSQSGNTVHVASLPWNGAVGTGGTLSFGFNGSGDGSTATPTAVTLNGAACSTGTDPTPTPTTTTPTPTPTETTPTPTPTETTDPPAADGDLYVDTGNQAYPAWSAASGDDKALLAKIALTPQAYWVGNWDDPEHAQAEVQDYTGRAVAAGQVPQLVIYAIPGRDCGSYSGGGVAESEYADWIDTMAAGIQGNPLVVLEPDALAQLGDCDGQGDRVGYLAYAASSLTDAGAHVYVDAGHSGWLAPSTAADRLLQVGLDDAVGFAVNTSNYQTTADSRAWAEQVSALTGGAGYVIDTSRNGNGSNGQWCNPSGRALGDRPSLVDDSTNLDALLWVKLPGESDGTCNGGPSAGAWWQSMALELARNASW

SEQ ID NO: 31 - protein, 469 aa, Cellulomonas avistercoris
MPPRTKAAPAAGRKLVAALAAGAVVAAGVTALTSTAASAAAGCRADYAISSQWPGGFGGTVTVTNLGDPLSSWDLRWSFPNGQTIQSLWNGQVSTSGSQVTVTNSPWNGSVGTNGTIQVGFNGSWSGSNGAPTSFTLNGTTCTGGVSGPTQQPTSTPSATPRPTQTPTQTPRPTQTPTPQPTQSQPPVSSSDFYVDPTTSAYAAWQAASGNDKNLLAKIALTPQALWIGDWATASATQEQVRDYTGRARAAGKTAQIVVYAIPGRDCGSYSGGGVSTSEYARWIDTVAAGIQGNPWVILEPDALAQLGDCNGQGDRVGFLQYAAKSLTAKGARVYIDAGNSAWLSASEAANRLNRIGFSDAVGFSLNVSNYRTTAEAKAYGQQISQLTGGKKFVIDTSRNGNGATGSEWCNPSGRALGDRPTKVNDGSGLDALLWVKRPGESDGTCNGGPSAGAWWQSMALELARNATW

SEQ ID NO: 32 - protein, 419 aa, Pseudoduganella lutea
MMHTVFRPALATLVLLASGAASAAPTCAATYATRDDWANGFVVDIVVTNTGSGTVSGWNVTWDYGSPVSLASAPWGARVAVAGGRITASDDGGRPTIEPGASVGFGLPLAYAGARPVPGRITVGGVNCTIQAPSPNLYSDPQSAAARWVRDNAGDGRAAEIGPRIASQPAAKWFGGWSGDIGNAVGSYVGAAAAAGRVPVLVAYNIPARDCGQHSVGGAGSAAAYQDWIRAFANAIGPREAVVVLEPDALPQLDCLEAAGKATRLQLFRHAVAQFNERAPRTALYIDIGNSDWLEPAEAASRLLQAGVANAEGFALNVSNYRTDAESNPYGIAVSNALQQQAGLGKPFVVDTSRNGAGPDGREWCDPAGRKIGVPPRVNAVGSQPAMTLWVKAPGEADGCAAAAGTFLPEMAYKMIHGY

SEQ ID NO: 33 - protein, 595 aa, Micromonosporaceae bacterium
MKLNLLRGGRHPRLRRALAASAALLAGGGMVVVAATQASAAAGCRVTYTVNQWNTGFTANLTVTNLGDPINGWTLRWTFSGNQQITQSWNSRFSQSGQQATLTNEAWNGNLGTNASTNPGFNASYSGTNEIPTSFTLNGTPCTGSVTGSPTGGPTSPTSGPTSPTSGPTSPTSGPTSNPPPGTHLDNPYVGARPYVNPDWSAKAAAEPGGSRIANQPTGIWMDRIAAITGTGGARGLREHMDTALQQGANLIQVVIYDLPGRDCSALASNGELGPDEIDRYKTEFIDPIAEIFADPAYRSLRIVAIIEIDSLPNLVTNAGGEAGATEMCRIMKENGNYVKGIQYALSKFHAIPNVYNYIDAAHHAWLGWDSNFGPAADLLASTAAGAVGGYDTVDGFIVNTANYSALTEPYFNINTTFNGVSVRQSKWVDWNQYVDELTFAQAFRQRLTGAFGNDIGMLIDTSRNGWGGPNRPTGPPTSASNIDDFVNQARIDRRIHAGNWCNQSGAGLGERPRATPAAGIDAYVWIKPPGESDGASQPIDNDEGKGFDQMCDPNYGGNARNGNNPTGALPNAPLSGHWFSAQFQELMQNAYPPL

SEQ ID NO: 34 - protein, 361 aa, Actinomycetales bacterium
MAANAARIGVGALAARLGTAAVTAVLLVGCGTTDTPAPSTPTGGQQPGRAAQNASPLAGPPAPGNPLVGRPFFVDGTGPAATQAAQWEADRAADAAELRKIADRPAAQWIGGEGDVTSKVDAVVGRAAAKGQLPLLVAYNIPHRDCGNFSAGGAGSSDEYRAWIHAFAAGVKGRPVIVVLEPDAVAHAVEGCNQEPDRLALLAEAVSVLKSTGKATVYIDAGNPRWITDVKRLATALQQAGVATADGFALNVANFITTTDNVTYGNAVSDALGGKTHFVVDTSRNGAGPVPGNGDINGGPRWCNPPDRLLGQAPTIETGQPRLDALLWVKRPGESDGACRPGEPEAGEWWPDYALSLAKRS

SEQ ID NO: 35 - protein, 361 aa, Cellulomonas chengniuliangii
MRRKTKIVAGAAAGALVMTVAVALVARSAAGAGAPTPAISAVTMSPSPGPTTAPALHPEEAPALVEPAPVKPAPAAGNPLAGVVFYVNPDTQAVRAAATAHGEAKQLLDKIAGTPQAFWVDDWNGLDSAREEVRGYTTRAVAAGAKPVVVVYAIPGRDCGWYSQGGIEHSGYAAWVDAIADSMVGNPWVILEPDALAQLGDCDGQGDRLGYLRYAAKSLTERAGARVYIEIGNPAWLTPGQAAAKLGLVGFEHAAGFAFNTSNFQPTDVSRAWGEAVSAEVGGKPYVIDVSRNGNGGDGADWCNPSGRALGERPRVVGEGGLDALLWVKLPGESDGPCNGGPAAGMWWQEGALELARNAHW

SEQ ID NO: 36 - protein, 456 aa, Cellulomonas palmilytica
MSARRTVSALLSVAALSVAGLTAVASTASAAEGCRVDYTVQSQWNTGFQGDVKITNLGSPLSSWTVAWTFPSGSQKVQQAWNANVTSSGSRISASSVGWNGSLGTGASAQFGFIGEYSGSNPVPSSFTVNGVACTGTVPTQEPTQEPTQEPTQEPTQEPTQEPTEEPTQEPTQDPQPSGDLYVDVENQSYAAWQAASGSTKDLLAKIALTPQSVWIGNWNSPSVSQQQMRDATSRASQAGKTVSVVIYAIPGRDCGSHSGGGVGESEYAQWVDTVARGIVGRPIVILEPDALAQLGDCNGQGDRVGFLKYAAKSLTQAGGRVYIDAGHSGWLSVDTAVNRLNQIGFEYAVGFALNTSNYQTTSASQQYGEQISQRLGGKGYVIDTSRNGNGSNGEWCNPRGRALGDKPRLVNDSTGLDALLWVKLPGESDGSCNGGPAAGQWWQEIALELARNAKW

SEQ ID NO: 37 - protein, 459 aa, Cellulomonas gelida
MTARRTSSRAAALLSAATLAIGGLTVAASSATAAEGCRVDYTVQSQWPGGFQGDVKITNLGSAISSWKVAWTWPASGQKVAQSWNADVQTSGSQVSASSVGWNGALGTGASASFGFIGAFTSANPAPTSFTVNGVACTGTVPTTGPTQNPTPTPTPTPTPTPTPTPTPTPTPTTTPPPQPSGDLYVDTTNQSYTAWQAASGSTKDLLAKIALTPQSVWIGNWNSPSVSQQQMRDATSRASSAGKTVSVVIYAIPGRDCGSHSGGGVPESEYAQWVDTVARGIVGRPIIVLEPDALAQLGDCSGQGDRVGFLKYAAKSLTQAGGRVYIDAGHSTWLSVDTAVNRLNQIGFEYAVGFALNTSNYQTTSSSQSYGEQISQRLGGKGYVIDTSRNGNGSNGEWCNPRGRALGDKPRLVNDSSGLDALLWVKLPGESDGSCNGGPAAGQWWQEIALELARNAKW

SEQ ID NO: 38 - protein, 467 aa, Cellulomonas xiejunii
MPPRTKAAPAVGRKIVAALAASAVVAVGVTALTSTAANAAAGCRADYTVTNQWPGGFGASVTVTNLGDPLSSWNLRWTFPNGQSIQSLWNGQVSSSGSQITVTNSPWNGSVGTNGAIQVGFNGSWSGSNGAPTSFTLNGTTCTGSVTGPTQQPTQQPTQQPTQQPTQQPTQQPTQQPTQQPTQNPQPSGDFYVDPNTAAYAAWQAASGNDKALLAKIAQTPQALWIGDWATASATAEQVRDYTGRARAAGKTAQIVVYAIPGRDCGNYSGGGVSTSEYARWVDTVAAGIQGNPWVILEPDALAQLGDCSGQGDRVGFLQYAAKSLTAKGARVYIDAGNSAWLSASEAANRLNRIGFSDAVGFSLNVSNYRTTAEAKAYGQQISQLTGGKKFVIDTSRNGNGATGSEWCNPSGRALGERPTKVNDGSGLDALLWIKRPGESDGTCNGGPSAGAWWQAMALELARNAKW

SEQ ID NO: 39 - protein, 450 aa, Cellulosimicrobium funkei
MRATSSTRRARAATFVAAVATLATLGATATAGAAIATSGAAAPSAASAACTVVYRITSSWSGGFQADVTVTNLGAARSGWEIAWDLAPGEGISQLWNGTLVRDGGRVTVGDVGWNASLATGGSASFGLVGTAASAPAVPTSFTLDGVACGGDAPPDPTDPPDPTDPPETPGDVTFHVDETNQAWEAWQSASGTDRDLLAKIALTPQSSWVTDADAQVSRAKVAAFTSAAAAEGATPLLTIYAIPGRDCGSHSGGGTAEAAYRSWVQTVASGIVGEPWVVLEPDALAQLGDCSGQGDRVGMLRDAAHILTDAGARVYVDAGHSAWLSPATAAARLQQVGLDHAVGFALNTSNYRTTAESRAYGEQVAALLGGDVSFVVDTSRNGNGSNGEWCNPRGRALGDQPRAVDDGTHLDALLWVKLPGESDGSCNGGPPAGQWWQEVALELARNASW

SEQ ID NO: 40 - protein, 452 aa, Demequina salsinemoris
MTRRVPALAVAIAIALSLLVTVAQPASAASAPCVAQYTVQSEWPGGFTAQVRIRNTSGKALTAWRAAFRFTDGQAISQVWNADTVRASGRAKVRNVSYNSHVAKGAWIDFGFNGTWSGKNTRPVAFALNGEKCKVRAWTATVSGGIATPGATASSGSEASTLAVGATTTVAQSSTKFFTSADTQAYAAYQSASGSQKRLLAKIARTPTARWIGDWYSASQAKAETRKYTKAAKRAGRTGVLVIYAIPGRDCGSYSAGGLTPKAYKRWIDAVANGIVGKPWIILEPDAIAQMGDCSGQGDRAGLLRYAARVLTKHGGRVYLDAGHSNWNSVSVTVRRLKQVGFRYAKGFALNTSNYNTTKAEKAYGQAISTKVGNRPFVIDTSRNGKGSNGQWCNPSGRGLGARPGLVKDSSNLKALLWIKAPGESDGSCNGGPSAGVWWQKGALTLARNASW

SEQ ID NO: 41 - protein, 451 aa, Glycomyces dulcitolivorans
MNRRQRLAGVVAAAATALAAALGAGFLLSPNAGAQDDVSAQAQALWTNPNTQAARWVAQNPNDSRTAMIRDRIAETPAGTWFTQTSPTGGIAAQVSAVVDGAAAAGAAPIMVVYNIPNRDCGGASGGGASSHSVYRGWIDQIAAQIDGPAYIVLEPDVIAHDCYGSSERAQINASLAYAAQTLNAADPGVKVYIDSGHSGWLDAPDAASRLQAAGIQYADGFSLNTSNYRTTAESTAYANQVRNIVGTNKGAVIDTSRNGNGPLGDQWCDPSGRAIGEHPTTATGVAGIDAYLWVKLPGEADGCAGAAGQFIPDIAYQLANNAGSGWPGDVEPTDPVTTPTGTGPTSEDPTTGGPSQGDCTVHIDVVSSWGSGWQGKVSMTTDDAVNGWTIGWTWPGSQSIQSSWNAQVTTSGSTVTAKDVGWNAAVGAGQTKELFGFVASGSAADFDVDC

SEQ ID NO: 42 - protein, 342 aa, Streptomyces lacrimifluminis
MVVLLVGGCCVGVLGTGEEDARTAGPGTGASVGSGEYAGGREGAGFEPLWVDPRSPAARQAAEWRLAGRDEDAELMDRIASRPQAAWVTGESPRAEVQTVTMAAARDGRTAVLVAYDIPGRDCGQYSGGGALGGLDYRTWVDGFAAGLGDRGAYVIVEPDAVAQVVAGCAQVDAEERYALLAYAVERIKRQPHARVYLDAGNSGWIPQPARLVEALRSAGVGQADGFALNVSNFQTDQDSSVYGDTLSRALGGKHYVVDTSRNGNGPYSGTDTGIGADAESWCNPPGRALGTPPTTVTGRPRLDAQLWIKRPGESDGTCRGGPPAGQWWSSYALELARNARS

SEQ ID NO: 43 - protein, 493 aa, Cryptosporangium aurantiacum
MTDAPRAGLLSRAGLRAGPYLARHRRGSANGRALTAGVGVAALCSVVVIGGTSLVAANATSEPDHSCAAAYTVRHTWSTGFSAEVAVTNTGEDPVKNWNVNWSFDGGKAVVKGWEGEWQQDDGLVTVRAGEDSVRLEPGKTLTLRFTGWHPQGKAPDAAQFALNGVSCQVGGDKEETPESASPTPSSIALPPGLPASPAPTGKPTKPAAGPLGSLFVDPSGQAVEWVEANSGDPRAAVIRERIAEQPQAHWLAANNPGEVESDVRGYTSRAHSRGKVPVLVAYNMTNRDCGGASAGGADSADGYRTWINNFADGLGSGTSIVILEPDALGHLTTCLSADQQQERLDLLEYAGKTIKQASPKARVFYDVGHSAWIDAGEAASRAKQAGAGQYGDGIALNTSNYNTSSSEVSYGKRVLASLGSGQMVVDTSRNGAGPAGGEWCDPAGRKLGKYPTTATGDPKVAAYLWVKRPGESDGCSGSAGSFIPETAYDLSS

SEQ ID NO: 44 - protein, 687 aa, Nocardiopsis lucentensis
MSTFRRVPDLGPAPRRGLAAMSVLLLGGALAAVTLPTTASAATQCEVDYQVANDWGSGFTANVEIANLGDPVDGWELSWDFDGDQQITNIWNATHTQSGPSVTAANVGHNATIGTGSGVSFGFQGTYGGDNPVPVEFTLNGTVCRGEVDPDPTGEPTDEPTDEPTDEPTDPDPNPGERVDNPYVGADVYVNPIWSANAAAEPGGDAVADEPTGVWLDRISAIEGNDSPTTGDYGLRDHLDEALAQAAGDPLVIQVVIYNLPGRDCAALASNGELGPDEIDRYKNEYIDPIADILADYEDTELRVVTTIEIDSLPNLVTNVGSRPTATPECDEMLANGNYVEGVGYALSTLGAIGNVYNYVDAGHHGWIGWDDNFTASAEVFHEAANAAGATPDDVHGFIANTANYSALEEPYIDVDGVIGGALVRQSAWVDWNRYADELSFAQALRTELVGVGFDENIGMLIDTSRNGWGGDERPTSPSSSTDVDTYVDESRMDRRIHIGNWCNQAGAGLGERPQAAPEPGIDAYVWMKPPGESDGSSEEIPNDEGKGFDRMCDPTYEGNPRNGHSMSGALPNAPVSGHWFPEQFQELLDNAYPPXXAPTRVGERGAAGEAAPRHLDSIRQRCLILWSEGDPCNPPEHRDSSSPSGRPSPSSRRSWFTHCPPRPTRAFVSRTAGWWTRRATTSSSAA

SEQ ID NO: 45 - protein, 449 aa, Cellulomonas sp.
MSARRTASALLTAAALSVTGLTAVASTASAADGCRVDYTVQSQWNSGFQGDVKITNLGSPLSSWTVAWTFPSSGQRVSQSWNANVTTSGSRVSASSIGWNGSLGTGASTQFGFIGSFTDSNPVPTSFTVNGVTCTGTVTPTQGPTQGPTQGPTQGPTQGPTQGPTQNPQPSGDLYVDTENQSYAAWQAASGSTKDLLAKIALTPQSVWIGNWNSPSVSQQQMRDATSRASAAGKTVSVVIYAIPGRDCGSYSGGGVAESEYAQWIDTVASGIVGKPIVILEPDALAQLGDCGGQGDRVGFLRYAAKSLTQAGGRVYIDAGHSGWLSVDTAVNRLNQIGFQYAVGFALNTSNYQTTSASQQYGEQISQRLGGKGYVIDTSRNGNGSNGEWCNPRGRALGDKPRLVNDSTGLDALLWVKLPGESDGTCNGGPAAGQWWQDMALELARNAKW

SEQ ID NO: 46 - DNA, 2901 bp, Cellulomonas flavigena
  source 1..2901: mol_type = genomic DNA
atgcgtcgccgaaccgagtccccccatccgtcccgccgggtcaccggcacccaccgcggccgcgggcgcacccgcctcgccgccggtctgagcctcgccgtcgcggcgagcgcgctcgtcgtccccctggcgacgtccgcgagcgccgcccccgcgtacaactacgccgaggccctgcagaagtcgatgttcttctaccaggcccaggcgtccggcgacctgccggcgaactaccccgtctcctggcgcggcgactcaggcatgtcggacggctccgacgtcggcgtcggcctcacgggcggctggtacgacgcgggtgaccacgtgaagttcggcttcccgatggccttcacgacgacgatgctcgcgtggggcgccgtcgagtcccccgccggctacaccaagtcgggtcagctcgacgagctcaagggcaacctgaagtgggtcaacgactacttcatcaaggcccacaccaagcccaacgagctgtacgtgcaggtgggcggcggcgaggccgaccacaagtggtggggtccggccgaggtcatgacgatggcccgcccggcgtacaagatcacggcgtcctgcccgggctccgacgccgcggccgagacggccgccgcgatggcgtcgtcgtcgctcgtgttcgcagactccgacccgtcgtacgcggcgacgctcgtcacgcacgccaagcagctgtactcgttcgcggacacgtaccgcggcgcgtactccgactgcgtcaaggacgcgcaggcgtactacaagtcgtggtcgggctaccaggacgagctcgtctggggcgcgtactggctgtacaaggccacgggtgacgcgacgtacctcgccaaggcggaagccgagtacgacaagctcggcaccgagaaccagtcgacgacgaagtccttcaagtggaccgtcgcgtgggacaacaagcagttcgcgacgtacgcgctgctcgccatggagaccggcaagcagaagtacgtcgacgacgcgaaccgctggctcgactactggaccgtcggcgcggagggccagaggatcgcgtactcgccgggcggcatggccgtgctcgactcgtggggcgcgctgcggtacgccgcgaacacctcgttcgtcgcgctcgtctactccgactggatcaccgactcgacgcgcaaggcccgctaccaggacttcggcgtcaagcagatcaactacgcgctgggcgacaacccgcgcaagtcgtcgtacgtcgtgggcttcggcaccaacccgccgacgatgccgcaccaccgcaccgcgcacggctcgtggctcgactcgatcacgacgcccgccgcctcgcgccacgtgctgtacggcgcgctcgtcggcggcccgtccgcggccaacgacgcgtacaaggacgaccggcaggactacgtcgccaacgaggtcgcgaccgactacaacgccggcttcacctcggcgctcgcctacctggtggaccggtacggcggcacggcgctgacgaacttccccgtggccgagacgcccgacggtgacgagatcttcgtcgaggcgcagctcaaccaggcccccgggagcacgttcaccgaggtcaaggccatgatccgcaaccagtcggcgttcccggcacggtcgctcatgaacggcaagctgcgctactggttccgcaccgacggcttcgccgcgtccgacgtgacgctcgcggccaactactccgagtgcggtccgcagtccggcaagggcgcctccgcgggcggcgacctggcgtacgtcgagctgtcgtgcgtcggccagaacatccacccgggcggccagtcgcagcaccgccgcgagatccagttccgcctcaccggcccggccgggtgggacgcgaccgacgacccgtcgttcaccggcctgacccagacgggcctcgccaagacgaagaacatcacgatgtacgacggcaccacgctcatctggggcaccgagcccacgggcacccaggtgccggacgacaccacggcacccacggcgcccggcctgccggtcgcctcggccgtgacgtcgaccagcgcgacgctcacctggcccgcgtcgagcgacgacacgggcgtcgtgggctacgacgtctaccgcgtccagggcacgacgagcacgctcgtcgcctccccgacggccgcgacgacgtcgctcacgggcccgaccgcgggcacggcgcacacgtactccgtcaaggcgcgtgacgccgcgggcaacgtctcggcgtcgtcgccgtcggtcacgttcacgacgacgaccgctccggccgacacgaccgcaccgaccgtcccgggcacgcccgcggcgaccgcggtcacgacgaccggcgcgctgctcacctgggccgcgtcgaccgacgccggctccggcgtcgcgaagtacgaggtgctgcgggtgtccggcacgacgagcacgctcgtcggctcgcccaccacgccgtcgttcacgctcacggggctgacgcccgacacgtcgtactcgtacgcggtgcgcgcggtcgacgccgcgggcaacacgtccgccgcctccacggccgtcacgttccggacgctcgaggagacgacgacgcccccgggcacgtgcaaggtcacgtacacgcccaacagctggaacaccggcttcaccgcctcgatcaaggtcgcgaacaccggcacgtcgcccctgacgtggtcgctcgcgttcgacctgaccgcgggccagaagctgtccagcggctggagcgcgacgtgggcgcagtcggggacgaccgtgaccgcgaccgggcagtcgtggaacgcgacgctggccccgggcgcgagcaccgagatcggcttccagggcacgcacaacggccagaacccgagcccgacgacgttcaccgtcaacggcacggcctgcagctga

SEQ ID NO: 47 - DNA, 2901 bp, Cellulomonas palmilytica
  source 1..2901: mol_type = genomic DNA
atgcgtccgcgaacccagtccccccacccatccagcagagccacagccggccgacggacccgcgggcgcacccgcctcgccgccggcgtgagcctcgccgtcgcggccagcgcgctcgtcgtccccctggcgacgtccgcgagcgccgctcccgacttcaactacgccgaggcgctgcagaagtcgatgttcttctaccaggcccaggcgtccggcgacctgccggcgaactaccccgtctcgtggcgcggcgactccggcctgcaggacggctccgacgtcggcgtcgacctcacgggcggctggtacgacgcgggcgaccacgtgaagttcgggttcccgatggccttcacgacgaccatgctcgcgtggggtgccgtcgagtcccccggcggctacacgaagtccgggcagctggacgagctcaagggcaacctgaagtgggtcaacgactacttcatcaaggcccacaccaagcccaacgagctgtacgtgcaggtgggcggcggcgaggccgaccacaagtggtggggtccggccgaggtcatgacgatggcccgcccggcgtacaagatcaccgcgtcctgccccggctcggacgccgccgcggagaccgcggccgcgatggcgtcgtcgtcgctcgtgttcgccgactccgacccgtcgtacgcggccacgctcgtcacgcacgccaagcagctctactcgttcgccgacacctaccgcggcgcgtactccgactgcgtcaaggacgcgcaggcgtactacaagtcgtggtcgggctaccaggacgagctcgtgtggggcgcgtactggctgtacaaggccacgggtgacgcgacgtacctcgcgaaggccgaggccgagtacgacaagctcggcacggagaaccagtcgacgacgaagtccttcaagtggaccgtcgcgtgggacaacaagcagttcgcgacgtacgcgctgctcgccatggagacgggcaagcagaagtacgtcgacgacgcgaaccgctggctcgactactggaccgtcggtgccgagggccagaagatcgcgtactcgccgggcggcatggccgtgctcgactcgtggggtgcgctgcggtacgccgcgaacacctcgttcgtcgcgctcgtgtactccgactggatcaccgactcgacgcgcaaggcccgctaccaggacttcggcgtcaagcagatcaactacgcgctgggcgacaacccccgcaagtcgtcgtacgtcgtgggcttcggcgccaacccgccgaccgcgccgcaccaccgcaccgcgcacggctcgtggctcgactcgatcacgacccccgcggcctcgcgccacgtgctgtacggcgcgctcgtcggcggaccgtccgccgcgaacgacgcgtacaaggacgaccggcaggactacgtggccaacgaggtcgcgaccgactacaacgccggcttcacgtccgcgctcgcctacctggtcgaccggtacggcggcaccccgctggcgagcttccccgtggcggagacgccggacggcgacgagatcttcgtcgaggcgcagctcaaccaggcgcccggctcgacgttcaccgaggtcaaggccatgatccgcaaccagtcggcgttcccggcccggtcgctgaccaacggcaagctgcgctactggttccgcaccgacgggttcgcggcgtccgacgtgacgctcacggccaactactccgagtgcggctcgcagtccggcaagggcgtctccgcgggcggcgacctggcgtacgtcgagctgtcgtgcgccggccagaacatccaccccggcggccagtcgcagcaccggcgcgagctgcagttccggctcaccggccccaccggctgggacgcgaccgacgacccgtcgttcgccggcctgacccagacgggcctggccaagacgaagaacatcacgctgtacgacggcacgaagctcatctggggcaccgagccgaccggcacgcaggtcccggacgacaccacggccccgacggcgcccggcctgccggtcgcctcgtccgtgacgtcgacgagcgccacgctcacgtggtccgccgcgacggacgacacgggcgtcgtcgggtacgacgtctaccgcgtccagggcacgacgagcacgctcgtcgcctccccgacgaccaccacgacgtcgctcaccggcctgaccgccggcacggcgtacacgtacaacgtcaaggcgcgcgacgccgccgggaacgtgtcctcggcctcgccgtcggtcacgttcacgacgacgaccgcaccggccgacacgaccgcgccgaccgtcccgggcacgcccgcggcgtccgcggtcacgacgacgggcgcgaccctgtcctggaccgcgtcgaccgacgcgggctcgggcgtcgcgaagtacgaggtgctgcgggtctccggcacgacgacgacgctcgtcggctccccgacgacgccgacgttcacgctcacgggcctgacgccgaagacgtcgtacacgtacgcggtgcgtgcggtcgacgtcgccggcaacacgtccgccgcctcctcgtcggtcacgttcacgacgctcgaggagacgacgacacccccgggtgcgtgcaccgtcacgtacacgcccaacagctggaacacgggcttcaccgcctcggtgaagatcaccaactccggcacgtcggcgctcacgtgggcgctcgcgttcgacctcacggccgggcagaagctgagctccggctggagcgccgactgggcgcagtccgggaccaccgtcaccgcgaagggcttctcctggaacgcgacgctggctcccggtgcgagcaccgagatcggcttccagggcacgcacggcggccagaaccccagccccacgacgttcaccgtcaacggcgcggtctgcagctga

SEQ ID NO: 48 - DNA, 2886 bp, Cellulomonas gilvus
  source 1..2886: mol_type = genomic DNA
atgtcccccctgcaccttcccgcccgtgcgccgcggcgccgggcacgttcccgcagcgtcgcgggaaccgccggcctcgcgctggccgcaggtctcgtcgtcgcgccgatcgcggggagcgcccacgcggccgacccgtcgttcaactacgccgaggccctgcagaagtcgatgttcttctaccaggcccaggcctcgggcgatctgccggcgagctaccccgtctcgtggcgcggcgactccggcctgaccgacggcgcggacgtgggcaaggacctcacgggcggctggtacgacgcgggcgaccacgtgaagttcggcttcccgatggcgttcaccacgacgatgctcgcgtggggcgcggtcgcgagccccgacggctacgccaaggccgggcagctcgacgagctcgagggcaacctgcgctgggtcaacgactacttcatcaaggcccacaccgctcccaacgagctgtacgtccaggtgggcgacggcgaggcggaccacaagtggtggggccccgccgaggtcatgacgatggcgcgcccgtcgatgaagatcaccgcgtcgtgcccggggtcggacgtcgcgggggagaccgcggcggccatggccgcgtcctcgctcgtcttcgccgaggacgacccggcctacgcggcgaagctcgtcacgcacgccaagcagctgtactcgttcgcggacacctaccgcggcgcgtactccgactgcgtgaccggcgcggcggcgtactacaagtcctggtcgggctaccaggacgagctggtctggggtgcctactggctctacaaggcgacgggtgacgcggcgtacctggccaaggccgaggccgagtacgacaagctcggcaccgagaaccagtcgacgacgcggtcctacaagtggaccgtggcgtgggacaacaagcagttcgccacgtacgcgctgctcgcgatggagacgggcaagcagaagtacgtcgacgacgcgaaccgctggctcgactactggagcaccgggtacaacggcgagcggatccgctactcgccgggcggccaggcggtgctcgactcgtggggctcgctgcggtacgccgccaacacctcgttcgtcgcgctcgtctactcggactggatcaccgacccgacgcgaaaggcgcggtaccacgacttcggcgtgggtcagatcgactacgcgctcggtgacaacccgcgcggttcgtcgtacatggtgggcttcggcgtgaacccgccgaccaagccgcaccaccgcaccgcgcacggctcgtggctcgactcgagcgcgacgcccgtcgcgacgcggcacgtgctgtacggcgcgctcgtcggcggcccgagctcgccgaacgacgcgtacgtcgacgaccgccaggactacgtggccaacgaggtcgcgaccgactacaacgcgggcttcacgtctgcgctggcgtacctggtggaccagtacggcggcacgccgctcgcgagcttcccgaggcccgagacgcccgacggcgacgagatcttcgtcgaggcgcagctcaaccagcccccgggcggcacgttcagcgagatcaaggcgatgatccgcaaccagtcggcgttccccgcgcggtcgctcaagaacgccacggtccgctactggttccgcaccgacggcttcgcggcctcggacgtcacgctcaccgccaactactccgagtgcgggccgcagtccggcaagggcgtcagcgcgggcggcacgctgggctacgtgcagctcacgtgcgtgggccagaacatccaccccgggggccagtcgcagcaccggcgtgagctgcagttccggctcaccggcccggcgggctgggacgcgacgaacgacccgtcgttcgcgggcctgacctccacgggcctggccaagaccaaggcgatcacgctgtacgacggcgggacgctggtgtggggcaccgagccgaccggcaccacgccgggcgacaccacggcaccgaccgtcccgggcacgccggtcgcctccgccgtcacgtccacgggcgccacgctcacgtgggccgcctccaccgacgccggctcgggcgtcgcggcctacgacgtctaccgcgtccagggcacgacgagcacgctcgtcgcctccccgacggccgcgacgacgagcgtcaccgggctcacgccgggcacggcgtactcgtacgtcgtcacggcacgtgacgtggccgggaacgtctcggcggcctcggccgcggtgacgttcacgacgacggccgctcccgtggacacgacggcacccaccgtgccgggcacgccggtcgcgtcgggcatcacgtcctcgggtgcggcgctcacgtgggccgcctcgaccgacgcgggcaccggggtcgcgaagtacgaggtgctgcgggtcgacggctcgagcaccacgctcgtcgcatcgcccacggcacccacgaccacgctcacgggcctggccgcggacaccgcgtactcgtacgtggtgcgcgcggtggacggtgccggcaacgtgtccgcgaccagcacggcggtcacgttccggacgctcgccgggtccaccgacccggtcggcacgtgcgccgtcacctactcggccaacagctggaacaccggcttcacgggctcggtcaagatcacgaacaccggcacgtcgccgctcacgtggtcgctcggcttcacgttcccgtcgggccagaagatctcgcagggctggtccgcgacctggacgcagaccggtgccgtggtcacggccaccgggatgtcctggaacgccacgctcgcaccaggtgcgagcacggagatcggcttccagggcacgcactccggcaccaacacggcacccacgtccttcacggtcaacggcgccacctgcagctga

SEQ ID NO: 49 - DNA, 3165 bp, Cellulomonas sp.
  source 1..3165: mol_type = genomic DNA
atgctcggccatgccacggcgccacgcgcgccccgatcccgcggtcgcaccgcgacggcgggagcctccgccctcgccgtcgccgccggactgctcgtcgccccgctcgcgacctccgcgagcgccgcccccgcgtacaactacgccgaggcgctccagaagtcgatgttcttctaccaggcgcagcgctccggcgacctgcccgacaacttccccgtctcgtggcgcggcgactccggcctcaccgacggcgccgacgtcggcaaggacctcaccggcggctggtacgacgccggcgaccacgtgaagttcgggctgccgatggcgttcagcgcgacgatgctcgcgtggggcgcgatcgacagccccggcgggtacacgaagtccggccagctcgacgagctcaaggacaacctgcgcttcgtcaacgactacttcgtcaaggcgcacaccgccccgaacgagctgtacgtgcaggtcggcgacggcgaggcggaccacaagtggtggggacccgccgaggtcatgacgatggcccgcccgtcgtacaaggtgtcggcgtcctgcccgggctccgacgtcgccgccgagaccgcggcctcgctcgcgtccgcgtcgatcctgttcaagggcgacgacccgacgtacgccgcgacgctgatctcgcacgcgaagcagctctattccttcgctgaccagtaccgcggcagctactccgactgcgtcaccgccgcctcggcgtactacaagtcgtggtccggctaccaggacgagctcgtctggggcgcctactggctctacaaggcgacgggtgacgcgtcgtacctgagcaaggcggagacggagtacgacaagctcggcaccgagaaccagagcaccacgaagtcctacaagtggaccgtggcgtgggacaacaagcagttcggcatgtacgcgctgctcgcgatggagaccggcaagcagaagtacgtcgaggacgcgaaccgctggctcgactactggaccgtcggcgtcaacggccagaaggtgccgtactcgcccggcggcatggccgtcctcgactcgtggggtgcgctgcggtacgccgccaacacgtcgttcgtcgcgctcgtctactccgactggctcaccgacgcgacgcgcaaggcgcggtaccacgacttcggcgtgcgccagatcaactacgcgctcggcgacaacccgcgcaagtcgtcgtacgtcgtcggcttcggcaccaacccgccgaccatgccgcaccaccgcaccgcgcacgggtcgtggctcgactcgatcaccacgcccgccgcatcacgccacgtgctgtacggcgcgctcgtcggcgggcccggctcgcccaacgacgcgtacaccgactcgcggcaggactacgtcgccaacgaggtcgcgaccgactacaacgcgggcttcacgagcgcgctcgcgcggctcgtcgacgagtacggcggcaccgcgctcgcgtcgttcccgacgcgtgaggcgcccgacggcgacgagatcttcgtcgaggcgatgctcaaccagcctcccggcaccacgtacaccgagatcaagggcatgatccgcaaccagtcggcgttcccggcccgctcgctcaagaacggcaagatccgctactggttcacgaccgacggcttcgccgcgtcggacgtcacgctgtcgtcgaactactcggagtgcggcgcgcagtccggcaagggcgtcagcgccggcgggaacctcggctacgtcgagctgtcctgcgtcggccagaacatccaccccggcggccagtcgcagcaccgccgggagatccagttccggctcaccggcccgacgggctggaacgcggcgaacgacccgtcgttcgcgggtctcggctcgacggccctcgcgaagacggccgcgatcacgctctacgacggctcgacgctgatctggggcaaggagccgaccggcaccacgaccgacacgaccgcaccgaccgtgccgggcacccccgtcgcgtcgggcatcacgaccgtcggcgcgtcgctcacctgggcggcgtcgaccgacgccgggtcgggcgtcgccgggtacgagctgtaccgggtccaggggtcgacgcagacgctcgtcgggaccaccaccgcggcggcctacatcctgcgtgacctcacgccgggcacgtcctactcctacgtcgtgaaggcgaaggacgtcgccggcaacgtgtccgccgcgtccgccgcggtcacgttcaccaccgagacgcagggcacgaccgagccgccgaccacgccgggcacgcccgtcgcgtccgcggtcacctcgaccggtgcgaccctcacgtgggccgcgtcgaccggcaccccggcggtcgccggctacgacgtcctgcgcgtccagggcaccaccgcgaccgtcgtcgcgcagaccaccgggacgacgcacacgctcaccggcctgacgcccggcaccgcgtacacctacgccgtgcgggcgaagaacaccgcgggcgacgtctccgcggtctccgcctcggtgacgttcacgacgaccgcaccgccggtcgacaccaccgcgccgaccgtccccgggacgcccgtcgcgtcgaacgtcagcacgacgggcgccacgctcacgtggaccgcgtcgaccgacaccggcggaagcggcctgtcgggctacgaggtgctgcgcgtcagcgggacgacgcagacgctcgtcgcgtcgccgaccaccgcgtcggtcgccctcaccggtctggcgcccgccaccgcgtacacctacgtggtccgggccaaggacggggccggcaacacgtccgcggtgtccgcggccgtcaccttcacgacgctgcccacgtcgacgaccgcctcgtgcaaggtcgcgtacacggcgaactcgtggaacaccggcttcaccggcgcggtgaagctcaccaacacgggcacgaagcccctgacctggaccctcgggttcaccttcccctctggccagcaggtcacgcagggctggagcgccacgtggtcgcagaccgggacgacggtcaccgcgacgggcgcgtcgtggaacgccacgctccagcctggtcagagcgtcgacatcgggttcaacggctcccacaccgggaccaacaccgacccgacgtcgttcaccgtgaacggtgaggtctgcggctga

SEQ ID NO: 50 - DNA, 3153 bp, Cellulomonas fengjieae
  source 1..3153: mol_type = genomic DNA
atgcaccgcccccgaactcgcgtcgcgatcgcgtgcagcaccgtgctcaccgtcgccgccggcctgctcgtcgccccgctcgcggggacggcgaccgccgcggagccggcgttcaactacgccgaggcgctccagaagtcgatgttcttctaccaggcgcagcgggcgggtgacctgcccgccgacaaccccgtctcgtggcggggcgactccgccctgaccgacggcgccgacgtcggcaaggacctcaccggcggctggtacgacgccggcgaccacgtgaagttcggcctgccgatggcgttcagctcgaccatgctcgcgtggggtgccatcgccgcaccgcagggctacacgaaggccggtcagctcgacgagctcaagggcaacctgcgctgggtcaatgactacttcgtcaaggcgcacaccgcacccaacgagctgtacgtgcagatcggcgacggcgaggccgaccacaagtggtggggccccgccgaggtcatgacgatggcgcggccgtcgtacaagatcagcccgtcctgcccgggctccgacgtcgcgggcgagacggcggcgtcgctggcctccgcgtcgatcgtgttcaagtccgaggacccgacgtacgcggcgacgctcctgtcgcacgccaagcagctctactcgttcgcggacacctaccgcggcaagtactccgactgtgtgaccgccgccgccgcgtactacaagtcgtggtccggctaccaggacgagctggtctggggcgcctactggctctacaaggcgaccggcgacgcgacctacctggccaaggcggaggccgagtacaccaagctcagcgtcgagaaccagacgaccacccggtcctacaagtggaccgtcgcgtgggacgacaagagctacgcgatgtacccgctgctggccatggagaccggcaagcagcagtacgtcgacgacgccaaccggtggctcgactactggaccgtcggcgtcaacgggcagcggatcccctactcgcccggcggccaggccgtcctcgactcgtggggctcgctgcggtacgcggcgaacacctcgttcgtcgcgctggtgtactcggactggctgaccgacaccacccgcaaggcccggtaccacgacttcggcgtccggcagatcaactacgcgctcggcgataacccgcgcaagtccagctacgtggtcggcttcggggccaacccgccgcagaacccgcaccaccgcaccgcgcacggctcgtggctcgactcgctgacgcagccggagaagacccgtcacgtcctgtacggcgcgctcgtcggcggacccagcggtccgaacgacgcctacaccgacctgcggtccgactacacggccaacgaggtcgccacggactacaacgccgggttctccagcgcgctcgcgcggctgacccaggagtacggcggcaccccgctggccggcttccccgtcgccgagcggccggacggcgacgagatcttcgtggaggccatgctcaaccagccggccggcggcacgttcaccgaggtcaaggcgatgatccgcaaccagtcggcgttccccgcgcgggcgctcaagaacggcacgctgcgctactggttcacgctcgacgccggtgactcggccgccaacctcaccctctcggccaactacagcgagtgcggcccgcagacggccacgccggtcagcgcaggcggggggctctactacgccgagctcacctgcgtggggcagaacatccaccccggcggtcagtcgcagcaccggcgggagatccagttccgggtcagcggtggttccacgtggaacgccgcgaacgacccgtcgttcgcgggtctgagcaccgccggtctggccaagacgaaggcgatcaccctctacgacggcggccggctcatctggggcgccgagccgacgggcacggtcaccgacacgaccgcgccgaccgtgccgagcaccccggtcgcggccggcatcacctcgaccggtgcctcgctgagctggacgccgtccaccgatgcgggcagcggtgtcgcggggtacgaggtctaccgcgtccaggggaccacctcgacgctggtcgcctcgaccaccagcccgtcgacggccctgtccggcctgacgccgagcaccgcgtactcctacacggtccgggccaaggacgtggccggcaacgtgtccgccgcctcggcggcgggcacgttcaccacggcggagtcgcccgtcgaggtcgtcccgcccggcacacccggcacgcccgtggcctcagcggtgacggcgaccggcgccacactgacctggacgccgtcagcggccggcacctacgcggtggccgggtacgacgtgctccgggtcaccgggaccacgtcgagcgtcgtcggcacggcgaccggcacgacgttcgccgtgaccggcctgacggcggggacggcgtacacgtacgcggtgcgcgccagggacgcggccgggaccacctcgcccgtgtccgcggcggtcacgttcaccacgggcgcggccacggacaccacggctcccagcgtgcccggcaccccggtcgcctcgtcgatcacccagaccggcgcgacgctcacctgggccgcgtcgaccgacgcggggggcagcgggctcgcggggtacgacgtgctgcgggtcaacggcaccacctcgacggtcgtcggcaccgcgacgggcaccacgttcgcgctcaccggtctcacggcgaacaccgcgtacacgtacgcggtgcgcgccaaggacgccgccggcaacgtgtccgccgccggcacaccggtcggcttctcgacgctgccggcgacgtccaccagcagctgcgccgtgacctactcgaccaacagctggaacgtcggcttcacgggcagcgtgcggatcacgaacacgtcgaccgcgccgctgacctggacgctcgggttcgcctttccctccggtcagcgcctgacccaggggtggagcgcgacgtgggcccagacggggtccgccgtcacggccaccggcctcgggtggaacacgacgctggcaccgggggcgagcacggacatcgggttcaacggctcgcacgccgggaccaacaccgcgccgaccgccttcactgtcaacggcacggcctgcacggtctcctga

SEQ ID NO: 51 - DNA, 2397 bp, Cellulomonas sp.
  source 1..2397: mol_type = genomic DNA
atggcccgtcaggcacgcgccacgcgcccgtggtgggcgaccgccgccgccctggccctcgccgccggcgcggtcaccgtgccgctgtcggccgcgagcgcggcacccgcgtacaactacgcggaggcgctgcagaagtcgatgttcttctaccaggcgcagcgctcgggcgacctgccggaggacttcccggtctcgtggcgcggcgactcgggcctgcgcgacggcgcggacgtgggggaggacctgaccggcgggtggtacgacgccggggaccacgtgaagttcggcctgcccatggcgttcaccgccacgatgctcgcgtggggcgcgatcgagagcccggaggggtaccgcaaggccgggcagctcgacgagctcaaggacaacctgcgctgggtcaacgactacttcgtcaaggcgcacacggccccgaacgagctgtacgtgcaggtcggcaagggcgacgacgaccacaagtggtggggcccggccgaggtgatgacgatggcccggccggcgtacaggatcaccgcgtcctgcccgggctcggacgtcgccgccgagaccgcggccgcgatggcctccgcctcggtgctgttcgccgaggacgaccccgcctacgcgcgcacgctcgtctcgcacgcgaagcagctctactccttcgccgacacgtaccggggcaactactccgactgcgtcaccgacgcgcagtcgttctaccgctcgtggtccggctaccaggacgagctcgtctggggcgcgtactggctgtacgaggcgacgggcgacgcgtcctacctggcgaaggcggaggcggagtacgacaagctcggcaccgagaaccagacgacgacgaagaagtacaagtggacggtcgcctgggacgacaagtcgttcggcgcgtacgccctgctggcgatggagacggggaagcagaagtacgtcgacgacgcgaaccgctggctggactactggacggtcggcgtgaacggccagaaggtgccgtactcgccgggcggcatggccgtgctcgactcgtggggcgcgctgcggtacgccgcgaacacgtcgttcgtggcgctcgtgtactcggactggatcaccgacgcgacgcgcaaggcgcggtaccacgacttcggcgtccggcagatccactacgcgctcggcgacaacccgcgcaggtcgtcctacgtggtcgggttcggcgcgaacccgccgcagaacccgcaccaccgcaccgcgcacggctcgtggctggactcgctcacgcagccggccgagacgcggcacgtgctctacggcgccctcgtcggcgggcccggctcgccgaacgacgcgtacaccgacagccggtccgactacgtggcgaacgaggtcgcgaccgactacaacgccggcttcacgagcgcgctcgcgcgcctcaccgaggagtacggcggcaccccgctggcgtcgttcccgcgccccgagacgcccgaccaggacgagctgttcgtcgaggcgcggctcaaccagccggccggtggcacgttcaccgaggtgaaggcggtcatccggaaccggtccgccttcccggcgcggtcgctgcgcgacgcgtcggtccggtactggttcacgctcgacgggtccgacccggcgtcggggttgaccgtctccaccaactacagcgagtgcggcacgcagacgaacgcggtgaagcacgccggcggggacctgtactacgccgagctgcggtgcgcgggccaggacatccacccgggcgggcagtcgcagcaccgccgcgagatccagttccgggtcagcgggggagcggcgtgggacgcgacggacgacccgtcgtcggcgggcctcgtcgcgaccggcgaccccgtgaagacccgtgcgatcacgctgtacgaggccggccgtctggtctggggcaccgagcccgacgggacgtcgaccccgacccccacgccgacggtgacgcccaccgcgacccccaccgccaccgccacggccacccccaccgcgaccgcgaccgcgacgccgaccgttaccgccaccccgacgcccacgcagacgtcgagcccgacgccgaccgggtgcgccgtgcggtactccgcgaacgggtggagcaccggcttcacggcgaccgtgcggctgacgaacaccggcaccgcacccctgacgtggtcgctcgcgttcgacctgaccgccgggcagcggctgcagcagggctggagcgcgacgtggagccagagcgggacgcgcgtgaccgccaccggcgccgcgtggaacacgacactcgcccccggggcgtccgtggagctcgggttcaacgggtcgcacaccggccagaacccggccccgacggcgttcacggtcaacgggacggcctgcaccacgggatga

SEQ ID NO: 52 - protein, 966 aa, Cellulomonas flavigena
MRRRTESPHPSRRVTGTHRGRGRTRLAAGLSLAVAASALVVPLATSASAAPAYNYAEALQKSMFFYQAQASGDLPANYPVSWRGDSGMSDGSDVGVGLTGGWYDAGDHVKFGFPMAFTTTMLAWGAVESPAGYTKSGQLDELKGNLKWVNDYFIKAHTKPNELYVQVGGGEADHKWWGPAEVMTMARPAYKITASCPGSDAAAETAAAMASSSLVFADSDPSYAATLVTHAKQLYSFADTYRGAYSDCVKDAQAYYKSWSGYQDELVWGAYWLYKATGDATYLAKAEAEYDKLGTENQSTTKSFKWTVAWDNKQFATYALLAMETGKQKYVDDANRWLDYWTVGAEGQRIAYSPGGMAVLDSWGALRYAANTSFVALVYSDWITDSTRKARYQDFGVKQINYALGDNPRKSSYVVGFGTNPPTMPHHRTAHGSWLDSITTPAASRHVLYGALVGGPSAANDAYKDDRQDYVANEVATDYNAGFTSALAYLVDRYGGTALTNFPVAETPDGDEIFVEAQLNQAPGSTFTEVKAMIRNQSAFPARSLMNGKLRYWFRTDGFAASDVTLAANYSECGPQSGKGASAGGDLAYVELSCVGQNIHPGGQSQHRREIQFRLTGPAGWDATDDPSFTGLTQTGLAKTKNITMYDGTTLIWGTEPTGTQVPDDTTAPTAPGLPVASAVTSTSATLTWPASSDDTGVVGYDVYRVQGTTSTLVASPTAATTSLTGPTAGTAHTYSVKARDAAGNVSASSPSVTFTTTTAPADTTAPTVPGTPAATAVTTTGALLTWAASTDAGSGVAKYEVLRVSGTTSTLVGSPTTPSFTLTGLTPDTSYSYAVRAVDAAGNTSAASTAVTFRTLEETTTPPGTCKVTYTPNSWNTGFTASIKVANTGTSPLTWSLAFDLTAGQKLSSGWSATWAQSGTTVTATGQSWNATLAPGASTEIGFQGTHNGQNPSPTTFTVNGTACS

SEQ ID NO: 53 - protein, 966 aa, Cellulomonas palmilytica
MRPRTQSPHPSSRATAGRRTRGRTRLAAGVSLAVAASALVVPLATSASAAPDFNYAEALQKSMFFYQAQASGDLPANYPVSWRGDSGLQDGSDVGVDLTGGWYDAGDHVKFGFPMAFTTTMLAWGAVESPGGYTKSGQLDELKGNLKWVNDYFIKAHTKPNELYVQVGGGEADHKWWGPAEVMTMARPAYKITASCPGSDAAAETAAAMASSSLVFADSDPSYAATLVTHAKQLYSFADTYRGAYSDCVKDAQAYYKSWSGYQDELVWGAYWLYKATGDATYLAKAEAEYDKLGTENQSTTKSFKWTVAWDNKQFATYALLAMETGKQKYVDDANRWLDYWTVGAEGQKIAYSPGGMAVLDSWGALRYAANTSFVALVYSDWITDSTRKARYQDFGVKQINYALGDNPRKSSYVVGFGANPPTAPHHRTAHGSWLDSITTPAASRHVLYGALVGGPSAANDAYKDDRQDYVANEVATDYNAGFTSALAYLVDRYGGTPLASFPVAETPDGDEIFVEAQLNQAPGSTFTEVKAMIRNQSAFPARSLTNGKLRYWFRTDGFAASDVTLTANYSECGSQSGKGVSAGGDLAYVELSCAGQNIHPGGQSQHRRELQFRLTGPTGWDATDDPSFAGLTQTGLAKTKNITLYDGTKLIWGTEPTGTQVPDDTTAPTAPGLPVASSVTSTSATLTWSAATDDTGVVGYDVYRVQGTTSTLVASPTTTTTSLTGLTAGTAYTYNVKARDAAGNVSSASPSVTFTTTTAPADTTAPTVPGTPAASAVTTTGATLSWTASTDAGSGVAKYEVLRVSGTTTTLVGSPTTPTFTLTGLTPKTSYTYAVRAVDVAGNTSAASSSVTFTTLEETTTPPGACTVTYTPNSWNTGFTASVKITNSGTSALTWALAFDLTAGQKLSSGWSADWAQSGTTVTAKGFSWNATLAPGASTEIGFQGTHGGQNPSPTTFTVNGAVCS

SEQ ID NO: 54 - protein, 898 aa, Micromonospora sp.
MRHHTRPVPGRYRPPVTDQPWPRRLLAADQSWPRRLLATTDQPWPRRLLATTDQPWPRRLLAAGVALLIGLALAVAAPPSGPASAAPAAFNYAEALQKSLLFYEAQQSGRLPDWNRVSWRGDSALTDGADAGLDLTGGWYDAGDHVKFGFPMAFSATMLAWGAVEYRSGYTASGQLPHLLNNLRFVNDYFIRAHPSANVLYGQVGKGDDDHKWWGPAEVLPMARPAYKIDASCGGADLAGETAAAMAASSMVFRPTDAAYADRLLGHARQLYTFADTVRKSYHECITDATSFYRSWSGWQDELVWAAIWLHRATGEASYLAKAESEYDKLGTENQSTTRSYKWTIAWDNKQFGAYVLLANLTGKQKYVDDANRWLDYWTVGVDGQRVPYSPGGMAVLDSWGALRYAANTSFAALVYSDRTTDTTRKARYHDFAVRQINYALGDNPRNSSYVIGFGANAPRNPHHRTAHGSWWDSQTVPVETRHTLYGALVGGPSAANDAYTDSRSDYVMNEVATDYNAGFTSALVRLTSEYGGTPLANFPVAETPDLDELTVETTVMQAEPRATGLKVMIYNKSAFPARALTDGRFRYYFRPDGTGGVQVTAGYTQGCPSPTTARQLSGDIWYVEVDCTGHTIAPAGQSQHRMEVQFKIGVPEGGTWDPTNDPSYQATAGPNRKVPLYSGATRVWGDEPGPVVPDTTAPSPPGTPVASAVTATGLTLSWAASTDNVGVTGYRVHREAGATDPLVGSSTGATLAVSGLTASTAYQFYVVAVDAAGNTSAASAPVTVTTAPPPVTGACTVGYATNDWSTGFTAAVTITNTGTTAINGWTLRFSFPGGQTVSQGWSATVTQAGTAVTATGLSYNGTLAPGASVSFGFNGTHTGSNPRPTAFTVNGVACALG

SEQ ID NO: 55 - protein, 964 aa, Asanoa ferruginea
MHHVRRLRRARSDQPWSRRVLAAGAALVTGLTLGAVPGPASAAVPATTGVQAAAAAFNYAEALQKSLFFYEAQQSGPKPSWNRVSWRGDSALTDGADVGLDLTGGWFDAGDHVKFGFPMAFSATMLAWGAVEYRDGYVSSGQLPHLLNNLRFVNDYFVKAHPSPNVLYGQIGNGDADHKWWGPAEVMPMARPSYKIDASCGGADLAGETAAAMAASSMVFRPTDAAYADKLLGHAKQLYTFADTVRKSYHDCITDATNFYRSWSGYQDELVWGAIWLYRATGDASYLAKAESGYDLLGNENQTTTKSYKWTIAWDNKQFGAYVLLARLTGKQKYLDDANRWLDYWTVGVNGQRVPYSPGGMAVLDSWGALRYAANTSFVALLHGDYLTDATRKARYQDFALRQINYALGDNPRNSSYVIGFGANSPKNPHHRTAHGSWWDSQTVPEETRHVLYGALVGGPSSANDAYSDTRSDYVMNEVATDYNAGFTSALAALVTKHGGTPLANFPVAETPDRDELTVETTVMQAEPRATGLKAIIYNRSAYPARALTDAKFRYYFRAEGATPVVVTPGYTQGCPSPSTARQVSGDLWYVEVDCTGYTIAPAGQSQHRMEVQFKVGVPEGGTWDPTNDPSYSTVTGPNRNVPLYAGGRRVWGVEPGAPTPDTTAPTAPGRPTASAVTATGATLSWAASTDNVGVTGYDVYREAGATDVLVGSPTAATFAVTGLTAATSYAFYVVAKDAAGNRSAASAPVSVTTTDPPAPDAVAPTAPGQPTASTITTSGVTLSWAASTDNVGVTGYRVYRENGATDLLVGSPTGTTLAVTGLTAATAYTFYVVAVDAAANTSPPSASVTVTTAAPPAAGACTVAYATNDWGSGFTANITITNTGTTTINGWSLGFTFPHSGQKVGQGWSATFTQTGTAVTATNLSYNGALAAGASTGIGFNGTHTGSNPKPTSFTLNGVACTA

SEQ ID NO: 56 - protein, 965 aa, Micromonospora profundi
MRHLTGPSTGHPGRSPVADQPWPRRLLAAGVALLTGLSLAVAAPPAGPASAAPAFNYAEALQKSLLFYEAQQSGRLPDWNRVSWRGDSALTDGADAGLDLTGGWYDAGDHVKFGFPMAFSATMLAWGAVEYRSGYTSSGQLPHLLNNLRFVNDYFIKAHPSANVLYGQVGKGDDDHKWWGPAEVMPMARPAYKIDASCGGADLAGETAAAMAASSMVFRPTDVAYADKLLTHARQLYTFADTVRKSYHECITDATSFYRSWSGWQDELVWGAIWLYRATGDATYLAKAESEYDKLGTENQTTTRSYKWTIAWDNKQFGAYVLLANLTGKQKYVDDANRWLDYWTVGVNGQRVPYSPGGMAVLDSWGALRYAANTSFAALVYSDKTTDTTRKTRYHDFAVRQINYALGDNPRSSSYVIGFGANSPRNPHHRTAHGSWWDSQTVPVETRHTLYGALVGGPSSPNDAYSDSRSDYVMNEVATDYNAGFTSALVRLTSEYGGTPLANFPVAETPDIDELTVETTVMQAEPRATGLKAIVYNKSAFPARALTDGKFRYYFRPDGTGPVQVTAGYTQGCPSPTTAKQFSGDIWYVEVDCTGHTIAPAGQSQHRMEVQFKIGVPEGGTWDPTNDPSYQATAGPNRKVPLYSGTTRVWGDEPGPVVPDTAAPSVPGTPVASAVTSNGLTLTWAASTDTGGSGLAGYEITQSQTGSDALVLRTATTNTLAVTGLLPERTYQFTVRALDGAGNRSASSAAVSVTTPAGPAPDTTAPTAPGTPVASAVTATGLTLSWAASTDNVGVTGYRVYREAGATDALVASPTGTTAAVTGLTASTAYQFYVVAVDAAGNNSAASAPVAVTTAPPPVGGTCKVGYATNDWSTGFTATVTITNTGTTALTGWTVRWSFTGGQTVSQGWSATVSQSGTAVTATNLSYNGALAPGASVSFGFNGNHSGSNPKPTAFTVNNTACTIA

SEQ ID NO: 57 - protein, 857 aa, Catellatospora methionotrophica
MPTHSDGIRPPGRWRRLFAGTCGAVLLSSLAVATTAAPAQAAAPYNYGEALQKSLFFYEAQAAGPKPSWNRVSWRGDSALNDGSGVGVNLTGGWFDAGDHVKFGFPMAATATMLAWGAVEYRSAYAASGQLTPLLNNLRFVNDYFIKAHPSANVLYGQVGNGGTDHQWWGPAEVMKMARPAYKIDSSCGGTDLAAETAAAMAAASMVFRPTDATYANTLLSHARQLYTFADTVRKKYSECITDASSYYNSWSGFNDELVWGAIWLHRATGEAAYLAKAESYYANLGTEPQSSTHSYKWTHDWDDKTYGSYVLLAKLTGKQQYVDDANRWLDYWTVGVNGQRITYSPGGQAWLQQWGSLRYSANTAWVAFVYSDWITDATRKARYHDFAARQINYILGDNPNNRSYVNGFGTNPPKNPHHRTAHGSWTDSLTSPVNNRHVLYGALVGGPGSANDAYTDDRQNYQMNEVATDYNGGFTSALVRMYGEYGGTPLASFPPAETPDDAELYVESGLNTTGTTFTEVKALVYNKSAWPARALSNGSFRYYFTLDGSTTPSQISTSVAYSQCSPPTGPTQHSGNVYYVTVSCAGQNIIPAGQSDWRREVQFRITSSGTWDVTNDWSYQATMARNSKITLYDGTTKVWGDAPGGGTPDTTAPSTPGTPSASSITSTSATLTWTASTDTGGSGLAGYRVYREAGTTDVLVGSPATNTLALTGLTASTSYTYYVVALDGAGNTSTASTPVAFTTSGTGGGASCDVAYVIESQWPGGFSAKVTVKNTGTTTVNSWTLTYPFPATGQAVSSGHSANWSQSGATVTATNLSWNGVLSPNASTTIGYNGLWTTTNPEPTAFKLNGATCTVS

SEQ ID NO: 58 - protein, 834 aa, Nonomuraea polychrome
MRKAAFIAAVSLALPLVVAGPAAPASAAPAFAYGEALQKSLWFYEAQQSGALPDWNRVSWRGDSGMNDGKDVGVDLTGGWYDAGDHVKFGFPMAFSATMLAWGAVEYRDAYASSGQLTHLLNNLKFVNDYFIKAHPSPNVLYGQVGNGGTDHAWWGPAEAMQMNRPAYKIDASCGGSDLAGETAAAMAASSIVFRPTNPTYADTLVTHAKQLYSFADTVRKKYSDCITDAQGYYNSWSGFNDELVWGAIWLHRATNDASYLAKAESGYANLGTEPQSTTKSYKWTIAWDDKSYGAYVLLHKLTGKQQYLDDANRWLDWWTVGVNGSQVKYSPGGQAVLDRWGSLRYAANTSFAALVHSDSITDATRKARYHDFAVRQINYALGDNPRNSSYVIGFGNNPPKNPHHRTAHGSWTDQITNPEQTRHTLYGALVGGPPDPNDAYTDKRDDYVMNEVATDYNAGFTSALARLYKEYGGAPAANFPVQETPDGPEIFVEAGVNASGSNFTEIKAIVRNQSAWPARVLSDGSFRYYFTLDGSTTASQISVSSAYTQCKAPTLHSAGGSTYYVDIDCSGQVIAPAGQSQHRREVQFRISSAGTWDPANDWSYRNIPTTPGATPARTQNITLYSGATKIWGEPPGPEEEDDTPPSKPGKPAVSGITGSTAKLSWTASTDNVGVTGYDVYLGSTKVGTATSASFDLSGLTPSTSYTASVVARDAAGNTSAPSDGTPFTTTNEEPPPGGCTAAFKVTSSWGGGYQAEVTVKNTSTSAINGWTVAWTSQNQISQLWGGKHTQNGSSVSVKNEAWNGTLAPNATATFGFIAGGTASTPDPITCTPA

SEQ ID NO: 59 - protein, 870 aa, Thermobifida alba
MSVTAPPPSPGGRRGPARRLLTALGSAAALAAGFLTLPAAVGTAHAEPAFNYAEALQKSMFFYEAQRSGALPEDNRVSWRGDSGLTDGADVGLDLTGGWYDAGDHVKFGLPMAFTATMLAWGAIESPEGYTLSGQMPHLKSNLRWVNDYFIKAHPSPNVLYAQVGDGDADHKWWGPAEVMPMERPSYKVDPSCPGSDVAAETAAAMAASSIVFADDDPAYAATLVEHAEQLYAFADTYRGEYSDCVPAGSFYNSWSGYQDELVWGAYWLYKATGDASYLDKAEYEYDFLSTEQQTDIRSYRWTVAWDDKSYGAYVLLARETGEQKYLDDANRWLDYWTVGVNGQRVPYSPGGQAVLDTWGSLRYAANTAFAALVHAETVADDPVRSQRYHDFGVRQINYALGDNPRNSSYVVGFGANPPRNPHHRTAHGSWTDSIASPADNRHVLYGALVGGPGSPNDAYTDDRQDYVANEVATDYNAGFSSALAMLVAEYGGTPLADFPPEEEPDGPEMFVEAQINTPGTTFTEIKAMIRNRSAWPARMLDEGTFRYWFTLDEGVDPDGITVTSAYNQCAAPDGVHHVSGDLYYAEIDCTGEEIFPGGQSQHRREVQFRIAGGEGWDPSNDWSFQGIGDELAPAPNIVLYDAGEPVWGNAPEAGEDPGEEPGEDRTPPSAPADLAATDVTATGAVLTWRPSTDHSGSGLAGYDVYAVEGDRAVLVGSTTQTSYPLTGLRAATAYTYRVAARDNDGNVSEHSSVSFTTLAGSGGGSSCLVEYATNDWNGGFTGSLRITNTGTEPLSAWELAFAFPAGQRITQGWNAVWTQSGTDVTATSLPWNSSLPPGTTVEVGFNGSWSGSNPHPAEFTLNGESCALA

SEQ ID NO: 60 - protein, 933 aa, Microbulbifer thermotolerans
MKSTKYLMKPLCAIIAGATLAVDAIAAPNYGEALQKSIYFYEAQQSGPIPDWNRNEWRGDSGMTDGADNNVDLLGGWYDAGDHVKFGFPMASSATMLAWGVVENREAYEQTGQLQHMMNNLRFVADYFVKAHTAPNEFWGQVGNGGLDHGWWGSAEVMPMERPSYKIDANCPGSDLAGETAAALAAISMVFESTDAAYATNLLEHAKQLYSFADNYRGKYTDCITDAASYYNSWSGYNDELVWGAAWLYRATGEQSYLDKAVSYYDNLSTEPQQPIKSYRWTHAWDDKSYGSYVLMAKLTGDDQYRADAERWLDYWSTGYEGNRVTYTPGGLAHLDQWGAARYAANTAFIALIYSDYLQSADPGNSRAQDYYNFAVGQMEYLLGDNRMNMSYQIGYGSVYPTSPHHRTAHGSWNDSLQDPVENRHTLVGALVGGPDTSDNFVNDRGDYVLNEVATDYNAGFTGALARLWLDFGGNPIPDSQFPAPETRDVELFVDAKLNSSSARHTEISSYVHNRTAWPARLTDNLSLRYWIDISELVDAGYGPEDLVLSSGYNQGSGFTGPHPWGDPADNIYYVEVSFAGVDIYPGGQSASKKETQYRIGAPEGAPWDPENDPSWDGYNSTRKEAPLIALYDDGELVWGQEPGGGCGGDTGVNCAPSADSQNLSTPYETSLSVTLSGSDSDGSIAAYTVVNGPANGTLEGSGASRLYTPAAGFFGTDSFTFTVTDNDGATSQPATISIEVEAPEIPAVNITSPVDGARFAPGADVRVQYNLQNAEGVNLYLDGTLVATGSGSGTLTVQAPDEIGFYMLKLVATQDGEELSASDSVTFEVAEAPAGSLGCTVGQMDDWGAGFVLNNITVTNEGSEPVDGWTVLLDFGQEVTGNHWNASLSSTSGSQLSASNVSYNGSLQPGQSATFGMQGTPGNLGAVTCTAQ

SEQ ID NO: 61 - protein, 880 aa, Lentzea flava
MLNRRSRAITTAIVVLFAGLVVPRAEAAPAFNYAEALQKSIWFYDAQRSGKLPADNRVSWRADSALRDGSDVGLDLSGGFYDAGDHVKFGLPFAFSMSMLAWGAVENRAAYADSGQLKHLMANLRWGTDWIIKAHPSPNVVYGQVGAGNPDHAWWGSAEVMPMARPSYKVDASCPGSDLAGEYAAAMASASMVFKDTDPTYAATLLTHAKQLYTFADTYRGKYSSCITDAANFYNSWSGYQDELVWGAIWLHRATGDASYLTKAKAEYQKLSTEPQSSERSYKWTIAWDDKSYGAYVLMAKLTGEQTYITDANRWLDFWTTGYNGNRVRYSPGGQAVLDSWGSLRYAANTAFAALVYSDWLSTRDSARARTYHDFAVRQVNYALGDNPRNSSYLVGFGANPPRNPHHRTAHGSWADSIQEPVTSRHVLYGALVGGPSTNNDAYTDSRSDYVMNEVALDYNAGFTGALARLYREFGGTPLASFPVAETPDGPELTVQASVNQSAAQFTEIKAFVLNKSAWPARTFNGTLRYFFTLDGATTPGQISVTTNYNQCGTASAPTQYRGAVYYVEIPCSGIAPAGQSAYRKEVQFRITSTGTWDPANDWSYAGLGSGNTVAQTDRIVLRQDGKTVWGTEPGGGVDDNEAPTVPTGLTASNAGSSSVTLNWTAATDNVGVTGYEVMNGTSVVGSATTTSLLVTGLVPDTQYTFSVRARDAAGNVSAASAPVSVRTLPGVRSTLSVQHKGTSAARTNQIGATLQVTNNGTGPVSLTTTTVRYWFTGEPAAGYQVWCDWAQIGCAGIRAQVVKLATPRPGADAYLEVSFTTGSLAANAATGEIQLRVAKSDWSAFDQADDHSYRVSAALTDFDRVTAYSAGALAWGAEP

SEQ ID NO: 62 - protein, 969 aa, Kibdelosporangium banguiense
MVTTVLVALLAGLIGPLPSVASAAPAFNYGEALQKSIWFYDAQRSGKLPADNRVSWRGDSALRDGSDVGRDLSGGFYDAGDHVKFGLPFAFSMSMLAWGAVENRDAYAGSGQLSHLMANLRWGTDWIIKAHPSANVVYGQVGAGDPDHAWWGSAEVMAMARPSYKVDTSCPGSDLAGEYAAAMASASMVFKESDPAYAGTLLTHAKQLFTFADTYRGEYSDCITDAAKFYNSWSGYQDELVWGAIWLYRATGDAAYLTKAKAEYLKLSTEPQSPERSYKWTIVWDDKSYGAYVLMAKLTGEQTYISDANRWLDFWTTGYNGNRIRYSPGGQAVLDTWGSLRYAANTAFAALVYSDWLSTRDSARAKTYHDFGARQVNYALGDNPRNSSYLIGFGANPPRNPHHRTAHGSWADNIQEPAVSRHILYGALVGGPKSNDDAYTDSRSDYVMNEVALDYNAGFTSALARLYKEYGGTPLANFPVAETPDSTELTVQASVNQSNTQFTEIKAFVLNKSAWPARIFNGTLRYYFTLDDATTPSQLAINTNYNQCGTPSAPTQYSGSVYYVEIPCTGVSPAGQSAYRKEVQFRITSSGTWNPANDWSYAGLGTGNTVVQTDGIVLRQDGKVVWGNEPTGTIDREAPSVPTALTASAVSGSSVTLSWAASTDNVGVTGYDVLRGGTVIASTTGTTYQATGLTPNTSYSFAVRAKDGAGNVSAASSAINVRTADTPIDTEPPTAPTGLRASSIGSTSVSLSWTASTDNVGVSGYEVLNSTTVVGSPTTTSLQVTGLTPDTSYTFSVRAKDSSGNVSAAVPITVRTTPATASTLTVQHKGTGSASSNQIGATLQATNRGTTSVGLTTVTLRYWFTGDASSPSYQVWCDWAQMGCANIRAQVVKLGTARTGADAYLEVSFTSGSLAPGASTGEIQLRVAKTNWSTFNQADDHSYRTSNSLTDFDRVTAYSGGTLSWGVEP

SEQ ID NO: 63 - protein, 1065 aa, Piscirickettsia litoralis
MVNLIIAFWGAAEVMQMARPAYRINTENPGSDLAAETAAALASMYLVLKRAGGDYAVYAEQKELLRHAKELFDFANQYRGKYSDSIRDAAQFYRSWSGYEDELVWAALWLYKATGEAHYLTYAENNYNNLLDEDHNGIQGRTAWTHAWDDKSYGSYVLLATLTGKEQYQHHAENWLNRWACTQTTDCVRRTSGGLVWLDQWGSLRYSANTALIAFIYADYLAKNNIKEDLVTSYQRFAKRQINYMLGDNSRQSSYVVGFGNNPPKNPHHRTAHGTWLGGSPLSIPNPSRHTLYGALVGGPGSTDDMDYIDNRNDYIRNEVALDYNAGFSGALARMVMEFGGRPLDNFPENAVRDDEFALEAKVNAQGVNFIEISALLRNKTAWPARITDNLSFRYFINLKEEINAGLTPENIAITTAFNQGAQVSALQVFDKEKAIYFIEIDFSGEAIAPGTQESHRREVQFRLSIPAELGISMNYQNDWSFREVIDGELTQDGRHLTHYIPVYEGNIRVFGLAPDSAPVNQAPIIDEDKSITIDQPSVPTALAISAPIDPEGGEVEVVITKVPVGASVIANSTGQVVHSGQKIAVNELTQLTVSYLGSNLTNDEFSYQAVDSAGNISTQRIRLLVENEAPIFGPNKTIAIYNQQYDQEIALNLPQPTDSNHDALTITVSRLPEHGQLTALGMVIRQGQTLSIAELSALNISYPIGTPINLVETLELIAQDARGGSAILTVQLTTKEQDLQQCTVKTELNEWENGFVAKLTVYNNSNLVVNDWQLDFKTKNYQVSNIWNADIATQGGVSSVSGKPYNQAIQVNNFISFGFQGVGHFQVPQAYLLNGQPCQQIGLSYKNRAPLMGENKIIQAAINDIVPLHLTLPEDPEGGVLDLNLVSVSEGVTVFAAGKKVFSGQSLTLSEFSQLAVQFPANTLANSSANLSLSAKDKQGARTVQTIRLIAKASDDFLVNCEVDFKIKDQWQNGFVADITIKNIGSELINGWQLTWDYNSQLINIWNARGEMKAGRVTLKNELYNSQIPAQGTVSFGFQGQGAAEVPEGILLNEEPCRIQSSNS

SEQ ID NO: 64 - protein, 790 aa, Myceligenerans indicum
MHPRPPARRRAAVRATALTAAWALLTAGAAGVAPAASAAGDQDYAEVLQKSLFFYDAQRSGDLPDDFRVNWRGDSGMADGADVGLDLTGGWYDAGDHVKFGLPMAFTTTMLAWGAIESPGGYAAAGQQDELLDSLRWVNDYFIRAHPEPDVLYAQVGDGEADHKWWGPAEAMQMARPAYKVDASCPGSDVAGETAAAMAASSIVFADSDPGYAAELVTHAEQLYDFADTYRGEYSDCVPAANYYNSWSGYQDELVWGAYWLYEATGDASYLAKAEAEYDQLGTEPQTGTHSYRWTIAWDDKSFGTYALLAQATGEQRYIEDANRWLDYWTTGYDGERVTYSPGGQAYLDTWGSLRYAANTAFVALVYGDWLAQQGDTTRARTYHDFGERQIDYALGDNPRGSSYVIGYGDNPPQNPHHRTAHGSWLDSLTDPADTRHTLYGALVGGPGSPDDSYTDDRSDYVSNEVATDYNAGFTSAVAYLADEYGGTPLSDFPQPEQPDQDEFFVESNLNQPGTEFTEIKAMVRNRSAFPARVLDDVRVRYWFTLDAGADPATIQASTNYTECPDGAATVGHAGGDRYYVEIDCSGSPVYPGGQSQHRREIQFRVTADVWDPSNDWSQAGVGADPAKNDRITLYEGPELLWGSEPGSVGPEPEPTEEPTVEPTGEPTGDPTEEPSGQPTDPGPGTASCTASYTVVNQWDGGFQGEVSVTNTGTETVEGAAVTWEFTNGEAITQSWGGTAVQDGPTVTVQAPGWAPDLAPGATWTLGFIATGSSVPPAAVLLGDTPCDLG

SEQ ID NO: 65 - protein, 803 aa, Microbispora amethystogenes
MRRTFPALAALIISSAVVPAAVPSTAAAAPAYNYAEALQKSLFFYEAQQSGKLPSTNRVSWRGDSALNDGKDVGLDLTGGWYDAGDHVKFGLPMAFSTTMLAWGAVDYRDAYTSSGQISYLLNNLRFVNDYFIKAHPSANVLYGQVGNGGDDHKWWGPAEVLPMARPAYKIDASCGGSDLAGETAAAMAASSMVFRPTDAAYADKLLTHAKQLYTFADTVRGKYSDCITDAAGYYKSWSGYNDELVWGAIWLYRATGDSSYLAKAESAYSGLGTEPQSTTKSYKWTIAWDDKSYGCYALLAKLTGKQQYADDANRWLDWWTVGVNGQKVAYSPGGEAVLDQWGSLRYAANTAFVALDYSDWLTDATRKARYHDFAVRQINYALGDNPRKSSYMVGFGANPPRNPHHRTAHGSWSDNLNEPAQSRHILYGALVGGPTSANDAYTDSRSDYTMNEVATDYNAGFTSALVRLVKEYGGTPLAGFPGTETPDGPEIYLQASVNATGSTFTEIKALVVNHSAWPARALSDGSFRYYFTLDGDTTPGQITLSSAYTQCKQPTGPTQFSGKIYYVTIDCSGQVVAPQGQSQSRREVQFRIASSGTWDPSNDWSYKGVATTPGSTPVTVENITLYSGSNKIWGNAPGTEQSPSPSPSASPSDSPSPSPSPSVSPSPPRSPSPSPTPTCPPQNTGSCDPPLSPSPSATPTQQGKACTATYKITSSWQGGFQADVTVKSSGTAAITGWTVGWTLPSGQSISQLWSGTLSQSGSSVTVKNLSWNGSLAPGATTSFGFTGNGNGSVPSPVTCTPA

SEQ ID NO: 66 - protein, 1058 aa, Cellulomonas persica
MRHRATSPHPSGALRRRGRTRVAAGLSLTLAASALVVPLATSASAAPDFNYAEALQKSMFFYQAQASGDLPDNYPVSWRGDSGLQDGSDVGVDLTGGWYDAGDHVKFGFPMAFTTTMLAWGAVESPTGYTKAGQLDELKGNLRWVNDYFVKAHTAPNELYVQVGGGEADHKWWGPAEVMTMARPAYKITASCPGSDVAGETAAAMAASSLVFKADDPTYAAKLVTHAKQLYSFADTYRGAYSDCVKDAQAYYKSWSGYQDELVWGAYWLYKATGDATYLAKAEAEYDKLGTENQSTTKSFKWTVAWDNKQFATYALLAMETGKQKYVDDANRWLDYWTVGAEGQKIAYSPGGMAVLDSWGALRYAANTSFVALVYSDWTTDSTRKARYHDFGVKQINYALGDNPRKSSYVVGFGANPPTMPHHRTAHGSWLDSITTPAASRHVLYGALVGGPSAANDAYKDDRQDYVANEVATDYNAGFTSALAYLVDQYGGTALASFPTPEQPDGDQMFVEAQLNQPAGGTFTEIKAMIRNQSAFPASSLKNAKVRYWFRTDGFAASDVTLSSNYSECGAQSGKGVSAGGTLGYVELSCVGQNIHPGGQSQHRRELQFRLTGPSGWDATNDPSFAGLTTTGLAKAPAITLYDGSTLVYGKEPTGTTPGDTTAPTVPGTPVASSVTTSGATLTWAASSDDTGVVAYDVYRVQGTTSTLVASPTTVTTTLTGLSAGTAYTYNVKARDAAGNVSAASSSVTFTTTEIPADTTAPTVPGTPVASSVTSTGATLTWTASTDAGSGLAAYDVYRVQGTTSTLVASPTTTTTTLTGLTPATAYTYVVRARDAAGNVSAASSAVTFSTLEIPADTTAPTVPGTPTASGITTTGATLTWAASTDAGSGVAKYEVLRVSGSTQTLVASPTTTSVALTGLTADTSYSYVVRAVDAAGNTSAASSAVTFRTLGSTPTQTCTVVYTPNSWNTGFTASLKITNTGTSALTWALAFDLTAGQKVSNGWSATWTQTGTSVTATGLSWNQTLAPGASTEIGFQGTHSGSNPSPTSFTVNGSVCG

SEQ ID NO: 67 - protein, 1046 aa, Cellulomonas gelida
MRRRRGRTRVAAGLSLALAATAIVVPLATSASAAPAYNYAEALQKSMFFYQAQASGDLPAGYPVSWRGDSGLTDGSDVGVDLTGGWYDAGDHVKFGFPMAFTTTMLAWGAVASPDGYTKAGQLDELKGNLRWVNDYFVKAHTAPNELYVQVGDGEADHKWWGPAEVMAMARPAAKITASCPGSDVAGETAAAMAASSIVFKGDDPTYAAKLVSHAKQLYSFADTYRGAYSDCVTGASAYYKSWSGYQDELVWGAYWLYKATGDATYLAKAEAEYDKLGTENQSTTKSYKWTVAWDNKQFATYALLAIETGKQKYVDDANRWLDYWTVGYNGQKITYSPGGQAVLDSWGSLRYAANTSFVALVYSDWVKDATLKARYHDFGVRQINYALGDNPRKSSYVVGFGANPPTKPHHRTAHGSWLDSITSPAQSRHVLYGALVGGPGSANDAYTDDRQDYVANEVATDYNAGFSSALAYLVDEYGGTPLASFPTAEQPDGDQFFVEAQLNQPPGGTFTEIKAIVRNQSAFPARSLKNAKVRYWFRTDGFAASDVTLSSNYSECGAQSGKGVSAGGTLGYVELSCVGQNIYPGGQSQHRRELQFRLTGPSGWDATNDPSFVGLTSTGLAKAPAITLYDGSTLLFGTEPTGTTPGDTTAPSVPGTPVASSITTSGATLTWAASTDDTAVVAYDVYRVQGTTSTLVASPTTTSTTLTGLTAGTAYTYNVKARDAAGNTSAASSSVTFTTTAVPTDTTAPSVPGTPVASAVTSTSATLTWTASTDAGSGVAGYDVYRVQGTTSTLVASPTTNSTSFVGLTPGSSYTYNVKARDVAGNVSAASASVTFSTVEIPPDTTAPTVPGTPVASGITKSGATLTWAASTDSGSGLAKYEVLRVSGTTLTLVASPTTASTTLTGLTADTAYSYVVRAVDQAGNTSAASSAVSFRTLADTPTGTCSVVYTPNSWNTGFTASLKITNTGTSALTWALAFDLTAGQKLSNGWSATWAQTGTTVTATGQSWNQTLAPGASAEIGFQGTHSGSNPSPTVFTINGTACA

SEQ ID NO: 68 - protein, 804 aa, Cellulomonas avistercoris
MPRTSARRARSRAVWAGAAVVAVVAGAVTLPAQAAQAAPAYNYAEALQKSMFFYQAQRSGDLPADYPVSWRGDSGMDDGKDVGKDLTGGWYDAGDHVKFGLPMAFTATMLAWGAIESPAGYTQAGQADELRDNLRAVNDYFVKAHTAPNELYVQIGKGDDDHKWWGPAEVMTMARPSYKISASCPGSDAAAETAAAMASASIVFKSSDAAYSATLLTHAKQLYSFADTYRGKYSDCVTDAQSFYKSWSGYQDELVWGAYWLYKATGDATYLTKAESEYDKLSNENQTTTKSYKWTVAWDDKSYATYALLAMETGKQKYVDDANRWLDYWTVGVNGAKVTYSPGGMAVLDSWGALRYAANTSFVALVYSDWLTDTTRKARYHDFGVRQINYALGDNPRKASYVVGFGANPPKNPHHRTAHGSWLDSLKDPVETRHVLYGALVGGPGSANDAYTDDRGDYVANEVATDYNAGFTSALAYLAGEYGGTPLAGFPTAERPDQDQFFVEAKLNQPQSGAFTEVKAIIRNRSAFPARSLTNASIRYWFTLDEGVPASSLSVSTNYTECGSQPAAIKHASGSLYYAEIGCAGQNIHPGGQSQHRREIQFRVQGTLQWDATNDPSFTGLPATGDPVKTKGITLHEGSTLLWGTVPGTVDPTPTPTVTPTPTATPTPTVTPTPTATPTPTPTPTPTPTPTPTPTPTSGPATCAVTYSANSWSTGFTASVRLKNTGSTPLTWSLTFDLTAGQQLQQGWSATWAQTGTKVTATGAAWNTTLVPGASVDLGFNGSHTGQNPAPTSFAVNGTTCAAG

SEQ ID NO: 69 - protein, 1051 aa, Cellulomonas xylanilytica
MPRPRSRGRVAIACTTAFTVAAGLLVAPLAGAATAADPTYNYAEALQKSMFFYQAQRAGDLPADNPVSWRGDSALTDGADVGKDLTGGWYDAGDHVKFGLPMAFSSTMLAWGAIAAPQGYTKAGQLDELKGSLRWVNDYFVKAHTAPNELYVQIGDGEADHKWWGPAEVMTMARPSYKVSAACPGSDVAGETAASLASASIVFKTDDPAYSATLLSHAKQLYSFADTYRGKYSDCVPEAAAYYKSWSGYQDELVWGAYWLYKATGDATYLAKAEAEYTKLSVENQTTTRSYKWTVAWDDKTYAMYPLLAMETGKQQYVDDANRWLDYWTVGVNGQRIPYSPGGQAVLDSWGSLRYAANTSFVALVYSDWLTDPTRKARYHDFGVRQINYALGDNPRKSSYVVGFGANPPQNPHHRTAHGSWLDSLTQPEKTRHVLYGALVGGPSGPNDTYTDLRSDYTANEVATDYNAGFSSALARLTQEYGGTPLAGFPVAEQPDGDEIFVEAMLNQPAGGTFTEVKAMIRNQSAYPARALKNGTLRYWFTLDSGDSAANLTLSANYSECGPQTAKPVSAGGTLYYAELSCVGQNIHPGGQSQHRRELQFRVTGGSTWNAANDPSFAGLGTALAKTKAITLYDGGKLIWGTEPTGTVTDTTAPSVPGTPVAAGITSTGASLSWTASTDAGSGVAGYDVFRVQGSTSTLVASTTTASTVLSGLTPSTAYSYTIKAKDVAGNVSAASSAGTFTTADPPVEVVVPGRPGTPVASAITSSGATLTWSASTAGTYPVAGYEVHRVVGTTATLVASTTGTSTALTGLTPGTAYTYTVRAKDSSGNVSPSSVGVTFTTTAATDTTAPTVPGTPSASSVTQTGATLTWAASADTGGSGLAGYEVYRVQGGTSTLVASPTTASTMLTGLAANTAYTYAVRAKDGAGNTSAASPSVTFTTLPTTSTSSCAVTYSANSWNVGFTGSIKVTNTSTSPLTWSLAFSFPSGQKLTQGWSATWAQTGSAVTATGLGWNATLAPGASTDIGFNGSHTGTNTAPTAFTVNGATCTVG

SEQ ID NO: 70 - protein, 924 aa, Streptomonospora litoralis
MPHPPSPYASSPPTPPPRRRSAARRRVTAAAACAALAAGLLASPTATGTADAAPDFNYAEALQKSMFFYEAQRSGELPEDNRVSWRGDSALQDGSDVGLDLTGGWYDAGDHVKFGLPMAFTTTMLAWGAVENPGGYTSSGQMRYLKDNLRWVNDYFIKAHPEPNVLYAQVGDGDADHKWWGPAEVMPMERPAYKVDPSCPGTEVAGETAAAMAASSLVFAEDDPDYAATLVRHAEQLYDFADTHRGDYSDCVPAGSFYDSHSGYQDEIVWGAYWLYRATGDESYLDKAETEYDNLSTEPQTDTRSYRWTIAWDDKSYGVYALLAMETGKQRYIDDANRWLDYWTVGVNGDRVPTSPGGQAVLDTWGSLRYAANTAFVALAYSDWLDQGTRKQRYHDFGVRQIDYALGDNPRDSSYVVGFGADPPVNPHHRTAHGSWLDDITAPEDNRHVLYGALVGGPDSPDDAYTDDRQDYVANEVATDYNAGFSSALAALVADYGGTPLADFPVPEEPEGPEIYVEAALNQPGTTFTEIKAMIRNKSGYPARMLDEGTFRYWFTLDEGVDPAAIEVTSAYNQCADPGGARQAEGDLYYVEIDCTGQEIYPGGQSEWRREVQFRIAGGEHWDPSNDWSYQGVGEELARAENIVLYDAGERVWGTAPDGGGDDGDDGDGDGTPDTEAPSAPGGLTADAATAGSVSLSWQPASDDTGVAEYTVHASADGSGEPVATVDGAQATVTGLEPDTEYTFTVRARDAAGNTSPSSAPVTAATSAEPEAGPLAALYRASGSDPSDSQIRAHLAVQNTGDAPVDLGRVSLRYWFTRDGGSAAELESHCDWAQLGCGGIARTIGDAPAVPGADTYLELSFDGGTLEPGGSTGPIQIRLNRSNWQAFDESDDHSYDGSAASFTENPNVTVYVDGEPVWGAEPAA

SEQ ID NO: 71 - protein, 920 aa, Nonomuraea spiralis
MIAAASLVLPLLAGVGPAAHAAAPAFAYGEALQKSLWFYEAQQSGDLPGWNRVSWRGDSGMNDGKDAGLDLTGGWYDAGDHVKFGFPMAFSATMLAWGGVEYRDAYAGSGQLTHLLNNLKFVNDYFIKAHPSANVLYGQVGNGGTDHAWWGPAEAMQMNRPAYKIDASCGGSDLAGETAAAMAASSIVFRPTNPTYADTLVTHAKQLYSFADTVRKKYSDCVSDAQGYYNSWSGYNDELVWGAIWLYRATNDAAYLAKAEAGYDNLGNEPQTTTKSYKWTIAWDDKSYGAYVLLHKLTGKQKYLDDANRWLDWWTVGVNGSKVKYSPGGEAVLDRWGSLRYAANTSFVALVHSDSITDAERKARYHDFAVRQIDYALGDNPRNSSYLIGFGANPPKNPHHRTAHGSWTDQITNPVNSRHTLYGALVGGPPDPDDVYTDKRDDYVMNEVATDYNAGFTSALARLYKEYGGAPAASFPPAETPDGPEIFVEAGVNASGTNFTEIKAIVRNQSAWPARVLSDGSFRYYFTLDGSTTAAQISVSSAYTQCKAPTLAPAGGSVYYVNIDCSGQAIAPAGQSQHRREVQFRISSAGSWDPSDDWSYKGIPATPGATPVLTKNITLYSGATKIWGEPPGPEEQDTTAPSKPGKPAVSGITGTAAKLSWTASTDNVGVTGYDVYLGSTKAGTATGTSFDLSGLTPATAYAAHVVARDAAGNTSAASDATTFTTTGTEDRTLPSKPGKPSVTGITGSGAKVSWAASTDDVGVTGYDVYLGSTKVGTATGTSYDLGGLSSATAYTVSVVARDAAGNSSAASDGTSFTTADAPAGGCAATFKVTNAWGGAYQAEVTVKNTGAAPVTGWTAAWSSAGQITQLWGGKLTQTGSAVSVRNESWNGGLAAGATTTFGFIANGTPATPDPITCTPA

SEQ ID NO: 72 - protein, 895 aa, Albitalea terrae
MTIRHGSRLRTPTSRGPRWTSWLTALATSAALLACGGGDPASTTTGRADASHTAPASAAATPAHNYAEALQKSVLFYEAQQSGKLPSWNRVSWRGDSRLSDGADVGHDLTGGWYDAGDHVKFGFPMAFTATQLAWGALRFEAGYKAAGQYDALLNNLRFVTDYFMRAHTAPNELYVQVGQGSTDHSWWGPVEVYPLAAPSFKISASCGGSDVAAETAASLASASMVFKASDAAYATQLLTHAKQLYTFADTVRAKYSDCVTDAANYYKSWSGYNDELAWAAAWLHRATGDSAYLAKAESFVNTSSSGFGTEGQSSFLPYKWTHDWDSKHYGVYVLLAETTGKAVYKDAIERNLDYWTTGTSTGERVSYTPGGLAWLSQWGSLRYAMNEAFIALAYADVVSDEAKKQRYRDFAVRQVNYVLGDNPRRGSYMVGFGSGSPQHPHHRTSHGSWMDSQTTPAQHRHVLYGAMVGGPGSDDSYADEIGNYVTNEVATDYNASLTGVLAKMNELFPGNVPLANFPQREQPSEDEIFVEAGINSSGSNYTEIKALLNNRSGWPARHGDKLSLRYYVDLGEVIAAGHSASDVTLSSNYSQGGTVSGLVRCGTSNLYYAVLDFTGTDIYPGGQSAFRHEIQFRFSVPAGTSYWNPANDPSYAGLSAAGTVKSLAIPVYSNGERVFGQEPAACGGVSVGVPTAPEGVTATGGSGSIALTWGAVANATGYRILRSATGAAGTYAQVGVAASPRFTDSGLAAATTYHYVVIATNAQGDGPPSADVSATTSTGGGGNGDCTLTLDASNDWGAGQVLVVKLANSGTQPLSNWQISWTESSDFTVQNSWSGSVSTSGRTVTVKPASWNSSIAPAGAIELGMQISYSGSRPLPTNAAVQGRSCTVSVISSR

SEQ ID NO: 73 - protein, 983 aa, Roseateles terrae
MSTLIQTRRGRTRWAAPCLALTLAACGGGDQTRSTASADAGPSAKALATSSGVKAAAAPAYNYAEALQKSILFYEAQQSGKKPEWNRVSWRGDSRLTDGSDVGLDLSGGWYDAGDHVKFGFPAASTATQLAWGALEFEAGHKDAGQYDALLRNLRWINDYFIKAHPSANVLYGQVGNGSTDHSWWGPVEVYPKEAPAYKIDATCGGSDLAGETAAAMAAASMVFKTSDPSYSATLLTHARQLYDLADTKRQKYVECITDAQGYYNSWSGYFDELAWGAIWLYRATGEASYLTKAESFVTTGAGGFGSEGQTAYLPYKWTQDWDSKHYGSYLLLARLTGKQKYKDAIERNLAYWTTGTSEGERVTYTPGGLAWLSQWGSLRYALNESFIAMVYAKDVSDSTKQQRYRDFAMQQLNYALGSNPRNSSYLIGFGANSPQHPHHRTAHGSWADSQTLPANHRHVLYGALVGGPGANDAYTDSISDYVSNEVAIDYNASIVGVLAGAQQLFPGSVPLANFPQAEVPTDDEYFVEAALNSAGTTYTEVKAVVNNRSGWPARVTDKLSLRYYVDLSEVFAAGRTAADIKVSANYSDGGVARGLVRCGSSNIFYAVGDFTGTKVYPGGQSAYKKEIQIRLAAPDSTTFWNPSNDPSYAGLGTTGSAPLKVSGITLYDNGRKVWGNEPTACGGVAVPVPATPSGLTATAGIGSISLSWAATANATGYTLKRSTTGTAGSFALLASPTGTSYVDSGLAGSTRYYYTVSARNDSGESAVSDAVSATTPAGPPAAPAPTAAAGDGKVTLSWSAVTGAASYEVSRASSATGSYTSLASGLSALSYVDSGLTNGSAYFYKVGARNSAGVTVSDPVSATPVGSGGGGGGSSNCTLTLDTSNDWGSGQVFRLLLSNTGTTPISNWGVSFTESNSITVSNSWSGTFVATGTKVSFTPQTWNATVAAGGNTDAGMQLSYSGTKPTPSAVVMTGASCQVVIK

SEQ ID NO: 74 - protein, 946 aa, Candidatus Lokiarchaeota archaeon
MKDFRILFTKKVKFLLILFIFLFQSTLFLGNSINKNMINNHELKINNWEEIPITDTNGEALYNYATMVSRTILFYDANQCGNNITGNNRFNWRGDCHLNDGIEWTLNNGETIWIDATGGYHDAGDHIKFGITNAYAASTLGWGLYEFRDQYEETGQLDDILRVIKWATDYFIKCHPEPNSFLYSVGSGEDHNYWGPPELQLDSRSPRDIIVCTPSNPASDVCGLTAGALALMYINYYDIDEEYANLCLKHAKEIFELGRNNLGLSDGLAFYPPSIYWDDLAWGALWLYAASNDISYYNEFLTILGPSCAGIDHDDDIENGLNWENQWTHCWDAVWAGVFAAAAGITAHPAFLEQVKINLDFWLYDLDETPAGLKYLNEWGVLRYSTASALCGLVYYKYTNDIVYRDYAASQINYALGSNPLQRCYIVGCGNNPPQHPHHDAAHGSETGFLDDPLEHKHILYGALVGGPGMDDQHNDATDDYVQNEVSIDYNAAMVGALAGMRKYFGPNLIPDPDPLPEEPIQAYWVESKIEKDQNERSQLTFYVQNHAIHPPHYENNLIFRYFVDLSEVFEQDYGLENVNIEVVVNENQEAQVSQELIPWDELNYIYYVEISYQGVELYNKREVQIALIYNTPNFEGIWDPTNDFSHLPMTDQLESNENIPLYRGEELIWGLEPYKDRIPPEPPSGIITTVKGSAQIDLDWNDNTEEDLSYYNIYHSTAVNFECNMDSFIGSSKTSEYSDTELLQETTYNYKITAVDSNQNEGMPSEIVSATTLTPDPYPPDPPTGLSIISTSSSQIELDWNDNTESDLSKYKIYRSTSMGFICDQVNFIGDTLVSIYLDSNLLANTIYYYKIIAIDTSNNPSNPSFEIIAQTDKILAGLKVQYRSGNLNQLSSEIRYELILFNNGPLDVALTDVTMRYWFTSEPSLSDLTYSCDYAAVGASGIST

SEQ ID NO: 75 - protein, 885 aa, Halomicrobium salinisoli
MTRETRDRSQTGESETNGGGSPHRTERRTFLKAGLVGLGTVAAGGPGAGVASAVAKDDYNLAHALQQSLYFFDANECGDGVTGQPLEWRGDCHLVDESVPLRQSIDEGGSNVPESYVDEHSDVLDPNGDGTVDLSGGFHDAGDHVKFGLPQAYAASTLGWALYEFEDAFREIGSDDHIRRHLRRFTDYFLKSIFRDDDGTVVAFSYQVANGNFDHEYWGPPELQDNEEMPRPAYLATPETPASDQCGAAAAALALQSLNLEDEDPEYAERCLDNAQALYEFGVEHRGLGHGGEFYPSSSDADDLAWAAVWLHVATGEQRYLEDVIGTDGSGNYTGHVGEIIATTEDEWNNTWVHSWDTVWSGVILKLAGITDDQQWWDLARWHLEYWTGGEVSHDGSSGNYIQTTPAGFSYLDQWGSARYNASAQFLATVYRKQGGEKGEALTDWARGQMQYIMGDNPFGYSLIVGFTEDHAEEPHHDAAHGSLVGQPSDPPKPKHTLWGALVGGPDDQDEHNDETDDYIYNEVAIDYNAGFVGALAGLYTYYGDGDEPVEGFPPDEEPIDPYYVEAQVEGNSAETVEIKAFINNVAVHPPRFEDGLSFRYFFDASGIDDPEGLTVDVFYDQVGSQLGQPAEVVGPEPYDESEGVYYVEFDYSGTAIAGRYELTFALSDYTNASLDASTDWSFQDLGDGLTETEYMPVYVDGELAFGREPGGNAPDTTAPGAPSPVSVTSTGTTSLGLSWSAVTDDGSGLDQYVVAVDGTERTTVPAGTTSATVDDLDPDTDYEISVRAVDGAGNESEPATATATTDTEGGGDGPEPLDGTRPTDPDGDGVYEDLNGNGETDYSDVVRYFDEIDGEYFAENAEYFDFNGNGEADYSDLVALFQEV

SEQ ID NO: 76 - DNA, 3135 bp, Cellulomonas palmilytica
  source 1..3135: mol_type = genomic DNA
gtggtttcctggtcccgccgccgtgcacgcacggctgtcctgtctgccggcgtcaccgccgcagccctgatcaccgcgacggtcgtcgcgcccgccgccctcgccgaccccgtggagtcgtcgcacgacttcgagtccgggacgcagggctggtacagctacgggggcgactcgtcgtcctcggtcgtcgacggcgagctctgcgtggtcgtgcccgccggcacgacgaacccgtgggacgtcgcggcgcagcacgacgacgtgacgttcgaggcgggcggcacctacacggtgtcgttcaccgcgcacgcgaccgaggaggtctcggtcaacctgcgggcgggcatcgggtggccggacgacgtgtcgtcgaccgtcgccctggcacccgaggagcaggagtactcgttctcctggacccccgagttcagcgggtccggcaacctgtcgttccagctcggggcgcaggacgtcgactacacgttctgcctcgacgacttccagctggtcggcgaccgggagctgatcccggacaccagcttcgacggtgtgctgcccgagggctggagcgcggccggctggaccgtgacgtccgagccgggtgagggcgagccgctgtgcttcgaggtcccggccgactcgaaccagtacgcgggtctcgtgctcaacggcctgccgatcgaggagggctccaactaccgcctcacgttcacggccagctcgagcaacgccgcgacgatccgcacgatcgtcggcgagaacgccgagccgtggcgcgcggccttcgccgacaacacgccgctgtcgaccgagctgaccgagcacgtgctcgcgttcacctcgcccgtgtctttcccggccgagtccgacgacccggcggtcggcgtcgggcaggtcgcgctgcagatgggtgaccgcggcgcgttcacgttctgcatcacgagcctgtcgctcaagaaggtcgccacgccgccgccgccgtacgagcccgacgtgcgcggtcccgtgcgcgtcaaccagctcggctacctgccggacgggccgaagaacgcgacggtcgtgagcgacgcggccgagccgctcgcgtggcggctcgaggactcgtcgggcaccacggtcgcttccggtgaggcgacgcccgccgggctcgacgcctcgtcgcagctcgaggtgcagacgatcgacttctcgtcgtacaagaagtccggcaccggctacacgctcgtcgtgggggaggaccgcagcgacccgttcgcgatcgcgtcggacctgtacgcccagctgcgctacgacgcgctgaactacttctacccggtgcgctcgggcatcgcggtcgacgtgccggacgaccggtacgaccggcccgcgggccacgtgtccgggccggacggcgaggtcaacaagggcgatctcgacgtgaagtgcctcaccgcggccgaggacggcgcgagctggtcctacggcacgtggacctgcccgacgggctactcgctcgacgtcgtcggcggctggtacgacgccggtgaccacggcaagtacgtcgtcaacggcggcatcgcggtcgcgcagctcctgagcacgtacgagcggaccctgcacacgaagaacgcggcgaagggcgccctggcggacggcacgctcgacgtgccggccgacgagagcggcaacggcgtgccggacgtcctcgacgaggcgcgctgggagctcgagttcttcctcgcgatgcaggtgcccgcgaagtccggcatgacggtctccgcgaccgaccagcgctcgctcgacgggctcgtgcaccacaagatccacgacgtcggctggaccggcctggggctcctgccctcggaggacccgcagcagcgacgcctgcaccggccctcgacggctgcgacgctcaacgtggccgccacggccgcgcagggtgcgcgcctgttcgagaagtacgacaaggcgttcgcccagcagctcctgagcgtcgcgcggtccacgtacgcggccgccaagcgggtgcccgacctgtacgcgccgcagtccgcgggccagaacggcggcggaccgtacgacgacacgaacgtgaccgacgagttctactgggcggccgccgagctctacctcacgacgggcgagaaggccttcgagaaggacctgcagaacagccccgtcgcgacgagcgacatctgggagcgttccgggttctcgtgggggagcgtggcggcgctcggtcgcctcgacctcgcgacggtgccgagcaagctggcgacccgcaaggccgtgcaggcgtccgtcgtggccggggccgacaagctcctcgcgtggcagcgggccgaggcgttcggcaccgcgtacccgggcaacgacgacggctcgtacgtctggggctcgagctcgtccgtgctcaacaaccaggtcgtcctcgccacggcgtacgacctgacgggcgacgcgaagtacgcgaacgcggtgctggagtcgatggactacctgctgggtcgcaacgcgctgaacaactcgtacatcacggggtacggcacgaagttctcatcgcaccagcacagccggtggctcgtcccgccgccgccggggacgatctcgggtggcccgaactcgcagagcagcacgtgggacccggtcatgaaccggatctacccgcccggtcacgactgcgccccgcagctctgctacatcgacgacatcgaggcgtggtccgtcaacgagctgaccatcaactggaacgccccgatgtcctgggtcgcctccttcgtggccgacctcggcgtgagcggggtcccggccgcgccggtcgtgaccaagcacccgtcgtcggtgtcggcgaagaagggcacctcggtcgcgctgagcgctgctgcggccggcaagcctgcgccgaccgtgcagtggcaggtgcgcaacgcgaagggcgcatgggtcgacgtgcccggcgggaagacgacgacgctgcgggtcatcgtgtcggcggcgaccgacggcgcccggtaccgcgcggtgttccgcaacgcgtccggcacggtcgtcacggacgtcgcgacggtcacggtgctgcccggccccaagggccccgcgccgaagggcccgggtccgatcgtccccgcacccaagccgggtcccggccgcgggtga

SEQ ID NO: 77 - DNA, 3639 bp, Cellulomonas gilvus
  source 1..3639: mol_type = genomic DNA
gtggtttcctcgaagtacccctggcgtgcgccccggtccgggcacgcgcgcactgcttcgctcgtcggtggcgtggccgccggcgctctcgtgatcggcgtcgccgtcgcccccctggcggccgccgaccccctcgacgaggtgcatgacttctccgacggcacccagggctggtacggctacgggggaggacccgccatgtcgaccggcgtcgtcgacggcgagctgtgcgtggtcgtccccgccgggaccgacgagccgtgggacgtcgcgatccagcacgacgacatcgacttcgccgcgggtgaccggtacacggtcgggttcaccgcgcacgcgacgtccccggtcacggtcaacctgcgcggtggcatcggctaccccgacgacgtggcctcgtcggtctcggtcggcaccgagaccgacgcctactccttcacgtgggagcccgagttctccgggagcgggaacatctcgttccagctcggcgcgcaggcccaggactacacgctctgcatcgacgacttcgtgatcgacagcggccaggagctcgtgcccgacacgacgttcgacggcgtgctgcccgacggctggaccaacgacggctggacggtcacgtcggaggccggcgagggcgagccactgtgcttcgaggtccccgggaccgccggcacgtacgcggggctggtgctcaacggcctgccgatcgaggagggcggcaactacgagctcacgtacaccgcgagcgcgtccaacggcgcgacgatccgcacggtcgtcggcgagaacgcggcaccgtggcggaccgcgttcgtcgacaacaccgagctcagcacggacctgaccgagcacgcgctcgcgttcacgtccacgttcacgttccccgcggagtcggccgaccccgcggtcggcgtgggacaggtcgcgctccagatgggcgcgcgcggcgacttcacgttctgcatcacgagcctgtcgctcaagaaggtcgcgaccccgccgccgccgtacgagcccgacacgcgcggtccggtgcgcgtcaaccagctcggctacctcacgaacgggcccaagaacgcgacggtcgtgagcgagtcggccacgccgctcgcgtggcagctgcaggacgcctcgcacgccgtcgtcgccgagggtgaggcgacgcccgcgggggtggacccgacgtcgcagctcacggtgcagacgatcgacttcagcgacgtcaccgcggcgggccagggttacaccctggtggtcggcgaggaccgcagcgacccgttcgcgatcggcgacgacctgtacgagcagttccgctacgacgcgctcaactactactacccggtgcgctcgggcatcgcggtggacgtccccgacgaccgctacgaccggcccgcgggccacgtggacgggcccgacggtgccgtcaacaagggcgaccaggacgtcgcgtgccttacggcggccgacgacggtgcgtcgtggtcctacgggtcgtggacgtgcccgcaggggtactcgctcgacgtcgtcggtggctggtacgacgcgggcgaccacggcaagtacgtggtgaacggcggcatctcggtcgcgcagctcatgagcgcgtacgagcgcacgctgcacgtggaccacgcgagcgacgacgcgttcgcggacggcacgctcgacgtgccggccgacgagagcggcaacggcgtgccggacgtcctggacgaggcgcgctgggagctcgagttcttcctggcgatgcaggtgcctgcgggctccggcatgacggtctcggacaccgacgaccggtcgctggacggcctggtgcaccacaagatccacgacgtgggctggacgggcctggacctgctgccgtccgccgacccgcagcagcgccgcctgcaccgcccgtcgaccgcggccacgctcaacctggcggccacggccgcgcagggcgcgcgcctgttccgtgcgtacgacgccgcgttcgcggacgagctgctcgaggccgccgagaccgcgtacgcggccgcgcagcgcgtgcccgacctgtacgcaccggcctcggccggcgccaacggcggtggcccgtacgacgacgccgacgtgtccgacgagttctactgggccgcggccgagctgtacctgaccacggccgccgacgagtacgaggcggacgtgctggcgagcgagcacgccgacgacgacatctggacgcgcggtgcgttcagctggggcgcggtcgccgcgctcggccggatggacctggccacggtgccgagcgagctgccgaccagggccgcggtgcgtgcctcggtggtcgagggcgcgcagaagtacctcgcgtggcagcaggccgaggccttcggcacggcctacccgggcggcgaggacctctcctacgagtggggctccaactcgatggtgctcaacacgcaggtgatcctggcgaccgcgtacgacctgacgggtgagccggcgttcgcggcggccgtcgtcgagtcgatggactacctgctcgggcgcaacgcgctcaacaactcctacgtgacgggctacggcacgacgttctcgagccaccagcacagccggtggctcgtgccgccgatgccgggcacggtcgcaggtggaccgaactccaagcgcggcacgtgggatcctgtgatgaacgggctctaccccgagggccacgagtgcgccccgcagctgtgctacgtcgacagcatcgaggcgtggtcggtcaacgagctcacgatcaactggaacgcgccgctggcgtgggtcgtctcgttcgtcgacgacctgggtgcgggcgtcacggcccaggcgcccgtggtgaccacgcagcccgcgtccgtgacggtcgcgctgggggccaaggccacgttcacggccgcggcgagcgggacccccgcacccacggtgacgtggcagtggcgggcgcccggcggcacgtggaagacggtcgcgggtgcgacgagcccgtcgctgacggtcacggcgaccgccgccaccgacgggcgtcagtaccgcgcggtgttcaccagctcggccggcaccgccacgagcgacgtcgccacgctgcgcgtccgtgcggtcaagcccgtggtcaccacgcacccggcgtcggcatccgcggcgctggggcgcaaggtcacgctcagggccgacgcgtccggctacccgacgccgacggtgcgctggcagcagcagcggcccggctcgtcgacgtggacggacgtgaagggcgcgacctcgcgcacgctcgtcgtcgcggtcacgcgggcgaccgacggcgtccggtaccgcgcggtgttcaccaaccgcgcgggctcggcgacgtcgcgtgccgcgaaggtcacgctggtgcgcgccgctccgcggttcgtcgaccaccccgacggcacgaccgtgcgtgccggtcagaaggtgacgctctcggcgaccgtgctggcctacccggccgcgacgctcacgtggtacgtcaaggcgcccgggagctcgagctggaaggccgtgcccggtgcgacgggcacgcggctcacgctcgtcgcgagccggtcgctcgacggcgcgcagtacaaggtggtcgcgcgcaacgcgctcgggtccgcgtggtcgaaggccgcgctgctccgggtccgctga

SEQ ID NO: 78 - protein, 1054 aa, Cellulomonas sp.
MAAPCGHLARLRGDDVVSWSSRRARTAVLSAGLTAAALVAATVVAPAALADPVGSTHDFESGAQGWHSYGGASTSSVVDGEFCVVVPTGTKDPWTVAAQHDGITFEAGTTYTVSFTAHATQDVSVNLRAGIGWPDDVASTVALTSQEQEYSFTWNPEFSGEGNLSFQLGAQAADYTFCLDDFELAGDAELIPDTSFDGVLPEGWSAAGWTVTSEPGEGEPLCFEVPAGSGQYDGLVLNGLPIEEDANYRLTFTASASNAATIRTIVGENAVPWRAAFADNTQLSTELTEHELAFTSPVTFPAQSDDPAVGVGQVALQMGARGDFTFCITSLSLKKVATPPPPYEPDVRGPVRVNQLGYLPDGPKNATVVSDATEPLEWRLEDSSGATVASGDATPAGLDPSSQLTVQTIDFSSFRTAGTGYTLVVGEDRSDPFAIAKDLYAQLRYDALNYFYPVRSGIAVDVPDDRYDRPAGHVSGPDGEVNKGDLDVACLTAAEDGAAWSYGTWTCPTGYSLDVVGGWYDAGDHGKYVVNGGIAVAQLLSMYERSLHSPDAARGALKDGTLDVPADESGNGVPDVLDEARWELEFFLQMQVPTGSGMTVSATDKRSLDGLVHHKIHDVGWTGLGLLPSQDPQERRLHRPSTAATLNVAATAAQGARLFAKYDKAFSQQLLSVARSTYAAAKRVPDLYAPQSAGQNGGGPYDDTDVSDEFYWAAAELYLTTGEKTFETAVQSSPLATSDIWARSGFSWGSVAALGRMDLATVPSKLPTRKAVQASVVTGAEKILAWQQAEAFGTTYPGDADGSYVWGSSSSVLNNQVVLATAYDLTGQARFANAVLESMDYLLGRNALNISYITGYGTKFSSHQHSRWLVPPPPGTIAGGPNSQSSTWDPVMNRLYPPGHDCAPQLCYVDDIEAWSVNELTINWNAPMSWVASFVADLGVSGVPTAPVVTKHPSDVSAKKGTSVALRATASGKPAPSVQWQVRDAKGRWVDVPGARTTTLRIVVTPATDGAQYRAVFRNASGTATTDVARVTVLAGPKGPGPKGPGPIPWPGRG

SEQ ID NO: 79 - protein, 1044 aa, Cellulomonas palmilytica
MVSWSRRRARTAVLSAGVTAAALITATVVAPAALADPVESSHDFESGTQGWYSYGGDSSSSVVDGELCVVVPAGTTNPWDVAAQHDDVTFEAGGTYTVSFTAHATEEVSVNLRAGIGWPDDVSSTVALAPEEQEYSFSWTPEFSGSGNLSFQLGAQDVDYTFCLDDFQLVGDRELIPDTSFDGVLPEGWSAAGWTVTSEPGEGEPLCFEVPADSNQYAGLVLNGLPIEEGSNYRLTFTASSSNAATIRTIVGENAEPWRAAFADNTPLSTELTEHVLAFTSPVSFPAESDDPAVGVGQVALQMGDRGAFTFCITSLSLKKVATPPPPYEPDVRGPVRVNQLGYLPDGPKNATVVSDAAEPLAWRLEDSSGTTVASGEATPAGLDASSQLEVQTIDFSSYKKSGTGYTLVVGEDRSDPFAIASDLYAQLRYDALNYFYPVRSGIAVDVPDDRYDRPAGHVSGPDGEVNKGDLDVKCLTAAEDGASWSYGTWTCPTGYSLDVVGGWYDAGDHGKYVVNGGIAVAQLLSTYERTLHTKNAAKGALADGTLDVPADESGNGVPDVLDEARWELEFFLAMQVPAKSGMTVSATDQRSLDGLVHHKIHDVGWTGLGLLPSEDPQQRRLHRPSTAATLNVAATAAQGARLFEKYDKAFAQQLLSVARSTYAAAKRVPDLYAPQSAGQNGGGPYDDTNVTDEFYWAAAELYLTTGEKAFEKDLQNSPVATSDIWERSGFSWGSVAALGRLDLATVPSKLATRKAVQASVVAGADKLLAWQRAEAFGTAYPGNDDGSYVWGSSSSVLNNQVVLATAYDLTGDAKYANAVLESMDYLLGRNALNNSYITGYGTKFSSHQHSRWLVPPPPGTISGGPNSQSSTWDPVMNRIYPPGHDCAPQLCYIDDIEAWSVNELTINWNAPMSWVASFVADLGVSGVPAAPVVTKHPSSVSAKKGTSVALSAAAAGKPAPTVQWQVRNAKGAWVDVPGGKTTTLRVIVSAATDGARYRAVFRNASGTVVTDVATVTVLPGPKGPAPKGPGPIVPAPKPGPGRG

SEQ ID NO: 80 - protein, 1152 aa, Sanguibacter inulinus
MVSPRHARQALAVLTASSISAALVLATVVAPAATASPLSTEHTFTGGDPEGWHSYANSGSVTTSTATEELCSTVSGGTNPWDVAAQHDDLTFDRDTTYTMSFDARADQAVTLPLQVGATYPATSARQVVLDGTATPQHVELTVSPATWPTTSTDAGSPLADGWTTTSGLAVLQLGAQAVPYTVCIDDVSITERTDPTDPTDPTDPTDPTDPTDPTDPAEQVVGGDFEDGAIAPFYAVGPGVTSAVTDGVLCVELGAGTSNRWDQIVGYNGIVIEPGTTYTLSFDASSSNGRPVRAIIGDDEDPYTVLLENNPTLSAGMRSYTYTFTAERGFPTDGATSPGIGEISFQVGGSDTPWTFCLDDVSLLSGTETPPYTPETGPRVRVNQVGYLPAGPKEATLVTDAPAALPWELHDAQGVTVASGTTVPAGLDPTAGVAVHEIDFTGVTAPGTYTLVADGEESYAFTVGADIYQQLRADAINYFYPARSGIEIDGSLMVGQPDAQKYSRPAGHVGTPDGADGGPNRGDVDVACLTPETEGQYWMYGDWTCDYTADVTGGWYDAGDHGKYVVNGGIAVAQLLSTYERTLTSPTGAGADLRDGTLNIPLDEQTNGVPDVLDEARWELEWMMKMQVPADAAMYPGMVHHKVADADWTGLPLLPSQDAQERYLHRPSTAATLNFAAVAAQGARLWEQYDPQFAADLLAAGRVAWDAALRTPDLYAPAPNDDPSTGSGPYDDDSVGDELYWAAAELFLTTGDQVFEDHVLASEYHTADIWTSGGFNWFETAALGRLDLATVESDLPGRAAVRDSVVEAAERYLAWQQGEPFGTAYPGSDGIYSWGSNSAILNNQVVLGTAFDLTSDQRFADAVLESMDYLLGRNALNNSYVTGYGQQFSQNQHSRWFAHSLEPSLPNPPRGSVSGGPNSDAVTWDPVIKALYGPDRMCAPQACYVDDIQSWSTNEITVNWNSALTWVASFVADQQDGDASGAGTVVVVTSDPADVTVVDGQQASMSAAATGSPAPGVQWQRLTAGVWTDVPEATSTTLTLTARLGEGTQYRAYFTNAFGGVYTEPATLEVTESPEVVAPSPAPVVDPPVEETARGAGGGAPDGRGGALATTGADPAWVVVAGLLLVAGFTVTGVARRGRRSRDGSAPGARA

SEQ ID NO: 81 - protein, 1089 aa, Cellulomonas xiejunii
MRAATAITSAGVLAAVGLLPAVAVDDGFDDGPGAWIAYGTDGGIDTSSGALCVDVPADSAQYGVGVVLNGVAVEEGATYTFAYTASATTDVTIRALVGQNGAPYGTVLDTTPALTSEATSVEETFTASASYPATPTGESPEGQIAFQLGGFTDEAWTFCLDDVALSSDSELLPHTSFAEGLGPWDLYGTSEPTFADGEMCVEVPGGNANPWDAGLSFTGLPIEEGQNYVLSLTGRTEPATPVRVIVGEGGGAYRMVLEQSAAPLGVEQTTLSYPFTAGLAFPADGEAPGQVAIHLGKSAAYTFCLSEVSLTTSATPPPPYEPDTGPRVRVNQVGYLPFGPKRATLVTEATDAVAWQLLDADGDEVATGESQPVGADASSGENVHVIDFSDVETAGAGLTLVADGETSHPFDIAEGLYSQLRYDALNYFYLARSGIDIEASIVGEEYAREAGHVGVAPNQGDTAVPCIGPRDYYDGWTCDYTLDVTGGWYDAGDHGKYVVNGGIAVAQLLSTYERTLTTAGARAGALDDGTLNLPEHGNGVPDVLDEARWELEWMLKMVAPSGEYAGMVHHKIHDEGWTGLPLAPADDPQARSLHRPSTAATLNVAAVAAQGARLFAEYDEQFAATLLETARSTYEAALAHPDVYAPGAAGSDGGGPYDDSDVTDEFYWAAAELYVTTGEDRYATDLTASAHHTGDVFGPGGFHWGGTAALGRLTLATVPNDLPDRADVRASVVAGADRYLAAQAEQPWGSVYSPTGGSYDWGSNSSVTNILVVVATAYDLTGDEKYLRGTLEGLDYLFGRNALNQSYVTGWGTVSSQNQHSRWFAAQLDPSLPNPPPGSLAGGPNSMRGTWDPTMQATLPEDCAPATCYLDEISSWASNEITINWNSSLSWVASFAADQEDGGVVAVAPAITTQPVDVTVALGGTATFTAAASGVPAPQVQWQVREGGKWRDVPGATAATLQVVATAAKAGTQYRAVFTNASGSATTDVARLLVEKQAPVVVKHPADVSGRIGTFVSFTAGASGNPTPTVRWQVRWGSGPWLPVPFAHSTTLRVLVTPLAYGTQYRAVFTNPAGTAATDGAHLTPRFGR

SEQ ID NO: 82 - protein, 1121 aa, Cellulomonas cellasea
MVSFPDASARASGAAAATVAVLALAAAVSPVALAGPLTDVHDFATGTEGWFSYPPEAVTASAETGELCAAVGAHTGNPWDVAIQHDAVTFDRDATYTVEFDAHASRAVTVPVQGGPGYPAVFGQSVQLDGSSTPEHVELTFSPSAWPTGEGSPVADDWSTATGNVSFQLGNQGEPYELCVDDFSIRPLGANLIPNGDFASGSTDPWFVAGDLGPADVSSGALVVPVPAGSGIYAGVGFNGLAIEEGTSYTLTFDVSASEERTIRAIVGENGGSYGTVLDKTVPVTTEPTTVSHTFTATDSYPATTTPGGPFEGQLAFQVGGRGGDWTFTLDNVSLVESLTPPPPYAPETGPRVRVNQVGYLPAGPKQATLVTERTEPVVWQVRTVSGGVLAEGVTTPRGVEPSSGLNVHTIDFGELATEGEGLILAADGETSRPFDIDADLYQQLRYDALNFFYLMRSGTEIDAAIDPDYARPAGHVDVAPNTGDGAVTCLTAADEGAGWAYGDWTCPEGYALDVRGGWYDAGDHGKYVVNGGIAAAQLLSTYERTLHAGTADPGALADGTLAVPESSNGVPDVLDEARWELEFLLSMQVPADGGEYAGMTHHKIHDVGWTGLPLLPSQDPQERRLARPSTAATLNLAAAAAQGARLFAEHDAEFAAELLAAARTAWDAAQAHPAVLAPDAAGNNGGGAYGDGTVTDEFYWAAAELFLTTGENAFAEHLLASPHATGDVFGPGGMNWGSTAGLGRLDLATVPNALPTRDAIRQSVLDAADAYVATQDAQAWGSTYVRPDGLYEWGSNSMVLNTTVVIATAFDLTGDETYRRSALEAVDYLLGRNALNISYVSGYGEVSAQHQHNRWFPLSVPGVSPEGSLAGGPNSTTGTWDPTMQAAFAGGCAPSMCYLDELGSWASNEVAINWNSALSWVASFVADQGDGSADASTPVRVTGQPADVTVAPGATATFTAAATGVPAPTVRWQVQRPGKRWVDVPGATSTTLTVTATLADHGTLYRAVFTNASGSETTDVARLAVVAAEPLTVRLSAPAVRAGGHVTVAVRGATAREIVQVRLGDELLETRAVRKDGTVSISVHVPRHTDPGAAVVTVRGSSSGREGTATLQVLPAPRRS

SEQ ID NO: 83 - protein, 958 aa, Amycolatopsis bullii
MRVRRFRAGLVLLAVTTLGLTAGSVPASAADYERLLNGTFASGTLDPWWAGAGTTGRVTGGEFCTDVTGGTANGYDALAGQNGVPFEAGQQYTLTFDAHATTTQQISAVAGEAVSPYRQISRTDLTVTPTAQHFTVTFTSTLDFPAAGNGQLAFWFGGQAANNTVCLDNISLIGGVIPPGGLPPTSGIRVNQESYTPGLPKHATLISSSATPVTWTLRNAAGTAVTTGQTRPRGADAASGESVHDIDFSAYDTAGTGYTLAVGSETSFPFDISADGLKKLRYDSLAFFYHQRSGIAIDAQYVGANYARPAGHVNVAPNQGDNNVPCRSDLNCGYTLDVRGGWYDAGDHGKYVVNGGIATWELLDEYERALKLGDASALGDGKLAIPERNNGVPDILDEARWEVDFLLSMQVPDGKPNAGMVHHKIHDAAWTALPTRPELDSQPRRLSPPSTAATLNMAAVAAQASRLWKTIDPAYSAKLLAAAEKAYAAAKANPNVLADPNDGTGGGTYSDNTVTDEFYWAAAELFATTGKSGYRTDVTGSSLYRGVSFNTHGFDWGSTGALGDITLALVPTDLPAADVAAIKTAITTTADSHLAQMAGMGYPAPYRTADGTYEWGSNGLVANNGVVLALAYDFTKQDKYRDGAFGAMDYLLGRNPANYSYVAGHGDQAVQNVHHRFWAHELDPSLPTAPPGALSGGPNSGLQDPTAARLLAGCAPQRCFVDDIQAYSVNEVAINWNSALAWLANWTAEKSPSAVDTTAPAAAGKPAVSGVSATGATVTWTASSDAESGIKNYDVVSVTGAARKVLATVTGTSATLTGLSPSTAYTLVVVARNGAGLSGPDSASTQFTTLPDTPAGGCAVTYSATSWSGGFTAYVTLTNTGTTAWPDWTLKFAFPAGQKVTQGWSATWSQSGAEVTAKAMSWNASVAPGKSVSIGFNGSHTGSNPAPSSFAVNGKTCS

SEQ ID NO: 84 - protein, 882 aa, Streptomyces fradiae
MRHLPHTPPPARRRSRLVGTAVMGALIAGLVAAPSGAAEPPDARAAEGPEQIVNGDFSAGTAPWWWTANSPAAVTDGELCADIPAGTSDPWDAIIGQDGVDLLAGEGYELSFTAHASVPVAIRTSVQMAVEPYTSEHLSTDRITTEPQTFTHTFTAKADNDAAQLGFQAGGHDRAYTLCLDDVSLTGGTEAPPHQPDTGSPVRVNQVGYLTEGPKGGTFVTEATQPLPWTLNAPDGSPAATGTTKVFGQDPTSRQHVHTFDFSRVTRTGEGYTVTIAGKTSEPFAIGDDLYARLRTDALAYFYHNRSGIEIDAGLVGEKYARPAGHLGKAPNQGDGDVPCQPGVCDYRLDVRGGWYDAGDHGKYVVNGGISVAQVMATYERTLTAGNADGAPLGDGRLRVPERGNGTPDILDEAAWQLDFLMKMQVPAGKTLAGMVHHKIHDAEWTGLPLLPHQDPMKRELHPPSTAATLNLAATGAQCARLFRPYDAAFADRCLTAATTAWRAALAHPAVYARADDNVGGGAYEDADVTDEFYWAAAELFVTTGQDDYRRHLLDSPLHGDAGKVFPRGGVWWGGTAGLGALSLATVDNGLTDAQLAQVRTMVTTGADRYAEDARTAAYGVPYAPEDNHYVWGSNSGVLNNMVVLGVAHDLTGNTKYRDALLGGLDYLLGRNPLNQSYVTGYGERDSRNQHHRHWAHQLDPSLPNPAPGSVAGGPNADIQDPVAQDKLKGCAPAMCYIDDIGSWATNEITINWNAPLAWVASFVDDLGGSGTTDPDPAACRVGYASDRWNGGFTSRVTVTNTGTEAISPWKLTWAFADDQRVTDHWGSRITQTGREVTAHPASWNGTIPPGGSVAFGFNGTSGGGAADPTAFALNGAPCTGG

SEQ ID NO: 85 - protein, 803 aa, Flavimobilis rhizosphaerae
MEDTTVDTVVNAPPTAVRRPLPALLALALAVSLTACDHSSPAPPPTVPATAQTSAPANTGTELLPATSFADGLGPWALYGTDAAPRTADGATCVDLPGGTTNPWDVGLTYNGLGIDASAAYHLALTASAAPATTVRVVVGEATGAYRTATELFAPLTPTAQPTEHDFTAAIALSATDGDVGQVAIQLGSATPFTFCISEASVTTRDSMPTAASDAPAVRVNQVGYLTHAAKAATVVTDAADPLPWTLDLGGMTVARGTTEPRGTDPSAGLAVHTVDLSAVTTPGTGYTLTVDGASSDTFAIGDDLYRAMRVDALSFFYPQRSGVAIDGAVAGDAYARPAGHADVGPDQGDGAVACLPAGALTVDGKDLYDGWTCEGTIDVTGGWYDAGDHGKYVVNGGIAVGQLMSTYERALHAPTGDAGALGDGTLRVPEHGDGVPDVLSEARWELEWMLTMQVPAGKDLAGMVHHKVHDDSWTGIPLLPSDDDRTRYVHRPSTAATLNLAAVAAQGARLFEPFDAAFAARLLKAARTAWAAAAAHPDLYAPNTNLHPSPGGGPYDDTELDDERYWAAVELFLTTGEDAFADALTTNPYHVGGTSEAFGPAAFDWGHVAAYARTQLATVPNALPVLAEVRRSVVDAAEATLARQATQPFGTAYVPDGGRYDWGSNGLLLNNLAVLVAAYDLGGEDRFRQGVLTGLDHLLGRNAMGISYVTGHGTRFAQNQHSRWYAAQADPSLPHPPAGTVSGGPNSDVPDPVSSSLAGCAPQRCYVDDIGAWGVNELTINWNSALAYVVSFAADQGAAGPA

SEQ ID NO: 86 - protein, 915 aa, Lachnospiraceae bacterium
MKTRSTIRRAVALLFLALFCLSRPWPALTVLAGEQLGETDFDDGKGLPWHIVESEPAKMDFELSDGQYIITIINPGGASQGGEDRWDCQFRHRGLTIVSGHQYRVRYELTASNSGKYYTKIGNLAGDTEIWHNMSNGYDLDATWDPIPVNAGETKRVDLTFTAAQGMEVAEWAFHLGGDGQYTPGGCFPAGTVIRFDNMSLEDLSSDENDYVEEEPWARALILTNQLGYIEDAAKKATLLNDSDKPVSFSLIDENGRVAYEGKSIPYGYDADSGDMVHVLDFSEADAPGEYCIEAEDGARSRSFSIGVSSVYSSLLYDSLNYFYQNRSGIEIESSLICSGDGSELSRAAGHTPDLASVDQCWGYSASSGTQDVSGGWYDAGDHGKYVVNGGISLWLLQNMYEAALYCGTEAAYADGTMSIPENGNSFPDLLDEARWEMEWMFTMLVPDGSCRDMVYHKVHDIKWTGLGVAPADDPQERILKPPTTAATLNVAACAAQASRLWAGIDKAFAEECLDVAVRCYDAACDHPDMYAPLDESVGGGAYGDDDVSDEFYWAATELWLTTGEENYHKDMKSSPWYLAVPETMDGGESVGACGSFDWGHTAALASMSICLNEDACDSSEYKKTKDNLLETAEHYLSLCDEQGYGQPYAASSIAYNDPDNGYIWGSNSVVADNAVILAYAYLISGDSAYFDGAVSGMDYLLGRNPMDYSYVTGYGSHAVTYPHHRFWAKQIDDSFPMAPAGVLSGGPNSGMQDPWVRGMGWKKGTIPPAKCYLDHIEAWSVNECTINWNAPLAWLSGFLTEYSPGGITPGAGSFGSGVSLQTSSEERESYTAEKTDRGSVTASGKKSSRDRENDRDSEREEGSESNVLLIVIVIAASVLLLAVAVMIFVYKMAKLKALSGADPKNKGPDQPLGA

SEQ ID NO: 87 - protein, 700 aa, Streptomyces sp.
MKRRRATLLAATALLAAALTALPASPAGADEVEQVRNGTFDTTTTPWWTTSNVTAGLSDGQLCADVPGGTTNRWDAAVGQNDITLVKGESYKFAFTASGSPAGHVARAIVGLSVSPYDTYYEISPELSETGNSLAYTFTSPVDTTQGQVAFQLGGSADGWHFCMDDVSLLGGVPPEVYEPDTGPRVRVNQVAYLPGGPKNATLVTASTAVLPWELKSAAGAVVAHGRTVPRGTDVASGQNVQSIDFGAYRGRGEGYTLVADGETSRPFDIGTAAYERLRLDSLKYYYTQRSGIAIRDDLRPGYARPAGHLDVAPNQGDANVPCQPGLCDYTLDVTGGWYDAGDHGKYVINGGISTWELLSTYERSLLARTGQPGKLRDGTLAIPESGNKVPDVLDEARWELEFLLKMQVPDGQPLAGMAHDKVHDEQWTGLPLLPSQDPQKRELHAPTTQATLNLAATAAQAARLYRPYDREFAAKALKVARKAWAAALAHPAVYPDPNDGTGGGAYPDDNATDEFYWAAAELYLTTGERQFADHVVNSPLNTADIFGSLGFDWARTAALGRLDLATVPSRLPGRDKLRRSVIEGADTYLATLRSEPYGMPYAPPGNLYDWGSNSQILNNAHVIATAYDLTGAAKYRDGALQSMDYILGRNALNMSYITGYGEANSHNQHSRWYAHELDPTLPNPPDGTLAGGPNSSIKD

SEQ ID NO: 88 - protein, 795 aa, Kineococcus sp.
MHPARKRRARALTAATALLAPVSLLLTPTAAHAADQIVNGAFDQGTTGWTIYPNPSVQDGAGCATVPASTAAYGAAIQQEIPLVAGESYKLTFRARTTPATEGPVRVVVQGGADVNHQQFLPADKPAFASAFGTHEYYFTAERDYPNAQLAFQQDVVNPVGYTLCVDDVVLEGGAERPPYVPETGPRVRVNQETYLPDGPKGATLVTDATTSLPWELKDAAGTTVAKGTTTPRGVDPTAGLNVHTIDFSRYAEGGTGYALVADGETSHPFDIGEAAWERLRDDAMTFFYTNRSGTPISDELTPGYGREAGHVGVAPNQGDTAVPCQSLDDDSQEILVAQGDEPWTCDYTTDVTGGWYDAGDHGKYVVNGGIAVAQLLQQYERSLHAPTADRGALADGTLRIPEAGNGVPDVLDEARWELEWFLKMQVQPGRPLEGMVFHKVADVDWTGLPLDPAADPQPRVLYRPSTAATLNVAATAAQGARLFAEFDPAFSERLLAAARTAYAAARANPELYAPAPDATLDPNPGSGPYDDDDVSDEFYWAAAELYLTTGEPQFRKDVLRSPVHTADVFPAGGFDWGHVGALGRLDLATVPSRLGKHELRQVRRSVTEAADRLLAAQATQPFGQAYAPEDGQYAWGSNAQILSNLQVLGTAFDLTGKDAYRDGVVRSTDYLLGRNALNNSYITGYGDVFSRNQHSRMYANQLDPASPNPPAGTVAGGPYSPTATSGDPITAALFPDGCVAQFCYVDDIGSWSTNEITINWNAPMAWVASFVADQDSGCDAGRGHGRGHGGGHRG

SEQ ID NO: 89 - protein, 1117 aa, Cellulomonas persica
MVSWFPRRARTAALSAGLTATALVATAAAAPLAFADPVESPHDFTDGTQGWYSYGGDSSSSIVDGEFCVVVPAGTTNPWDAAAQHDGVTFAAGDTYTVSFSAHATVPVSVNLRAGIGWPDDVSSTVALTEDEQDYAFTWTPGFSGDGNTSFQLGAQAQDYTFCLDDFQISGEVELIPDTSFDGVLPEGWSANGWNVTSEPGEGEPLCFEVLDGSGQYAGLVLNGLPIEADANYRLTYTASSSNAATIRTVIGENAEPWRTAFVDNTQLTTDLQEHVLAFTSPVTFPAESSDPAVGVGQVALQMGARGDFTFCITSLSLQKVATPPPPYEPDVRGPIRVNQLGYLPGGPKNATVVSDASEPLDWQLQDAAQQVVASGQATPAGLDPSSQLDVQTIDFSAFQTPGTGYTLVVGEDRSDPFAISATLYDQLRYDALNYFYPVRSGIAVDVPDDRYDRPAGHVSGPDGAVNKGDLDVACLTAAEDGASWSYGTWTCPQGYSLDVVGGWYDAGDHGKYVVNGGISVAQLLGTYERTLHRKNVVAGALGDGTLDVPADESGNKVPDVLDEARWQLEFLLAMQVPAGSGMTVSATDTRSLDGLVHHKIHDVGWTGLGLLPSDDPQQRRLHRPSTAATLNLAAAAAQGSRLFEKYDKAFAKELLKAAKTAYAAAERVPDLYAPVTAGQNGGGPYDDTDVSDEFYWAAAELFLTTGDPAFRKDVLASPDHTGDIWSRTGFSWGSVAPLARMDLATVPSKLPKKDVDAVRASVVAGADTYLAWQEAEAFGHAYPGQADGSYEWGSNSAVLNNQVVLGTAYDLTGDPRYARAVLESMDYLLGRNALNNSYITGYGTQYSSHQHSRWLVPPLPGTISGGPNSQRGTWDPVMNRLYPAGHECAPQMCYVDDIEAWSVNELTINWNAPMSWVASFVADVAAKGLPAAPVVTTQPQDATVAPGATASFVVAATGSPTPTVRWEQRDAKGRWKPVHGATSTTLTVKASPATDGAQFRAVFTNPSGSATTDAATLRITRQAPTITVHPASASGKQGTKVTLRAAATGYPTPRIQWQVLEKGSTSWVDVRRACGTSLQFVIGPPTDGDRYRAVFTNTAGSATTREATITLARGPR

SEQ ID NO: 90 - protein, 1176 aa, Cellulomonas fulva
MAAPLASAAPIDQVHDFSDGPQGWYSYPDTAPSSVVDGEYCVVVPGGSANPWDIAIQHDGLTFEAGTDLTASFRAHATADVSVNLRAGTGWPDDVASTVAVTTVAQDFAFSWEPGFSGDGNVSFQLGGKSADYTLCLDDFSISGAQELVPDTSFDGVLPEGWTNEGWTVTSEPGDGEPLCFEVPGTSGTYSGLVLNGLPIEEGGNYELRYTASASNGATIRTVVGENAAPWRTAFVDSTELSTELTEHVLPFTSGVTFPAQSSDPAVGVGQVALQMGDRGDFTFCITSLSLRKVATPPPAYEPDTRGPVRVNQVGYLTNGPKSATVVSDATDPLAWQLQDADEHVVAEGTATPAGTDPTSQLAVQTIDFREFTTPGEGYTLVVGDDRSDPFAIGADLYDQLRYDALNYYYPVRSGIAVDVPDDTYDRPAGHVDGPDGAVNKGDVDVACLTAADDGASWSYGSWTCPEGYARDVVGGWYDAGDHGKYVVNGGISVAQLMSLYERSLHTDHATDGALADGTLDVPADESGNGVPDVLDESRWELEFFLAMQVPQGSGMTVSADDDRSLDGLVHHKIHDVGWTGLGLLPSADPQQRRLHRPSTAATLNVAATAAQGARLFREYDADFADRLLEAAQTAYAAAQRVPDLYAPASAGSNGGGPYDDTDVSDEMYWAAAELYLTTGDDEYEADVLASEHSDDDIWSRSGFSWGSVAALGRLDLATVPSDLPTRDEVRASVVEGAEKYLGWQEAEAFGTAYPGSDDGQYEWGSSSSVLNNQVVLATAYDLTDDPRFSDAVAESMDYLLGRNALNNSYVTGYGTQYSTHQHSRWLVPPMPGTVSGGPNSRRGTWDPVMNTLYPAGHECAPQACYVDDIEAWSVNELTINWNAPLSWVASFLADLGEGVAPQAPAFTQQPHGVTVALGATATFTVQVTGTPTPTVAWQWRTGAGTWQTIPRATGTTLSVVASAATDGRQYRAVLTSTAGTATSSVATLVVKAQRPVVTKHPTSASAAVGRSVTLRASASGYPTPTVRWQLRKPGSSSWKNVAGGTRTWLTVRLSTANTGTQYRAVFTNRGGSATSRAATVRLAQAAPRFTSHPASTTVRAGQKATFTVTVAAYPKATLTWYVRTKGSASWKAVTGARWSTLSVTATRALDGAQYRAVARSSAGSAWSKVATLTVR

SEQ ID NO: 91 - protein, 1103 aa, Cellulomonas sp.
MVSFLPGRRRATWAGAATAGLATLALVVAPMAAANPIGEVHDFADGPQGWFSYGGTSSSSVDDGELCVVVPAGTTNPWDVAAQHDDVTLAADSPVTVSFRAHATADVSVNLRAGIGYPDDVSSSVALTSAPTDYSFTWTPGFSGTGNTSFQLGGQASDYTFCLDDFSISGDDELLGTTTFAGDTLPAGWSLPADWAVTSAAGDPAFCVAVPASAGQYDGLVYNGVPVEDGGNYALSFTASASNGATVRALVGENGGAYRTAYVANPALTAELASYDYPFTSSLAFPADEGAVGQVALQLGARGDYTFCVSEVSLRKTATPPPAYEPDTRSRVRVNQVGYLPQGPKRATVVTDAAAPVAWELHAAGGALVASGKAALAGTDPTSGLKVQTVDFSTVTAKGAGFTLVADGETSDPFAIKTDIYAQLRYDALNYFYPARSGIAIDAPGTVYDRPAGHVSGPDGAVNKGDLDVACLTAADDGASWSYGTWTCPAGYARDVVGGWYDAGDHGKYVVNGGIAVQQLLSTYERTLYATDAAKGALKDGTLEGPADESGNGVPDVLDEARWELEFFLAMQVPAGSGMTVSSTDSRSLDGMVHHKIHDVGWTGLGLLPSDDPQQRRLHRPSTAATLNVAATAAQGARLFEKYDKAFAAKLLAASRTAYTAALRVPDLYAPASAGANGGGPYDDTEVSDEFYWAAAELYLTTKSSTYKKDVEKSAHRTDDIWTEGGFNWGSTAALGRLDLATVPSDLRTQARTSVVAGAKKYLTWQKAQAFGTAYPGSNGTYDWGSNSMVVNNQVVLATAFDITRDKKFSRAVLESMDYLLGRNALNNSYVTGYGTQFSSHQHSRWMVPPPNGSLAGGPNSLRSTWDPVMNTLFPESNPCTPQRCYVDDIEAWSVNEITVNWNSALSWIASFVADQGAAPQPVAPKITKQPTSVSGALKAKVTLSAAASGVPAPTVRWQRRAPGATAWKDVRGATSTTLKVVMTRAEHRAGYRAVFTSSAGSATTATATVTLRKARPVITSQPKDVTVRSGARATFRVAYDAYPAAHVQWWAKKGKTWVKVPGARAQTLTVKVTGTSSKHYRVVVKSSQGQVTSRTAHVTIRR

SEQ ID NO: 92 - protein, 927 aa, Herpetosiphon giganteus
MSADHSRTVIGIRSTRSFWARGLFVLWLIIALGCQQAIVPATSQIHAQPPQTVGNLLQNGDFSAGLAPWWATNTVTADAASGALVATITNAGSNPWDAIVGQNGLNLQAGQTYTVTFRIRAASTGTAVLKLQKEASPYTNYFSQDVALTTSDQTHQFVFTSGFDDAGAAFQFQMGGQGNNVLTIDDVQVLGETGPAEPSSNLVQNGNFVGGIEPWWTSGDLSIDTADGACLTINTAGSNPWDVQLGQHTIALQAGASYQLKFAAKSTTPVTLPVRLQQNGEPYTGYFAADPGLNAAWTEFVFTFTSAYSDAASLLFQIGGLGTPTICFNHVSLEMLETGIRVNQASYLPTMSKVASLAHPATEPLSWELHNAADTVVLSGQTTVYGEEAASAEHVHLIDFSSFQIVGEGYYLQVGSEQSYPFAIEDGLYLRLKYDALAYFYHNRSGIAITMPYAGDEQWTRPAGHIGVAPNQGDLAVTCFAGTDNQGQTWPGCDYELDVSKGWYDAGDHGKYVVNSGISVWTLLNQYERAQQRGAESLAQFADGSMNIPENNNGVPDLLDEVRWNMEFMLGMQIPTTAPVSKTGMVHHKVHDAQWTGLPMAPHDDPQMRYLYPPSTAATLNLAATAAQCSRIWRGIDEAFADRCLVAAERAWQAAVAYPNEFASDHFAGGGGYGDRTLNDEWYWAAAELYVTTGSATYREAIEESPYYLKLDIGGGSAMNWGSVASLGTLSLALVPNHLSSADRQTARAAVIAAADQFVAAQQASGYGIPYNPGSQYPWGSNSSILNNMIVIGLAGDFTGETSYADAISQGMDYLLGRNPLNRSYISGYGSVSLTNPHHRFWAKQINPEYPGTPPGVVAGGPNSGIEDPYAQAELTGCAALKCYVDHIDSWSTNEVTINWNSPLAWVAAYLDDYATGSVQYLPLITK

SEQ ID NO: 93 - protein, 991 aa, Ruminococcus sp.
MSKSFKRVTSGLLALSVVASMGSMNAFAGQQLGQNDFDSGIGLPWHTCETNPAKQEFDISGGTYNVKIVNNDGPEDRWDLQLRHRGLKIVSGHKYKLHWEVNSTSAGEIYTKIGDYDSVEAWHNNMDDSKGFDQTWDCVPIKAGDNEFEVEFTAQKSIDVAEWAFHYGGKGQYQKVDCFPNGTELKFDNLSLIDLTSDENDYDTTNEMGVVRPESNVRLNQVGYYTNLNKKASYVTDASSSLTFEVRDAKSGAVKYTGKTTVKGEDPDSGTGTTSGSKLKDSGKYVHILDFSSVKDAGTYKIFVKDTTGVSGTQVLLAKGANDTKLSGDKLMWTNPQTKKTYVMNESHDFVISDDVYGTQLLKDSINYYYQNRSGVPIESQYITSGDKSELSHTQYGHNPDTAYVQSEWKKAYASEFDGDKGKTIDGTGGWYDAGDHGKYVVNGGISVWTLQNAFEFTKKNGNTDKWTNKTIAIPENGDKNPDILDEARVELEWMFKMIDEDGFVYHKLHDHKWTGLATHCWDYEKKWKTTRIVKPATYAATFNMIACAAQAARLWEPYDKSFAQECLQHAEASWKAIYAKKDNWNVKEGDSSKDSYFAPLDQAIGGGAYGDTYVVDDAYWAACELYSTTGKSDYLSFLKSYKNPNDKTENDKAYSLTYNLGGGENNGSFSSFNWGCTSGLGTLSLYLSDKVSSSEKATITKSITAVADEYLKEMAKQGMGIPYRGATFTDAINIGEGVSVTGYEWGSNSFVINNAMVLAYAYDATKATASDKGGKYINGASEALDYIFGRNGLGFSYVSGYGDKTLEWPHHRFWSKGVDPAFPKAPNGVLSGGPGAGMQDPYIGGLGYKRGTLANQKCYVDSAEAWSVNEVTINWNAPLVWMTTFMLDEAPKFNGGTPDPVVVQCPTVKAEVQGKQFRLKWEAVKGATAYAVALYQNGAWRCKAQVSGSTLTYTSPKGVKSGKYQVAICPKVNGQWVNKSIKSKSITVTI

SEQ ID NO: 94 - DNA, 2580 bp, Aspergillus welwitschiae
  source 1..2580: mol_type = genomic DNA
atgaggttcactttgatcgaggcggtggctctgactgccgtctcgctggccagcgctgatgaattggcctactcccctccgtattacccctccccttgggccaatggccagggtgactgggcggaagcataccagcgcgctgttgatatcgtctcgcagatgacattggctgagaaggtcaatttgactacgggaactggatgggaattggaattatgtgttggtcagactggaggtgttccccgattgggaattccgggaatgtgtgcacaggatagccctctgggtgttcgtgactccgactacaactctgcgttccccgccggtgtcaacgtggccgcaacctgggacaagaatctggcttaccttcgtggccaggctatgggtcaggagtttagtgacaagggtgctgatatccaattgggtccagctgccggccctctcggtagaagtcccgacggcggtcgtaactgggagggcttctcccccgacccggccctcagtggtgtgctctttgcagagacaatcaagggtattcaggatgctggtgtggttgcaacggctaagcactacatcgcctacgagcaggagcatttccgtcaggcgcctgaagctcaaggctacggattcaatattaccgagagtggaagcgcgaacctcgacgataagactatgcatgagctgtacctctggcccttcgcggatgccatccgtgcaggtgccggtgctgtgatgtgctcgtacaaccagatcaacaacagctatggctgccagaacagctacactctgaacaagctgctcaaggctgagctgggtttccagggctttgtcatgagtgattgggcggctcaccatgccggtgtgagtggtgctttggcgggattggacatgtctatgccgggagacgtcgattacgacagtggcacgtcttactggggtaccaacttgaccatcagtgtgctcaacgggacggtgccccaatggcgtgttgatgacatggctgtccgcatcatggccgcctactacaaggtcggccgtgaccgtctgtggactcctcccaacttcagctcatggaccagagatgaatacggcttcaagtactactatgtctcggagggaccgtatgagaaggtcaaccagttcgtgaatgtgcaacgcaaccatagcgagttgatccgccgtattggagcagacagcacggtgctcctcaagaacgatggcgctcttcccttgactggaaaggagcgcttggtcgcccttatcggagaagatgcgggttccaatccttatggtgccaacggctgcagtgaccgtgggtgcgacaatggaacattggcgatgggctggggaagtggcactgccaactttccctacttggtgacccccgagcaggccatctcgaacgaggtgctcaagaacaagaatggcgtattcactgcgaccgataactgggctattgatcagattgaggcgcttgctaagaccgccagtgtctctcttgtctttgtcaacgccgactctggtgagggttatatcaatgtcgacggaaacctgggtgaccgcaggaacctgaccctgtggaggaacggcgacaatgtgatcaaggctgctgctagcaactgcaacaacacgatcgttattattcactctgtcggcccagtcttggttaacgagtggtacgacaaccccaatgttaccgctattctctggggtggtcttcccggtcaggagtctggcaactccctcgccgacgtgctctacggccgtgtcaaccccggtgccaagtcgcccttcacctggggcaagactcgtgaggcctaccaagattacttgtacaccgagcccaacaacggcaacggagcgccccaggaagacttcgtcgagggcgtcttcattgactaccgcggatttgacaagcgcaacgagactcctatctatgagttcggctatggtctgagctacaccaccttcaactactcgaaccttcaggtggaggttctgagcgcccctgcgtacgagcctgcttcgggcgagactgaggcagcgccgactttcggagaggtcggaaatgcgtcggattacctctaccccgatggactgcagagaatcaccaagttcatctacccctggctcaacagtaccgatcttgaggcgtcttctggggatgctagctatgggcaggatgcctcagactatcttcccgagggagccaccgatggctctgcgcaaccgatcctgcctgccggtggtggtgctggcggcaaccctcgcctgtacgacgagctcatccgcgtgtcggtgactatcaagaacaccggcaaggttgcgggtgatgaagttcctcaactgtatgtttctcttggcggccctaacgaacccaagatcgtgctgcgtcaattcgagcgtatcacgctgcagccgtcggaagagacgcagtggagcacgactctgacgcgccgtgaccttgcgaactggaatgttgagacgcaggactgggagattacgtcttatcccaagatggtgtttgtcggaagctcctcgcggaagctgccgctccgggcgtctctgcctactgttcac

SEQ ID NO: 95 - DNA, 2583 bp, Aspergillus luchuensis
  source 1..2583: mol_type = genomic DNA
atgaggttcactttgattgaggcggtggctctcactgctgtctcgctggccagcgctgatgaattggcttactccccaccgtattacccatccccttgggccaatggccagggcgactgggcgcaggcataccagcgcgctgttgatattgtctcgcagatgacattggctgagaaggtcaatctgaccacaggaactggatgggaattggagctatgtgttggtcagactggcggggttccccgattgggagttccgggaatgtgtttacaggatagccctctgggcgttcgcgactccgactacaactctgctttcccttccggtatgaacgtggctgcaacctgggacaagaatctggcatacctccgcggcaaggctatgggtcaggaatttagtgacaagggtgccgatatccaattgggtccagctgccggccctctcggtagaagtcccgacggtggtcgtaactgggagggcttctcccccgacccggccctaagtggtgtgctctttgcagagaccatcaagggtatccaagatgctggtgtggtcgcgacggctaagcactacattgcctacgagcaagagcatttccgtcaggcgcctgaagcccaaggttatggatttaacatttccgagagtggaagcgcgaacctcgacgataagactatgcacgagctgtacctctggcccttcgcggatgccatccgtgcgggtgctggcgctgtgatgtgctcctacaaccagatcaacaacagctatggctgccagaacagctacactctgaacaagctgctcaaggccgagctgggtttccagggctttgtcatgagtgattgggcggctcaccatgctggtgtgagtggtgctttggcaggattggatatgtctatgccaggagacgtcgactacgacagtggtacgtcttactggggtacaaacctgaccgttagcgtgctcaacggaacggtgccccaatggcgtgttgatgacatggctgtccgcatcatggccgcctactacaaggtcggccgtgaccgtctgtggactcctcccaacttcagctcatggaccagagatgaatacggctacaagtactactatgtgtcggagggaccgtacgagaaggtcaaccactacgtgaacgtgcaacgcaaccacagcgaactgatccgccgcattggagcggacagcacggtgctcctcaagaacgacggcgctctgcctttgactggtaaggagcgcctggtcgcgcttatcggagaagatgcgggctccaacccttatggtgccaacggctgcagtgaccgtggatgcgacaatggaacattggcgatgggctggggaagtggtactgccaacttcccatacctggtgacccccgagcaggccatctcaaacgaggtgctcaagaacaagaatggtgtattcaccgccaccgataactgggctatcgatcagattgaggcgcttgctaagaccgccagtgtctctcttgtctttgtcaacgccgactctggcgagggttacatcaatgtcgacggaaacctgggtgaccgcaggaacctgaccctgtggaggaacggcgataatgtgatcaaggctgctgctagcaactgcaacaacaccattgttatcattcactctgtcggcccagtcttggttaacgaatggtacgacaaccccaatgttaccgctattctctggggtggtctgcccggtcaggagtctggcaactctcttgccgacgtcctctatggccgtgtcaaccccggtgccaagtcgccctttacctggggcaagactcgtgaggcctaccaagattacttggtcaccgagcccaacaacggcaatggagccccccaggaagacttcgtcgagggcgtcttcattgactaccgcggattcgacaagcgcaacgagaccccgatctacgagttcggctatggtctgagctacaccactttcaactactcgaaccttgaggtgcaggttctgagcgcccccgcgtacgagcctgcttcgggtgagactgaggcagcgccaacttttggagaggttggaaatgcgtcgaattacctctaccccgacggactgcagaaaatcaccaagttcatctacccctggctcaacagtaccgatctcgaggcatcttctggggatgctagctacggacaggactcctcggactatcttcccgagggagccaccgatggctctgcgcaaccgatcctgcctgctggtggcggtcctggcggcaaccctcgcctgtacgacgagctcatccgcgtgtcggtgaccatcaagaacaccggcaaggttgctggtgatgaagttccccaactgtatgtttcccttggcggccccaacgagcccaagatcgtgctgcgtcaattcgagcgcatcacgctgcagccgtcagaggagacgaagtggagcacgactctgacgcgccgtgaccttgcaaactggaatgttgagaagcaggactgggagattacgtcgtatcccaagatggtgtttgtcggaagctcctcgcggaagctgccgctccgggcgtctctgcctactgttcactaa

SEQ ID NO: 96 - DNA, 2583 bp, Aspergillus sclerotioniger
  source 1..2583: mol_type = genomic DNA
atgaggttcagttggatcgaggtggcggctctgacagccgcctcggtggtcagcgctgatgaattggcgtactctccccctttctacccttctccctgggccaatggcaagggtgactggacagacgcgtacaagcgtgcggtcgacattgtttcgcagatgacactggccgaaaaggtcaatctgacaacgggaactggatgggaattggaaaagtgtgtcggtcagaccggcggtgttcctcgattgggaataccgggcatgtgtggtcaggatagtcctctgggtgttcgtgactctgactacaactcggctttccctgctggcatcaacattgccgcttcctgggacaaggacctcgcgtacctccgtggcaaggccatgggccaggagttcagtgacaagggtgctgatattcaattgggccccgttgctggtcccctcggtagaagtcccgacggcggtcgtaactgggaggggtttgctccagacccggccctgactggtgtgctctttgcagagaccatcaagggtatccaggatgctggcgtggttgcgacggctaagcacttcatcggctatgagcaagagcatttccgccaggcacctgaggctcaaggatatgggtacaacatcaccgagagtggcagcgcgaacatcgacgataagaccatgcacgagctgtacctctggccctttgcggatgctatccgcgcaggtgttggcgcggtcatgtgctcctacaaccagatcaacaacagctacggttgccagaacagctatgctctgaacaagctccttaagtctgaactggaattccagggcttcgtgatgagcgactgggaggcccaccacagtggagtcggtgctgctttggccggtctggacatgtctatgcccggagatgtcacctacgacagtggcacgtcttactggggtgccaacctgaccatcagtgtcctgaacggcacggttccccagtggcgtgttgatgatatggctgtccgtatcatggccgcctactacaaggtcggtcgggatcgtctgtggacgcctcccaacttcagttcatggacccgcgatgaatacggctacaagtactactattcctccgagggaccgtatgagaaggtcaaccagtttgtcaacgtgcagcgcaaccatagcgggttgatccgccgtattggagctgacagcacggttcttttgaagaacgagaacgttctgcccctgactggcaaggagcgcctggtcggtcttatcggagaagacgcgggctccaacgcttacggcgccaacggctgcagtgaccgtggatgcgacaacggtactctggctatgggctggggcagtggtacctcgaactacccttacttgatcactcccgagcaggcgatccaggctgaggtgatcaagaacaagggcaatgtatttgccgtgaccgacaactgggccattgaccagattgaggcactcgccaagcaatccagtgtctctcttgtctttgtcaacgccgactctggtgaaggttacatcgatgtcgatggcaacatgggtgaccgcaacaacattacgctctggaggaacggtgacaacgtggtcaaggccgctgccaacaactgcaacaacaccattgtcatcatccactccgtcggcccggtcctcgtcaccgagtggtacgaccaccccaatgtcaccggcatactgtgggctggcttgcccggccaggagtctggcaactcgctcgccgatgtgctctacggccgtgtcaacccgggtgccaagtcgcccttcacctggggtaagaccagggagtcctaccgcgactacctgatcaccgagcccaacaatggcgatggagccccacaggaagacttcaccgagggcatcttcatcgactaccgcgggttcgacaagcgcaatgagaccccaatctacgagttcggctatggcctgagctacaccaccttcaactactcgaatttgaacgtgcaggtcctgaatgcctcgtcgtacactccttcttctggcgagactgaggctgccccgacttttggagagatcggcaacgcgtcggactacctctaccctagcggactgaagaagattaccgacttcatctacccctggcttaacagcacggacctcctggaagcttccggcgacgccagctacggtctgaacgcgtcggagtatatccccgagggaggcaccgatggctctgcccagccgatcctggctgctggtggtggccctggcggtaaccccggtctgtatgacgagctgattcgcgtttcggcgaccatcaagaacaccggcaagcttgcgggtgacgaggttcctcaactgtacgtttcgcttggaggtcccgatgacgccaaaattgtgttgcgcaagttcgaccgcatctctctcaagccatcgcaggaggttgagtggagcacgactctgacacgacgcgacctggcaaactgggatgttgcggcgcaggactggaccatcacgtcttatcccaagacggtgtatgtcggtagctcttcgcggaagctgccgctccgcgcgtcactgcctactgtccaatag

SEQ ID NO: 97 - DNA, 2586 bp, Aspergillus tanneri
  source 1..2586: mol_type = genomic DNA
atgaggtttggttggtttgaggcggcggtcgtgaccgctgcctcagtggtcagtgcccaggatgatcttgctttctctcctccgtactatccttccccgtgggccaatggccagggagaatgggccgacgcgtatgaacgtgccgtcgacatcgtctcccagatgacgttggcggagaaggttaacctcactactggaacagggtggatgttggataaatgtgtgggtcagacaggaagtgttcccagactcggactattaagtctctgcttgcaagacagtcccttgggtattcggttttcggattacaattcggcattccccgcaggtgttaatgtcgccgcaacatgggacaagcaactggcgtacctccgcggtagggcaatgggtgaggaattcagcgacaagggaatcgacgttcaattaggccctgctgctgggcctctcggcagacaccccgatggtggtcggaactgggaaggtttctcccctgaccctgcccttaccggtgtacttttcgcagaaaccataaaaggcatccaggatgctggtgtgatcgcgacggccaagcattacattttgaatgaacaggagcatttccgtcaagttcccgaagcccttgcggctggattcaatatttcggactccctgagctccaatctagacgacaaaaccatgcatgagctgtacctctggcccttcgcagatgcagtgcgcgcgggtgtgggtgctgtgatgtgctcctacactcagatcaataacagttacggctgccagaatagcgaaactctgaataaacttctcaaggcggagcttggcttccagggttttgtcatgtcggactggagtgcgcaccacagcggtgtcggctctgccttggcggggctggatatgtcaatgcccggggatatcgccttcaatgacggcaattcgtacttcggggcaaacttgacgattggcgtcctcaacggaaccatccctcagtggagagtggacgacatggctgtccgcatcatggctgcttactataaggttggccgggatcgccaccacgcgcctcccaacttcagctcatggaccagggacgaatacggctacgagcacgctgcagtttcggagggcgcgtatgaaagggtgaaccaatttgtcgacgtgcagcgggaccatgcggaaatcatccgtcgtgtgggtgccgagagcattgttctcttgaagaacgagaacgctctgccgttgagtgggaaggagaagaaggtcgttatcctcggagaagatgcgggatccaacccttggggtgccaatggctgcgaaaaccgtggatgtgacaatgggacccttgccatggcttggggcagtggcactacagagttcccctacctcgtcactcccgaacaggcgatccagaacgaggtcctccgaggtcgtggtaatgtatttagtgtcaccgacaacggggctctgagtcagatggcggcgcttgcctctcaatctagtgtcgctcttgtctttgtcaatgccgactcgggtgagggattcatcaccgtggatggaaacgagggagatcgcaagaacctcactctctggaagaacggagagaacgtgatcaaaaccgctgcccaaaactgcaacaacaccgtcgtgatcatccactccgtcggagccgttctggttgacgaatggtatgaccaccccaatattaccggcattctttgggcgggtctgcctggtcaggagtccgggaactccctcgccgacgtgctgtacggccgtgtcaaccctggcggtaagacccccttcacctggggtcgaacccgcgaatcgtatggggcacccctggtcaccgatgcgaacaacggccacggtgcgccccagtcggacttcgaagaaggggtgtttattgactaccgtcatttcgacaagttcaacgagactcccatctacgagtttggctacggccttagttacaccactttcggctactcgggactgcaaattgagcccctgaacgcgcccaagtacgtccccaactccgggaagaccgaagcggcgccaactttcggcgatgttgcagaggtctcagactatgtgtatccaaagggactgcggagaatccgtgagttcatctacccctggctgaattccaccaatctgaagaagtcctccggcgatgccacttatggaggggaggactcgacgtacatccccgatgacgccaccgatggttctcctcaggaccttctgcctgccagcggtggtcccggaggcaatccaggtctctaccaggatctggtgcgggtgtcggttacgatcacgaacacgggcaacgtggccggctacgacgtgcctcagctttatgtttcccttggtggccccaacgagccgaaggtggtactccgcaaattcgatcgatttaagctggatccgtcgcagcaggtggtgtggtcgaccacgctgaatcgtcgcgacctgtccaactgggacgtgacggggcaggactgggtcatcaccgagtatcccaagaaggtcttcgttggtagctcgtcccggaaattgccgctacatgcttccttgccggagatggagtaa

SEQ ID NO: 98 - DNA, 2586 bp, Aspergillus glaucus
  source 1..2586: mol_type = genomic DNA
atgaagctcggctggttggagtttgttgccgtcacggcctcggtggcccaggctaaggacctcgcgtactctcccccctactatccctcgccatgggccgatggtcaacccgccgaatggtccaacgcgtacaaacgcgccgtcgacatcgtctccaacatgactttggcggaaaaggtcaacttgaccactggtactggctggcaattggaagaatgtgttggtcagactggtagtgttccacgacttggtatctggggtatctgtttgcaggactcgcctcttggtatccgttatggcgatcacagctctggcttccccgctggtctcaacgtcgccgcgacctgggaccgcaagctcgcctacctccgtggtgaggctatgggtcaggaattcagcgacaaaggaattgacgtccaattgggtcccgttgctggccctatcggcagatctcccgacggtggtcgcaactgggagggtttcgcccccgacccggttctgaccggtgtgctcatggccgagactatcaagggtattcaggatgccggtgtcatcgctactgctaagcactacatcggcaacgagcaggagcatttccgtcaggtttccgaggctctcgactatggttacaacatcactgagactgccagctccaacatcgacgacaagaccatgcacgagctctacttgtggcctttcgccgatgctgttcgtgctggtgtcggttccgtcatgtgctcgtacaaccagatcaacaacagctatggttgccagaacagtcatttgctgaacaagctgctcaagcacgagctgggcttccagggcttcgttatgaccgactggggtgctcaccatagcggtgtcgcctccacccttgctggtactgacatgtcgatgcccggtgatatctcgtttgacgatggtatgtcgtacttcggtcccaacctgactgttgctgttctcaacggtactgttcccgagtggcgtgtcgacgacatggccgttcgtatcatgtctgctttctacaaggttggccgtgaccgccagcgcacgcctcccaacttcagctcctggaccaacgacgagtacagctacgcccactccgccgtccaggaaggctggaagcaggtcaaccagcacatcaatgtccagcgcaaccactccgagatcatccgtgaggttggcgaagccagcactgttctcttgaagaacaagggtgctcttcctctgactggcgacgagggctccgttggtattctaggcgaagatgccgggtccaacgcgtatggtgccaacggctgcgaggaccgtggatgcgacaatggtacccttgcgatggcctggggtagcggttccgccgaattcccttacctggttactcccgagcaggctatccagaacgagattctcaaccgggacgtcaagcaccccgtcttcgccgtcactgacaactgggctttggatcagatggcctccattgcctctcaatctgatgtttctcttgtcttcgtcaacgccgatgctggtgagggtttcctcgtcgtggatggtaatgagggtgaccgcaataacatcaccctctggaagaacggtgagaatgtcatcaagactgtcagcgagaactgcaacaacaccattgtgatcatgcacaccgtcggacccgtcctcatcgaccagtggtacgacaaccccaacatcaccgccattgtctgggctggtctgcctggccaggaatccggaaacgccattgccaacgttctctacggccgtgtcaaccctggtggcaagagccccttcacctggggtaagagccgcgaggcatacggtgccccgctcctcaccgagacaaacaacggcatcggcgctcctcaggtcgactttactgagggtcagttcatcgactaccggcgcttcgacaagtacaacgagacccctatctacgagtttggctacggtctgagttataccaccttcaagtactccaacctccacgtccaggccctgaacgcctccaagtacgttcccaccactggcaagaccagtgccgcgcccaagcttggtgaggttggcaaggcttcgaactacgtcttccccaatggattcgagcgcaccaccaagttcatctacccctggttgaactcgaccgatctgaagaagtccgccaacgaccccgagtacggcctcgagatctcaaagtacatccccgagaacgctcaggacgaatcctcccaggcccgtttgccagccagcggcggccaaggaggcaacaccggtctctacgacgaactcttccgcgtctccgccacgatcaagaacaccggcaaggtcgcaggtgacgaagttccccagctgtacgtctctctcggcggccccaacgagcccaaggttgtcctgcgcaactttgaccgcatcgccctccagcctggtcaggaggtcgtgtggtcgaagactcttacccgtcgtgacctttccaactgggacgttgccgctcaggactgggctatcacgcagcataccaagaaggtgtttgttgggagctcgtcgcgcaagctgcctttgcgggcttcgttacctcgcgtgcagtag

SEQ ID NO: 99 - DNA, 2589 bp, Penicillium argentinense
  source 1..2589: mol_type = genomic DNA
atgaagctcgggttgctcgaagccgccgtcttgacggccgcctcggccgccactgcgtcggacctggcatactcgcccccgtactatccgtccccgtggatgaccggcgagggcgactgggccgaggcctaccgccgcgccgtcgacttcgtctccaacctcacactggctgaaaaggtcaacttgaccactggtgcgggctgggagcaagaacgctgcgtcggggagacgggtggtattccccgactgggaatgtggggcatgtgcttgcaagactcgcccctgggcatccgcgacagtgactacaactccggtttcgccgcgggcgtcaatgttgccgccacatgggacaagagactcgcctaccagcgtggtctggcgatgggcgaggagcaccgcgacaagggtgtcgatgtgcagctcggtcctgtggccggcccactgggacgaagccccgatggcggccgtggctgggaagggttctcgcctgatcctgtcctcaccggtgttatgatggcggagactatcaagggtattcaggatgctggtgtcattgcttgcgccaagcactatatcggaaacgagcaggaacacttccgccagtcgggcgaggctcagggctatgggtacaacatcactgagagtgtgagctccaacatcgacgacaagactatgcacgaactgtacctctggccctttgtcgactctatccgcgccggtgtcggctctgtcatgtgctcgtacaatcagatcaacaacagctatggatgccagaacagcgagaccttgaacaggttgctcaaggctgagctgggtttccagggcttcgtcatgtcggattggggtgcgcaccatagcggtgtcagcgctacccttgctggtctggatatgtccatgcccggtgatgtcatcctcggtagcccgtactccttctggggcacgaacctgaccatctccgtgctgaacggtaccgtccccgaatggcgtatcgacgatatggccgtccgcatcatgtcggcctactacaaggtcggccgtgaccgtgtccgtgtccctcccaacttcagctcctggactcgtgatgagtatggctttgagcacttcatggtcagcgagaactacatcaagctcaacgagcgcgtcaatgtccagcgcgaccacgccgcgggtatccgcaagcttggctctgacagcaccgttctgctgaagaacaagggtgctctgcccttgactcacaatgagaagttcgttggtattctgggtgaggatgctggttccaaccctgctggcgctaatggctgtgcggaccgtggttgcgatgacggcacccttgctatgggctggggtagtggtactgctaacttcccttacctgattacccccgagcaagccattcagaacgaggttctgaactatggcaatggccagacaaatgtgtttgccgtgaccaacaacaccaacacggagcagattgctgccattgctgcgcagtcgagcgtcgctctcgtcttcgtaaacgccgactccggcgaaggcttcatcaatgtcgacggcaacgagggtgaccgcaagaacctaaccctctggaagaacggcgaacacctcatcaaaacagctgcggcaaactgcaacaacaccgtcgtcatcatgcacacccccggcgccgtcctaatcagcgactggtacgagcacgacaacatcaccgccatcctctgggccggtctaccgggccaagaaagcggccgcagtatcacagacattctctacgggcgcgtgaacccaggcggcaaaacacccttcacctggggaaagactcgcaaagactacggcgcaccgctcctcacacaacccaacaacggccatggtgcgccgcagcaagacttcacagagggagtcttcatcgactaccgccgcttcgacaaggagcaggaagagcctgtctacgaattcgggtacggactcagctacacgaatttcgaattcagcgatctgcacatcaagtcccttaacgcagacaagtatgtcccgacaacggggacaaccaagcctgcccccgtcttcggcaaaatcggccaggcatccgactatctcttcccgaagggcatccaccgtgtcacccaatacctctacccctacctgaacagcacaaacctcaagtcctcctccggcgacccctactacggcgctaagacagaggagtacatccccgccggcgcaaccaacggctccgcgcaaacccgcctcccatccagcggtgccaacggcggtaatgcgggtctcttcgaggatctgtaccaggttaccgtgacaattaccaacaccggctctgtgcagggcgacgaagttccccagttgtacgttagtctgggcggggagaataaccccgttaaggtcctgcgtgcgtttgacaggatcaccattgcgcctggccagaaggcacagtggactactactcttacccgccgcgatttgtcgagttgggatgttgcaaaacagaattgggtagttactcctgcgcagaagaaggtgtatgttggaaattcgtcgcggagacttccgcttgagtcggagttgcccgcttcgaaatga

SEQ ID NO: 100 - DNA, 2583 bp, Aspergillus wentii
  source 1..2583: mol_type = genomic DNA
atgaggttcggttggttggaggtagccgctcttacggctgcctccgtggtcagtgccaaggatgacctggcattctcccctcccttctacccctctccgtgggcaaacggtgagggtgaatgggcagactcgtacaagcgcgctgtcgagtttgtttcgaacttgaccttggctgagaaggtcaaccttacaactggttctggctggcagcaagagagatgtgttggtgagacgggcgaagtttctagacttggattctggggtatttgtttgcaggattctcccctgggtattcgttttggcgaccactcctccggcttcccctccggcctcaacgttgctgcaacctgggacaaaaagcttgcctacctccgtggtaaggcaatgggcgaggaattccgtgacaagggcattgatgttcaactgggacctgtcgctggtccccttggcgctttccctgatggtggtcgaaactgggagggtttcgctcccgaccctgtgctgactggtttcctgatgtcggagactatcaagggtatccaagatgctggtgttattgctactgctaagcattacatcggtaatgagcaggagcatttccgccaaagcggcgaggctaagggttatggttacaacattactgaaagttcgagctcaaacattgatgacaagacaatgcacgagttgtatctctggccttttgctgatgccgttcgtgctggtgtcggcgcgttcatgtgctcatacaaccagatcaacaacagctatggctgctccaacagctacctgatgaacaaactcctcaagtctgaactaggcttccaaggatttgtcatgagtgattggggtgcacaccacagcggtgttggcgctgctttggctggtttggatatgtccatgcctggtgacactgtcatgggcgacccctacaccttctggggaaccaacctgaccatctcggttctcaacggcaccgtccccgagtggcgtgtggatgacatggccgtccgtatcatggctgcttactacaaggtgggccgcgatgaggttcgcacccctcccaacttcagctcgtggaccaccgaagaattcggatacgcgcattatgctgctcaggaaggatacgagaagaccaactggaatgtcaatgtccgccgcaaccacgccaaggtcatccgcgaaatcggttcggccagcacggttcttctgaagaacaatggcgtgctccccctgaccggtgatgaggactatgtcggaattctgggcgaggacgccggagctaacccctatggtgccaacggctgtgaagaccgtggctgcgacaatggtactctcgctatggcttggggcagtggtagtgcggaattcccttaccttgtgactcccgagcaggccatccagaacgaggttctcaagggcgagggctctgtgttcgctgtgaccgataactgggctctcgagcagatggcttctattgcttcgcagtcctctatctctctcgtctttgtcaatgccgactccggagaaggtttcctcaatgtggacggaaacatgggtgaccgcaagaacttcaccctctggaaaaatggcgagaatgtgatcaagactgtcactgagaactgcaacaacaccgttgtggtcatgcacactgttggcccggtcctgatcaaggactggtatgacaaccctaacatcactgccattgtctgggctggtttgcctggccaggaaagcggaaactctctcgctgatgtgctttacggccgtgtcaaccctagtggcaagagcccattcacctggggcaagactcgcgaggcttatggtccttctctgctcaccacccagaacaacggcaatgacgctccccagcaagatttcacccagggtgtcttcattgactaccgccgatttgacaagttcaacgagactcccatctacgagttcggatacggtctgagttacaccaccttcaactactccaacctcgaagttcgatccctgaatgcatcgcggtacaccccaaccactggcaagactgatgccgcgccttctctgggcgaggctggcaaggcctcggactttttgttccccaagggactgaaccgcatcatcggctacatttatccgtggttgaactcgaccgacctgaagtcggcgtccggagagaagaactatggcatgaaggcttctgagtacattcccgagggagccaccgatggatccgcccaggagctcctgccggccggcggtggccctggtggtaaccctggtctgtatgaagacctcatcgaggtttctgctactattaccaacactggcaaggttgctggtgacgaagtcccacagctgtatgtctcccttggtggtgccgacgaccccgttcttgtcctccgtcagttcgaccgtctccacatcgaggctggaaagcaggcagtgtggaagacgactcttacccgccgtgacctcgccaactgggacgttgcggcgcaagactggaccatcacgaagagcgcgaagaaggtgtacgtgggcagctcttcgcggaacctgcccctcaaggccaagcttcccaccgttcagtag

SEQ ID NO: 101 - protein, 916 aa, Penicillium vulpinum
MSTIIAPVSKELFEYLRTHAFPEICDSFLPHAFFYQQRFLCCLCEILSSIFTAMKFEWLTVAGLTAAANANPLAYSPPYYPSPWMTGAGEWSDAYTRAVDFVSNLTLAEKVNLTTGAGWEQERCVGETGGIPRLGMWGMCMQDSPLGVRLSDYTSGFPSGINVAATWDKRLAYQRGMAMGEEHRDKGVDVQLGPVAGPLGKYPEGGRNWEGFSPDPVLTGVMMAETIKGMQDAGVIACAKHFIGNEQEHFRQSGEAQGYGYNISESVSSNIDDKTMHELYLWPFVDSIRAGVGSIMCSYNQINNSYGCANSYALNKLLKGELGFQGFVMSDWGAHHSGVSSTLAGLDMSMPGDTYLGSPSSFWGANLTISVLNGTVPEWRIDDMAVRIMAAYYKVGRDRFRTPPNFSSWTRDEYSFEHAAVSEGWAKVNERVNVQRDHAQIIRKIGSDSTVLLKNKGGALPLTHGEKFISILGEDAGSNAYGANGCGDRGCDNGTLAMGWGSGTANFPYLITPEQAIQNEVLEYSVGKTSVFAVTDNWALTEMAALASQADVALVFVNADSGEGYINVDGNEGDRKNLTLWKNGEEIIKTASQHCNNTIVVIHSTAAVLISDWYDNDNITAIVWAGLPGQESGRSLVDVLYGRINPGGKTPFTWGKTRKDYGPPLLTVPNNGADAPQDNFEDGVFIDYRRFDKDNTEPIYEFGYGLSYTNFSFSDLKVTPLASSEYNEYKATTGKTKKAPVLGKAGKVSDNLFPEGIKPVRQYLYPWLNSTDLRASSGDPAYGMDSKDYLPEGATDGSPQDLLPSSGASGGNPDLFKNLYQVTATITNTGSVTGDEVPQLYVSLGGDDEPSKVLRQFDRVTIAPGQTLQWTTTLTRRDVSNWDVASQNWVISDAPKKVYVGNSSRKLPLSADLPPI

SEQ ID NO: 102 - protein, 1230 aa, Aspergillus terreus
MKLSILEAAALTAASVVSAQVTFTLPVNLVLDGALTVRKQDDLAYSPPYYPSPWADGHGEWSNAYKRAVDIVSQMTLTEKVNLTTGTGWELERCVGQTGSVPRLGIPSLCLQDSPLGIRMSDYNSAFPAGINVAATWDKTLAYQRGKAMGEEFSDKGIDVQLGPAAGPLGRSPDGGRNWEGFSPDPALTGVLFAETIKGIQDAGVIATAKHYILNEQEHFRQVGEAQGYGFNITETVSSNVDDKTMHELYLWPFADAVRAGVGAVMCSYNQINNSYGCQNSLTLNKLLKAELGFQGFVMSDWSAHHSGVGAALAGLDMSMPGDISFDSGTSFYGTNLTVGVLNGTIPQWRVDDMAVRIMAAYYKVGRDRLWTPPNFSSWTRDEYGFAHFFPSEGAYERVNEFVNVQRDHAQVIRRIGADSVVLLKNDGALPLTGQEKTVGILGEDAGSNPKGANGCSDRGCDKGTLAMAWGSGTANFPYLVTPEQAIQNEVLKGRGNVFAVTDNYDTQQIAAVASQSTVSLVFVNADAGEGFLNVDGNMGDRKNLTLWQNGEEVIKTVTEHCNNTVVVIHSVGPVLIDEWYAHPNVTGILWAGLPGQESGNAIADVLYGRVNPGGKTPFTWGKTRASYGDYLLTEPNNGNGAPQDNFNEGVFIDYRRFDKYNETPIYEFGHGLSYTTFELSGLQVQLINGSSYVPTTGQTSAAQTFGKVEDASSYLYPEGLKRISKFIYPWLNSTDLKASTGDPDYGEPNFEYIPEGATDGSPQPRLPASGGPGGNPGLYEDLFQVSVTVTNTGKVAGDEVPQLYVSLGGPNEPKRVLRKFERLHIAPGQQKVWTTTLNRRDLANWDVVAQDWKITPYAKTIFVGTSSRKLPLAGRLPRVHLVSNQKMKHDVIFFQTPRLLPRQRLNYSQSLRDTAHSHGTTLPSVRPSPETKKTNHQPHGTRRKTEKKNMATTARASSPPGTRPFQPPTAALLVYPATLIIGSLFSVLSPTAQGARASASDDAATAAAPVNYFARKNNIFNVYFVKIGWVWTTLAFAAILLTQPAYTAPSAQRPRRLAQAAARYALATLVWWLTTQWFFGPAIIDRGFVLTGGKCEARAEGTHAASSLPVSPLGMVSAAACKAAGGAWTGGHDVSGHVFMLVLATAMLGFEMGGVFGVEGGKGVGVWSRRFVGAVLGLSWWMLLMTAIWFHTWFEKLTGLLIALGTVYTVYILPRRVVPWRNVVGIPGV

SEQ ID NO: 103 - protein, 882 aa, Stachybotrys microspore
MALASTFLSLYIFLFAYIFTGFACADPDSLYQRQDDGLLTSPPDYPSPWADPEANGWEQAYAQARDFVSQLTLLEKVNLTTGVGWMSERCVGNTGSIPRLGLRGLCLQDGPLGMRFTDYNSAFPAGVVAGATWSRHLWHDRGRMMGEEQYGKGSDVLLGPASGPIGRAPTGGRNWEGFSVDPYHSGVAMAQVVRGIQGAGVIATAKHFVANEQEHFRQAPEAVGYGFNITESLSSNLDDKTLHELYAWPFQDAVRAGVGSIMCSYNQINNSYGCQNSKLLNGILKDEYGFQGFVMSDWQAQHAGAASAAAGLDMSMPGDTVFNTGYSFWGGNLTLGVINGTVPEWRIDDMALRIMAAFFKVGRTVGGQPDINFSSWTRETLGYIHPMAQENLERVNFQVDVRGNHANHIRESAAKGSVILKNNGVLPLSSPKFVVVIGEDAGGNPAGPNGCPDRNCDNGTLAMAWGSGTAQFPYLVTPDQALQRQALEDGTRYESILANNQWTAVQNILREPNTTTIVFADADSGEGFIDVDGNRGDRRNLTLWKDGDALIKNVSSLTSNVIVVLHTVGPVLLTEWYDNPNITAIVWAGVPGQESGNSLTDILYGRRSPGRSPFTWGRTRESYGADLLYEPNNGEGAPQQDFSEGVFIDYRHFDRETAADPENTPIYEFGYGLSWSTFEYSNLQVAKRNVRPYQPTTGNTISAPVFGAPVDPDLSQYTFPPGIRYIYRFIYPYLNTSSSGEDASGDPEYGQEADQFLPPGAIDGSPQPRHPAGGQPGGNPQLWDVLYTVTATITNTGDRMTDEVPQLYISHGGDGEPVRVLRGFDRIERIAPGESVQFRADLTRHDLSNWDVVSQNWVITDDEKTVWVGSSSRNLPLAAPLR

SEQ ID NO: 104 - protein, 857 aa, Rasamsonia emersonii
MRNGLLKVAALAAASAVNGENLAYSPPFYPSPWANGQGDWAEAYQKAVQFVSQLTLAEKVNLTTGTGWEQDRCVGQVGSIPRLGFPGLCMQDSPLGVRDTDYNSAFPAGVNVAATWDRNLAYRRGVAMGEEHRGKGVDVQLGPVAGPLGRSPDAGRNWEGFAPDPVLTGNMMASTIQGIQDAGVIACAKHFILYEQEHFRQGAQDGYDISDSISANADDKTMHELYLWPFADAVRAGVGSVMCSYNQVNNSYACSNSYTMNKLLKSELGFQGFVMTDWGGHHSGVGSALAGLDMSMPGDIAFDSGTSFWGTNLTVAVLNGSIPEWRVDDMAVRIMSAYYKVGRDRYSVPINFDSWTLDTYGPEHYAVGQGQTKINEHVDVRGNHAEIIHEIGAASAVLLKNKGGLPLTGTERFVGVFGKDAGSNPWGVNGCSDRGCDNGTLAMGWGSGTANFPYLVTPEQAIQREVLSRNGTFTGITDNGALAEMAAAASQADTCLVFANADSGEGYITVDGNEGDRKNLTLWQGADQVIHNVSANCNNTVVVLHTVGPVLIDDWYDHPNVTAILWAGLPGQESGNSLVDVLYGRVNPGKTPFTWGRARDDYGAPLIVKPNNGKGAPQQDFTEGIFIDYRRFDKYNITPIYEFGFGLSYTTFEFSQLNVQPINAPPYTPASGFTKAAQSFGQPSNASDNLYPSDIERVPLYIYPWLNSTDLKASANDPDYGLPTEKYVPPNATNGDPQPIDPAGGAPGGNPSLYEPVARVTTIITNTGKVTGDEVPQLYVSLGGPDDAPKVLRGFDRITLAPGQQYLWTTTLTRRDISNWDPVTQNWVVTNYTKTIYVGNSSRNLPLQAPLKPYPGI

SEQ ID NO: 105 - protein, 856 aa, Penicillium waksmanii
MKLGWLEAATLAAASVASASDLAYSPPHYPSPWSTGDGEWAEAYRRAVEFVSNLTLPEKVNLTTGAGWEQEKCVGETGGIPRLGMWGMCMQDSPLGIRDSDYNSGFAAGVNVAATWDKRLAYQRGLAMGEEHRDKGVDVQLGPVAGPLGRSPDGGRVWEGFSPDPVLTGVMMAQTIKGIQDAGVIACAKHFIGNEQEHFRQAGEAQGYGYNISESVSSNIDDKTMHELYLWPFVDSVRAGVGSVMCSYNQINNSYGCSNSYTLNKLLKGELGFQGFVMSDWGAHHSGVGDALAGLDMSMPGDVILGSPYSFWGTNLTISALNGTIPEWRLDDMAVRIMAAYYKVGRDRVRVPPNFSSWTRDEYGYEHFIVSENQIKLNERVNVQRDHASGIRKLGSDSTVLLKNKGALPLTHNERFVAILGEDAGSNPAGANGCSDRGCDDGTLAMGWGSGTANFPYLITPEQAIQNEYLNYGNGQTNVFAVTNNSNTEQIAAMASQATVSLVFVNADSGEGYINVDGNEGDRKNLTLWKNGEQLIKTAAENCNNTIVIMHTPSAVLVGDWYDNENITAILWAGLPVSPIFSMVASTPARRPPFTWGKTRDAYGAPLLTKPNNGQGAPQQDFSEGVFIDYRQFDKADEEPIFEFGFGLSYTKFEFSDVHVTPLKADKYTKTTGRTKPAPVLGKIGEASDYLFPSGIKRVTQYLYPWLNSTNLKESSGDPYYGEKAEKYIPAHARDGSAQQLLPASGPSGGNAGLFEDLFQVTATVTNTGSVVGDEVAQLYVSLGGEGDPVKVLRAFDRITIAPGQNAQWTTTLTRRDLSNWDVASQNWVISDAPKKVYIGNSSRHLPVSIELPSTK

SEQ ID NO: 106 - protein, 801 aa, Aspergillus udagawae
MTLAEKVNLTTGTGIFMGPCAGQTGSALRFGIPNLCLHDSPLGVRNSDHNTAFPAGITVGATFDKDLMYDRGAEMGEEFRGKGINVLLGPSVGPIGRKPRGGRNWEGFGADPSLQAIGGAQTIKGIQSKGVIATIKHYIGNEQEMYRMSNIGQRGYSSNIDDRTLHELYLWPFAEGVRAGVGAVMAAYNDVNSSACSQNSKLLNEILKDELGFQGFVMTDWLGQYGGVSSALAGLDMAMPGDGAVPLLGDAYWGSELSRSILNGSVPVSRLNDMVTRIVATWYKMGQDGDYPLPNFSSNTQDATGPLYPGALFSPSGVVNQYVNVQADHNITARAIARDAITLLKNDDNILPLRKNDSLKIFGADAGPNPDGLNSCADQGCNKGVLTMGWGSGTSRLPYLVTPQQAIANISSNATFYITDSFPSNLAVGSGDIALVFINADSGENYITVEGNPGDRTSAGLNAWHNGDKLVKDAAAKFSKVVVVIHTVGPILMEEWIDLPSVKAVLVAHLPGQEAGWSLTDILFGDSSPSGHLPYTIPRSESDYPSSVGLLSQPLVQIQDTYTEGLYIDYRHFLKANITPRYPFGHGLSYTTFSFSQPTLSVRTAVGSAYPPTRAPKGPTPSYPTTIPNPSEVAWPKNFDRIWRYLYPYLDDPAGAAKNSSKTYPYPTGYTTVPKPAPRAGGAEGGNPALFDVAFAVSVTVTNTGKRPGRAVAQLYVELPNTLGVETPSRQLRQFAKTKVLAPGARETLTMEITRKDISVWDVVVQDWKAPVQGQGVKFWLGESVLDMRAVCEVGGACTII

SEQ ID NO: 107 - protein, 753 aa, Aspergillus transmontanensis
MLLTVDAQRTIIRLSLRGQAMGEEFSDKGIDVQLGPAAGPLGAHPDGGRNWEGFSPDPALTGVLFAETIKGIQDAGVIATAKHYIMNEQEHFRQQPEAAGYGFNVSDSLSSNVDDKTIHELYLWPFADAVRAGVGAVMCSYNQINNSYGCENSETLNKLLKAELGFQGFVMSDWTAHHSGVGAALAGMDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGEGYLSVDGNEGDRNNITLWKNGDNVVKTAANNCNNTVVIIHSVGPVLINEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASQYTPTSGMTEAAKNFGEIGDASEYVYPEGLERIHEFIYPWINSTDLKASSDDSNYGWEDSEYIPEGATDGSAQPRLPASGGAGGNPGLYEDLFRVSVKVKNTGNVAGDEVPQLYVSLGGPNEPKVVLRKFERIHLAPSQEVVWTTTLTRRDLANWDVSAQDWAVTPYPKTIYVGNSSRKLPLQVSLPKAQ

SEQ ID NO: 108 - protein, 694 aa, Penicillium oxalicum
MKLEWLEATVLAAATVASAKDLAYSPPFYPSPWATGEGEWAEAYKKAVDFVSGLTLAEKVNITTGAGWEQERCVGSVMCSYNQINNSYGCSNSYTLNKLLKGELGFQGFVMSDWGAHHSGVGDALAGLDMSMPGDVILGSPYSFWGTNLTVSVLNSTIPEWRLDDMAVRIMAAYYKVGRDRHRTPPNFSSWTRDEYGYEHFIVQENYVKLNERVNVQRDHANVIRKIGSDSIVMLKNNGGLPLTHQERLVAILGEDAGSNAYGANGCSDRGCDNGTLAMGWGSGTANFPYLITPEQAIQNEVLNYGNGDTNVFAVTDNGALSQMAALASTASVALVFVNADSGEGYISVDGNEGDRKNMTLWKNGEELIKTATANCNNTIVIMHTPNAVLVDSWYDNENITAILWAGMPGQESGRSLVDVLYGRTNPGGKTPFTWGKERKDWGSPLLTKPNNGHGAPQDDFTDVLIDYRRFDKDNVEPIFEFGFGLSYTKFEFSDIQVKALNHGEYNATVGKTKPAPSLGKPGNASDHLFPSNINRVRQYLYPYLNSTDLKASANDPDYGMNASAYIPPHATDSDPQDLLPASGPSGGNPGLFEDLIEVTATVTNTGSVTGDEVPQLYVSLGGADDPVKVLRAFDRVTIAPGQKLRWTATLNRRDLSNWDVPSQNWIISDAPKKVWVGNSSRKLPLSADLPKVQ

SEQ ID NO: 109 - protein, 707 aa, Histoplasma mississippiense
MLLPIAFASLAVATEHLTESPPYYPSPWASGQGGWEDAVERARDFVSQLTLVEKVNLTTGVGWMQENCVGQVGSIPRMGLHSLCMQDGPLGIRFADYVSAFPAGVNVGATFSKELAYLRGKAMGEEHRDKGVDVVLGPVVGPLGRSPDGGRNWEGFSPDPVNSGLLVAETIKGIQSASVIACVKHFIGNEQERFRQGPEAQGYGFDISESSSSNIDDVTMHELYLWPFADAVRAGVGSVMCSYNQINNSYGCGNSYTQNKLLKAELGFQGFIMSDWQAHHSGVGSALAGLDMSMPGDTVFGTGRSYWGPNLTIAVANGTIPEWRVDDMAVRIMAAYFKVGREAAKVPVNFNSWTRDEYDYTHALVKEGYGKVNERINVRAKHASIIRQVGAASVVLLKHTGSLPLTGLEKNAAVIGEDAGPNLWGPNGCPDRRCDNGTLAMGWGSGTADFPYLVTPAEAIQNEILSKGEGSVFPIFDNWASDQIKSAASQATVSLVFVNADSGEGFISVDGNEGDRKNLTLWKGGDELIQTVASYCNNTVVVIHSTGPVLVGEWNEHPNITAILWAGLPGQESGNSIADVLYGKVNPGGRSPFTWGRTAEDYGASILKEPNEGNGAPQVDFTEGIFTDYRAFDKADIKPIYEFGFGLSYTSFSYSDLNVEVPPWTLITDYRTKITSLLAQPMALHKNFFLLVGDPVEILVFMKYYIV

SEQ ID NO: 110 - protein, 777 aa, Exophiala dermatitidis
MHELYLWPFSDAVRAGTGSVMCSYNQINNSYGCANSYTMNHLLKNELDFQGFIMSDWQAQHSGVGTALAGLDMTMPGDTLFNTGRSYWGTNLTIAVINGTVPEWRVDDMATRIMAAYYYVGRDTHYTPTNFYAWSRNTYDKIHQVDPQSPIGLVNEHINVQDRHRDIVRQVGQASNVLLKNTGGLPLTGKERQVGIFGYDAGSNPWGANGCSNRGCDNGTLAMGYGSGTAEFPYLVTPEQAITQHVLTQTDGEVFAILDNYADAQIKSLASTADVALVFANAQSGEGFITIDGNTGDRNNLSLWLGADRLIHNVTKYNKNVIVVMHTVGPVNVSAWYDNENVTGIIWAGLPGQESGNAIVDALYGLINPGGKLPFTIGRNREDYGTDILYEPNNGQFNAPQSLFSEGVFIDYRHFDQYNIEPIYEFGFGLSYTTFEYSNLVITPGNPAPYTPTSGQTEPAPVLGNASTDPSQYVFPNGTITYRPYLYIYPYLNSTDLRASSDDTDYGLPTDQYVPPGATDGSPQPLLPAGGAPGGNAGLYEVVATVSATITNTGSVEGDEVAQLYVSLGEGEPPKVLRGFDRLTIAPGASTTFTANLTRRDVSVWDTVSQNWVQVSNPTIYVGTSSRKLPLSGVLSSSGGGSGAQSSSSASGGSGGGSSSGWGYGQSSTASGSAPAPVSQLSDGQPQVPTGRPVTQVSDGQPQAPTGNPVSQISDGQPQAPAHTGGAPVSQISDGQPQVPTGTGPAVTQISDGQPQNPTGGPAVSQLSDGQPRATTA

SEQ ID NO: 111 - protein, 860 aa, Aspergillus welwitschiae
MRFTLIEAVALTAVSLASADELAYSPPYYPSPWANGQGDWAEAYQRAVDIVSQMTLAEKVNLTTGTGWELELCVGQTGGVPRLGIPGMCAQDSPLGVRDSDYNSAFPAGVNVAATWDKNLAYLRGQAMGQEFSDKGADIQLGPAAGPLGRSPDGGRNWEGFSPDPALSGVLFAETIKGIQDAGVVATAKHYIAYEQEHFRQAPEAQGYGFNITESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGFKYYYVSEGPYEKVNQFVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVIIHSVGPVLVNEWYDNPNVTAILWGGLPGQESGNSLADVLYGRVNPGAKSPFTWGKTREAYQDYLYTEPNNGNGAPQEDFVEGVFIDYRGFDKRNETPIYEFGYGLSYTTFNYSNLQVEVLSAPAYEPASGETEAAPTFGEVGNASDYLYPDGLQRITKFIYPWLNSTDLEASSGDASYGQDASDYLPEGATDGSAQPILPAGGGAGGNPRLYDELIRVSVTIKNTGKVAGDEVPQLYVSLGGPNEPKIVLRQFERITLQPSEETQWSTTLTRRDLANWNVETQDWEITSYPKMVFVGSSSRKLPLRASLPTVH

SEQ ID NO: 112 - protein, 860 aa, Aspergillus eucalypticola
MRFTLIEAVALTAVSLASADELAYSPPYYPSPWANGQGDWAQAYQRAVDIVSQMTLAEKVNLTTGTGWELELCVGQTGGVPRLGVPGMCLQDSPLGVRDSDYNSAFPAGMNVAATWDKNLAYLRGKAMGQEFSDKGADIQLGPAAGPLGRSPDGGRNWEGFSPDPALSGVLFAETIKGIQDAGVVATAKHYIAYEQEHFRQAPEAQGYGFNISESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTVSVLNGTVPQWRVDDMAVRIMAAYYKVGRDRLWTPPNFSSWTRDEYGYKYYYVSEGPYEKVNQYVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVVIHSVGPVLVNEWYDNPNVTAILWGGLPGQESGNSLADVLYGRVNPGAKSPFTWGKTREAYQDYLVTEPNNGNGAPQEDFVEGVFIDYRGFDKRNETPIYEFGYGLSYTTFNYSNLEVQVLSAPAYEPASGETEAAQTFGEVGNASDYLYPDGLQRITKFIYPWLNSTDLEASAGDSSYGQDSSDYLPEGATDGSAQPILPAGGGPGGNPRLYDELIRVSVTIKNTGKVAGDEVPQLYVSLGGPNEPKIVLRQFERITLQPSEETTWNTTLTRRDLANWNVEKQDWEITSYPKMVFVGSSSRKLPLRASLPTVH

SEQ ID NO: 113 - protein, 838 aa, Aspergillus luchuensis
MRFTLIEAVALTAVSLASADELAYSPPYYPSPWANGQGDWAQAYQRAVDIVSQMTLAEKVNLTTGTGYVLVRLAGFPADYNSAFPSGMNVAATWDKNLAYLRGKAMGQEFSDKGADIQLGPAAGPLGRSPDGGRNWEGFSPDPALSGVLFAETIKGIQDAGVVATAKHYIAYEQEHFRQAPEAQGYGFNISESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTVSVLNGTVPQWRVDDMAVRIMAAYYKVGRDRLWTPPNFSSWTRDEYGYKYYYVSEGPYEKVNHYVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVIIHSVGPVLVNEWYDNPNVTAILWGGLPGQESGNSLADVLYGRVNPGAKSPFTWGKTREAYQDYLVTEPNNGNGAPQEDFVEGVFIDYRGFDKRNETPIYEFGYGLSYTTFNYSNLEVQVLSAPAYEPASGETEAAPTFGEVGNASNYLYPDGLQKITKFIYPWLNSTDLEASSGDASYGQDSSDYLPEGATDGSAQPILPAGGGPGGNPRLYDELIRVSVTIKNTGKVAGDEVPQLYVSLGGPNEPKIVLRQFERITLQPSEETKWSTTLTRRDLANWNVEKQDWEITSYPKMVFVGSSSRKLPLRASLPTVH

SEQ ID NO: 114 - protein, 860 aa, Aspergillus heteromorphus
MRFGWIEAAALTAASVVSADELAYSPPFYPSPWANGQGDWASAYERAVAIVSQMTLAEKVNLTTGTGWELEKCVGQTGGVPRLGIPGMCGQDSPLGVRDSDYNSAFPAGINIGATWDKNLAYLRGQAMGQEFSDKGADFQLGPVAGPLGRAPDGGRNWEGFSPDPALTGVLFAETIKGIQDAGVVATAKHFIGYEQEHFREAPEAQGYGYNITESGSANIDDKTMHELYLWPFADAVRAGVGAIMCSYNQINNSYACQNSYALNKLLKGELGFQGFVMSDWEAHHSGASAAMAGLDMSMPGDVVFDSGTSYWGTNLTIGVLNGTVPQWRVDDMAVRIMAAYYKVGRDRLWTPPNFSSWTRDEYGFKYYYSSEGPYEVVNHFVDVRRNHSDLIRRIGADSTVLLKNDGCALPLTGTERKVALIGEDAGSNPYGADGCSDRGCDNGTLAMGWGSGTTEYPYLVTPEQAIQAEVIQNGGTVFAVTDNWAISQMETLAAEATVSLVFVNADSGEGYIDVDGNMGDRNNLTLWGNGDNVIKAAASNCNNTIVIIHSVGPVLVNEWYDHPNVTAILWAGLPGQESGNSLADVLYGRVNPGAKSPFTWGKTRESYQDYLITEPNNGDGAPQEDFTEGVFIDYRGFDKRNETPIYEFGYGLSYTTFNYSQLQVQALNASAYTPASGETEAAPTLGEAGNASDYLYPSGLQRITAFIYPWLNSTDLEASSGDSSYGQSSDFLPEGATDGSAQPILPAGGGPGGNPALYDDLIQVSVTIKNTGDIAGDEIPQLYVSLGGPDEPRIVLRKFDRITLQPSEEYEWTTTLTRRDLSNWDVAAQDWIVTSYPKKVYVGSSSRKLPLRASLPTVQ

SEQ ID NO: 115 - protein, 860 aa, Aspergillus aculeatus
MKLSWLEAAALTAASVVSADELAFSPPFYPSPWANGQGEWAEAYQRAVAIVSQMTLDEKVNLTTGTGWELEKCVGQTGGVPRLNIGGMCLQDSPLGIRDSDYNSAFPAGVNVAATWDKNLAYLRGQAMGQEFSDKGIDVQLGPAAGPLGRSPDGGRNWEGFSPDPALTGVLFAETIKGIQDAGVVATAKHYILNEQEHFRQVAEAAGYGFNISDTISSNVDDKTIHEMYLWPFADAVRAGVGAIMCSYNQINNSYGCQNSYTLNKLLKAELGFQGFVMSDWGAHHSGVGSALAGLDMSMPGDITFDSATSFWGTNLTIAVLNGTVPQWRVDDMAVRIMAAYYKVGRDRLYQPPNFSSWTRDEYGFKYFYPQEGPYEKVNHFVNVQRNHSEVIRKLGADSTVLLKNNNALPLTGKERKVAILGEDAGSNSYGANGCSDRGCDNGTLAMAWGSGTAEFPYLVTPEQAIQAEVLKHKGSVYAITDNWALSQVETLAKQASVSLVFVNSDAGEGYISVDGNEGDRNNLTLWKNGDNLIKAAANNCNNTIVVIHSVGPVLVDEWYDHPNVTAILWAGLPGQESGNSLADVLYGRVNPGAKSPFTWGKTREAYGDYLVRELNNGNGAPQDDFSEGVFIDYRGFDKRNETPIYEFGHGLSYTTFNYSGLHIQVLNASSNAQVATETGAAPTFGQVGNASDYVYPEGLTRISKFIYPWLNSTDLKASSGDPYYGVDTAEHVPEGATDGSPQPVLPAGGGSGGNPRLYDELIRVSVTVKNTGRVAGDAVPQLYVSLGGPNEPKVVLRKFDRLTLKPSEETVWTTTLTRRDLSNWDVAAQDWVITSYPKKVHVGSSSRQLPLHAALPKVQ

SEQ ID NO: 116 - protein, 861 aa, Aspergillus caelatus
MKLGWFEVAALAAASVVSAQDDLAYSPPFYPSPWADGQGEWAEVYKRAVEIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWEGFSPDPALTGVLFAETIKGIQDAGVIATAKHYILNEQEHFRQQPEAAGYGFNVSDSLSSNVDDKTIHELYLWPFADAVRAGVGAVMCSYNQINNSYGCENSETLNKLLKAELGFQGFVMSDWTAHHSGVGAALAGMDMSMPGDVTFDSGTSFWGANLTIGVLNGTIPQWRVDDMAVRIMAAYYKVGRDTKYTPPNFSSWTRDEYGFAHNHVSEGAYERVNEFVDVQRDHADLIRRIGAESTVLLKNKGALPLSRKEKLVALLGEDAGSNPWGANGCDDRGCDNGTLAMAWGSGTANFPYLVTPEQAIQNEVLQGRGNVFAVTDSWALDKIAAAARQASVSLVFVNSDSGEGFLSVDGNEGDRNNITLWKNGDNVVKTAANNCNNTVVIIHSVGPVLINEWYDHPNVTGILWAGLPGQESGNSIADVLYGRVNPGAKSPFTWGKTRESYGSPLVNEANNGNGAPQSDFTQGVFIDYRHFDKFNETPIYEFGYGLSYTTFELSDLHVQPLNASQYTPTSGMTEAAKNFGETGDASEYVYPEGLERIHEFIYPWINSTDLKASSDDSNYGWEDSKYIPEGATDGSAQPRLPASGGAGGNPGLYEDLFRVSVKVKNTGKVAGDEVPQLYVSLGGPNEPKVVLRKFERIHLAPSQEVVWTTTLTRRDLANWDVSAQDWTVTPYPKTIYVGNSSRKLPLQASLPKAR

SEQ ID NO: 117 - protein, 854 aa, Madurella mycetomatis
MANLVLALLLFLSALTSAATDTPFLGYPSPWATNITDPTWAAAYAQAIAFVANLTLTEKVNLTTGTGWEADRCIGATGGIPRLGFRPFCLMDGPLGVRYTDHNSAFPAGVNTAATFSRRLMRLRGEAMGAEFRGKGIDVMLGPVAGALGRVPQGGRNWEGFSPDPYLTGVAMAETIQGIQSRGVVACAKHYILNEQEHFRGSIDVRIDDRTMHELYLWPFADAVRAGVGSVMCSYNKINGTYACENEWTTNYLLKNELGFQGFVLSDWGAQHNTLGSALGGLDMAMPGDGGPPPYRAWWGGALTEAVLRGDVPQWRLDDMAVRIMAAYFRVHTGNYTSRPDINFSAWTNSTVGPLYPAANQSYTVVNEFVDVQSDHASLIREIGAKSVVLLKNAYDLLPLRQPGPPIIAVIGDDAQDHPLGPNACPERGCLNGTLAMGYGSGTANFPYLVSPLTALTEQARADNTTLLYAPSNWDLDAAITTARNASIAIVFAAATSGENFITVDGNAGDRNNLTLWANGDALIKAVASINPNTIVVLHTPGPVILDYAEEHPNISAILWAGLPGQESGNALVDVLYGKVNPQGRSPFTWGDSVEEYGAQLMFEAENPRAPVQSFDEGVFIDYRKFSTFGGKLTYPFGFGLSYTKFRYSGLSVVRKEEVEGRFEPAEGLTGPAPTFGVVEAELGVHTAPEGFTRISPYVYPWLNNSESLVTGNSTGASEFPAAARNGSAQPVLPASGAPGGNPGLYEVLYTITASIENVGEVAGTEIPQLYVQLGGEENPFGVLRGFDEVELEPGETKNVTFELTRRDVSNWNTSTQNWEITDREKVVFVGSSVRDIRLNASLPAPVGMAWRHG

SEQ ID NO: 118 - protein, 975 aa, Aureobasidium pullulans
MSQSQDYELTPRESPRQSLDHPEDYRLSTSSRDSISSSINELDPLKTPNRPYKDDVSQTHDSVFVTRRRSTWRRYLLPSRMCCMMVLLFTAALVMLLSAGGIWVYKTEPEGGQSDPWYPAPRGGSVTAWEEAYKKAAALVGQMSVVEKVNITTGTGWEMGMCVGNTGPVERLGFPSLCLQDGPLGLRFADNITAFPAGITLGTTWNKELIYLKGRAHGREARGKGVHIALGPSMGPFGRLPAGGRNWEGFGSDPVLQGLAAAAMIRGIQEEGIIATAKHYIANEQEHFRQSWEWGTPNAISSNLDDRTLHEIYAWPFAESVKAGVGSVMCSYNLVNQSYACQNSKLLNGILKDELGFQGFIQSDWLAQRSGVASALAGLDMSMPGDGLRWMDGQSVWGGELTKAVFNGSVPMERLNDMVLRVVAAWYQFGQDDKKKWPAEKDGGGPNFSSWTNDEIGLLHPGSNDTTKGVVNKFVNVQGEGDDSHGKLARQIAAEGTVLLKNDDHVLPLNRDGKKLTKNGDKLRVAIFGEDAYNNGKGPNACADRACNEGTLAQGWGSGTVEFPYLVAPAEGIHLGFDVESVTLSDYPSNYEDLDKHPSRAADQDLCFVFINSDGGEGYVAWKDVKGDRNDLYAQKGGDELVQKVAAKCGGPTIVVVHAVGPVILEKWIDLPGVKAVLYANLPGQESGNALASIVFGDINPSGRLPYTIAKNEDDYGPTSKILYRPNAVIPQQNFSEGLYVDYRYFDKHDIEPRYEFGFGLSYTTFHLSSLFIHPLATEYSTLPAKRPESITPPHLDVSIPDAREALWPSNFIALKKYIYPYINSVDDIKTGKYPYPVGYEIAQPLSQAGGAEGGNPDLYTPLAVVQASLTNTGSLPGDCVVQLYISYPSTPIIDSLGNTVDFPVRVLRAFDKLFVTPEKRVEVSLQLTRKDLSFWDVGLQNWVLPMGDFEVQLGFSSRDIQQKGDILSFSRLLL

SEQ ID NO: 119 - protein, 1220 aa, Fusarium oxysporum
MALIICFYYPISLGEAPDSDHTLKEKILGLGLKSAVILAGALVCLFLALQWGGTKHPWSDSRVWGCLVGFGLLLTLFVYIQIRQGEAALIRPRIISQRSVFLGCLFSALYQGAMTTQSYHLPFYFQAVKGVDPQSSGVDILPHGVTVTIATLITGSIITWLGYYVPFMWAGSAIFTIGAGLLYTISQNTPLARWFGYEVLAGAGFGIAIQIPIFAVQVVLVAGDIPLGTVLIILSQALGGSVGLSISQNVFQNSLRQRLNTIADIDIQVVIAAGGTDLEHVVSADSLAHVRDAFRYGISNAFLVSTALGGVAVLASIGMERKKIKSKKEGVDLYKTARPFDVSVFSSRGSIDTKMTLAKVAFTLANLVAVGSSAKNVADGFVAAPYYPAPYGGWDEAWADSYARAKKMVDSMTLAEKTNITAGTGLFMADRVGFPQLCLNDAANGVRLADNVTVFPDGITAGATFDKKLMYERGVAIGKEARGKGVNVWLGPSVGPIGRKPKGGRNWEGFGADPSLQGIGARETIKGVQEQGVIATVKHLIGNEQEMYRRQTTDVVSPAYSANIDDRTMHELYLWPFAEAVKVGVGATMTAYNRVNGTISSEHSYLINALLKEELGFQGFVMTDWLSQITGVQSAIAGMDMSMPGDTIIPLFGNSLWMYELTRSALNGSVPMSRLNDMATRIVATWYQFEQDKDFPSVNFDTNTYNRVGPLYPAAWPNSPSGVVNQYVQVQDDHDEIARQIAQDAITLLKNDEKLLPLSTKSSLKVFGTGAQTNPDGANACVDRSCNKGTLGQGWGSGTVDYMYLDDPIGAIKKAAGDVTFYNTDKFPSVPSPSDDDVAIVFVTSDSGENQYTVEGNNGDRNADKLNVWHNGDALIKAAAAKYKNVVVVIHTVGPVLVDQWIDLPSVKSVLVAHLPGQEAGKSLTNILFGDASPCGHLPYSITKKEDDMPESVTKLIDSGFIDAPQDTYSEGLFIDYRWLNKEKIQPRYAFGHGLSYTNFTYTNATIKRGTQLSQYPPKRPAKGKVLDYSQDIPDYKEAIKPSSFKTVWRYLYSWLSESDAKSAAAKAETSKYPYPDGYSTAQKTALPKAGGVSGGNPALWDEAYTLSVVVTNAGSKFSGKASVQAYVQFPSVAGYETPVIQLRDFEKTKVLEPGSSETVQLTLTRKDLSVWDVKAQDWLVLDGEFKIWLGSASDKLDAVCFTDDLGCEHDVKGPVSYDS

SEQ ID NO: 120 - DNA, 1437 bp, Cellulomonas wangleii
  source 1..1437: mol_type = genomic DNA
atgacccccccaccccagctccgacgcgggcgccccctggtcgcacgcggcgcctccgccatcgcgatcgtcgcgctcgcggtgacggccgccgcggccatcccggcgcaggccgccggctcgacgctccaggaggccgccgcccagagcggccgctacttcggcaccgcgatcgccgcgaacaagctcagcgactcgacctacgtgaccatcgcgaaccgtgagttcaacatgatcacggccgagaacgagatgaagatggacgcgacggagccgaaccagaaccagttcaactacagccagggcgaccggatcctcaactgggccaagcagaacggcaagcaggtccgcgggcacgcgctcgcgtggtactcccagcagccggagtggatgaagcgcatggagggctccgcgctgcgcagcgcgatggtcaaccacgtcacccaggtggcgacgcactacaagggtcagatctacgcctgggacgtcgtgaacgaggcgttcgccgacggcagctccggcggacgccgcgactccaacctggagcgcaccggcagcgactggatcgaggtggccttccgcgccgcgcgcgccgccgacccggccgcgatcctctgctacaacgactacaacatcgacgactggtcgcacgccaagacccagggcgtctaccggatggtccgtgacttcaagcagcgcggcgtcccgatcgactgcgtcggcctgcagtcgcacttcaacccgcagagcccggtcccggccaactaccagacgacgatcgagagcttcgccgccctcggcgtcgacgtgcagatcaccgagctcgacatcgagggctccggctcggcgcaggccgagaacttccgcaaggtgacccaggcctgcctcaacgtcgcccgctgcaccggcatcacggtgtggggcgtgcgtgacacggactcgtggcgcgcctcgggcaccccgctgctgttcgacggcaacggcaacaagaagcaggcgtacacctcggtgctcaacacgctgaacgcggcgagcccgaccacgccgccgcccaccacgccgccgccgacgaccccgccgccggtgacgcccccgcccacgaccccgccgcccaccacgccgccgccgacgaccccgccgccgacgaccccgccgccggtgacgggcagctgctccgccgcactgaccatcgccaacgcgtggcccggtggctaccagggcacggtcacggtgacggccggctcgtcgtccctcaacgggtggcgggtgaccctgccgggtggcgtctccacgaacaacgtgtggaacggcgtggtctccggcaacgtcgtgagcaacgcgccctacaacggctccgtcggggccgggcagtcgacgaccttcgggttcatcgggaacggcaacgccccgtcgccgacctccctcacctgctcctga

SEQ ID NO: 121 - DNA, 1419 bp, Cellulomonas shaoxiangyii
  source 1..1419: mol_type = genomic DNA
atgctccgacgagggagcaggcgcggccccggcgctcgaggcgcagccgtcctcgccgccctgaccctcgcggcgaccgccgcggccgccatcccggctcaggccgccggctcgacgctgcaggacgcggccaacgaccgcggccgctacttcggcacggcgatcgccgcgaaccgcctcagcgactcgacctactcgaccatcgcgaaccgtgagttcgacatgatcacggccgagaacgagatgaagatggatgcgacggagccgtcgcagaaccagttcaactacaccaacggcgaccgcatcgtgaactgggcgctccagaacggcaagcaggtccgcgggcacgcgctggcgtggcactcgcagcagccgggctggatgcagaacatgtccggcacggcgctgcgcaacgccatgctcaaccacgtcacgcaggtcgccacgcactaccgcggcaagatctacgcctgggacgtcgtgaacgaggccttcgccgacggctcgtccggcgcccgccgcgactccaacctgcagcgcaccggcaacgactggatcgaggcggccttccgcgccgcccgggccgccgaccccggcgcgaagctctgctacaacgactacaacaccgacgactggacgcacgcgaagacgcaggccgtgtacaacatggtccgcgacttcaaggcgcgcggcgtcccgatcgactgcgtcggcctgcagtcgcacttcaacgcgcagagcccggtgccgagcaactaccagacgaccctgtcgagcttcgccgcgctcggcgtggacgtgcagatcaccgagctcgacatcgagggctcgggctccgcgcaggcggagagctaccggcgcgtcgtccaggcgtgcctcaacgtctcccgctgcaccggcatcacggtgtggggcgtgcgggacaccgactcgtggcgcgcctcgggcacgccgctgctcttcgacggccagggcaacaagaaggcggcgtacaccgcggtcctcgacacgctgaacagcggacccgggaccaacccgacgacgcctccgcccaccagccccccgccgacgaccccgccgccggtcaccccgccgccgacgacgcccccgcccacgaccccgccgccggtcaccccgccgcccagcggcacctgctccgcggcactgacgatcgcgaacagctggggcggcggctaccaggccaccgtcacggtgcgggccggttcgtccgctctcaacggatggcgggtcaccctgcccggcggcgtcacgacgtcgaacctgtggaacggcgtcctcgccggcagcgtcgtgacgaacgcgccgtacaacggctccgtcggagccggccagtcgacgtccttcgggttcatcggcaacggcagcgcacccgccgcgggagcgctgagctgcagctga

SEQ ID NO: 122 - DNA, 1446 bp, Micromonospora sp.
  source 1..1446: mol_type = genomic DNA
atggacaaggtgctcgcccgcggcagcgggagcccgaccgcccgataccggccgcgtgccgccctgctgtcggccgccgtcggcgcggcgctggtcgcggccacggtcgcgatggcgaccagcgccagcgccgggaccaccctgggcgcggcggcggcggaacagggccgctacttcggcaccgcggtggccgcgaacaagctgtcggacgccacgtacgtcggcatcctgaaccgcgagttcactatggtcacccccgagaacgagatgaagtgggacgccaccgagccgtcccagggccagttcagctacaccaacgccgaccggatcgtcgcccacgcccaggccaacggcatgcgggtacgcggccacgcgctggcctggcactcgcaacagcccggctgggcgcagaacctgtccggcagcgcgctgcgccaggcgatggtcaaccacatcacccaggtcgccacccactacaagggcaagatctacgcctgggacgtggtcaacgaggcgttcgacgacggcagcggcggccggcgcgactccaacctccagcgcaccggcaacgactggatcgaggtggcgttccgcaccgcgcgcgccgccgacccgggcgccaagctctgctacaacgactacaacaccgacaactggacctgggccaagacccagggcgtctacaacctggtcaaggacttcaaggcccggggcgtgccgatcgactgcgtcggcctgcagtcgcacttcaacagcggctcgccgtacccgagcaactaccgcaccacgttgcagaacttcgccgccctcggcgtcgacgtgcagatcaccgaactcgacatcgagggttcgggcagcgcccaggccaccacgtacgccaacgtggtcaaggactgcctggccgtgtcgcggtgcaccggcatcaccgtctggggaatccgggacagcgactcctggcgggccggcggcaccccgctgctcttcgacggcaacggcaacaagaaggcggcctacaccgccgcgctcgacgcgctcaacgccggcggcacgccgccgccgaccaccacgccgccgaccaccacgcccccgaccacgactccgccgacgaccacgccgcccaccaccgcgccgcccacgactcccccgcccaccggcggcgggtgcaccgcgtcgctcaccaccaaccagtggcccggcgggttcgtcaccaccgtgcgcgtcaccgccggcgccggcgcgctcaacggctggacggtgacgctcaccgtgccgtccggctcggcggtcaccaacacgtggagcgcccaggccagcggcgccagtggcgcggtgacgttccgcaacgtcgcctacaacggccaggtcggtgccggcggcagcaccgagttcggcttccaggggaccggcacggccccctccggcacgcccacctgcgcggcgggctga

SEQ ID NO: 123 - DNA, 1425 bp, Xylanimonas cellulosilytica
  source 1..1425: mol_type = genomic DNA
atgaaccatcagcaccaccgtcgtaggttgcgcagcgtcgcagccgtcgcgctcgcctccctggtcgtcgccacgggagtggtgaccgcccaggcggccggatccacgctccaggaagcggcgggcagccgctacttcggcacggccatcgcggccaacaagctctcggactcgacctactcgaccatcgcgaaccgtgagttcgacatgatcacggccgagaacgagatgaagatggacgccaccgagccgtctcagggcagcttcaacttcaccaacgccgacaggatcgtcgactgggccaccgccaacggcaagcggatgcgcggtcacgccctcgcgtggcactcgcagcagccggggtggatgcagaacatgtcgggcaccgcgctgcgcacggcgatgctcaaccacgtcaccgaggtcgctgcgcactacaagggcaagatctacgcctgggacgtcgtcaacgaggccttcgccgacgggtcctcgggcgcccgccgcgactcgaacctgcagcgcacgggcgacgactggatcgaggccgcgttcagggccgcccgggccgccgacccgtccgccaagctctgttacaacgactacaacacggacaactggaactgggagaagacccaggccgtgtacgccatggtcaaggacttcaaggaacgcggcgtgccgatcgactgcgtcgggctccagtcccacttcaactcgggcagcccctacccgagcaactaccgcacgacgctgcagaacttcgcggcgctcggcgtcgacgtccagatcaccgagctggacatcgagggctcgggcagcacgcaggccgacacgtacgccaaggtggtcgccgactgcctcgcggtgagccgctgcaccggcatcacggtgtggggcgtgcgcgactccgactcgtggcgcgcgagcggtacgccgctgctgttcgacgcgtcgggcaacaagaaggccgcctacacgtcggtgctgaacaccctcaacggcggtgcgacgccgacgccgacgccgacgccgacgccgacgccgaccccgactcccacgccgactccgaccccgacgccgacgccgactcccacgccgacgccgacgccgacgccgactcccacgccgacgccgacgtccggtccgtgcaccgccacgatgacgatcacgaactcgtggcagggcggcttccagggtgaggtcacggtcaaggcgggcagtgcccgcacgtcctggtcgacgtcgttcacctcggccgccacggtccaagtctggaacggggtgcactcgacctccggctcggtgcacacggtgtcgaaccagccgtacaacggcacgctggccgccggtgcgtcgacgacgtacggcttcacggccaccgggacggcgccgagcagcccgcccgcggtgacctgctcgtga

SEQ ID NO: 124 - DNA, 1443 bp, Verrucosispora sp.
  source 1..1443: mol_type = genomic DNA
atgaacaacgcccacgcccgcgccagcgggcggccggctacccgactgcgaccgcgcgccgcgctggtctcggccgcggtcggtctcgccctcgtcgccaccagcatggtggtgacctccagcgcccaggccgccgagtccaccctcggtgcggccgcggcccagtccggccggtacttcggtgccgcggtggcggcgaacaagctgtccgactccacctacgtcggcatcctgaaccgcgagttcaactcggtcacggccgagaacgagatgaagatcgacgcgctcgagccgcagcagaacaacttcacgttcggcaacgcggaccggatcgtcaaccacgccctgtcccggggctggaaggtccgggggcacaccctggcctggcactcgcagcagcctggctggatgcagcagatggaaggcacggcgctgcgtaacgcgatgctgaaccacgtcacccgggtcgcgacctactaccgcggcaagatcgactcgtgggacgtggtgaacgaggcgttcgacgacggcaacaacggcgcgcgccgtaactcgaacctgcagcgcaccggtaacgactggatcgaggccgcgttccgggccgcccgggccgccgacccgggcgccaagctctgctacaacgactacaacaccgacaactggacctgggccaagactcaggccgtctaccggatggtgcaggacttcaagcagcgtggcgtgccgatcgactgcgtcggcctccagtcgcacttcaacagcggctcgccgtatccgagcaactaccgcaccacgctgtccagcttcgccgcgctcggcgtggacgtgcagatcaccgagctggacatcgaaggctccggcagcacccaggccaacacctaccgcaacgtggtcaacgactgcctcgcggtgccccgctgcaacggcatcaccgtgtggggaatccgggacaccgactcctggcgttccggtggcaccccgctgctcttcgacggcaacggcaacaagaagcaggcgtacgacgcgacgctccaggcgctgaactcgggcggcaccaccccgccgcccacgaccccgccgccgcccacgaccccgccgcccaccacgccgccgcccacgaccccgccgccgaccaccccgccgcccggcgggtccggctgcacggcaacggtgtccctgaaccagtggaacggtggtttcgtcgccacgatccgcgtcaccgccggttccgcgagactgaacggctggtcggtgggcatcgggattccgggcggtagctcggtgaccaacacctggaacgcccaggccagcggcaacagcggcaacgtgacgttccgcaacgtcagctacaacggcaccgtgaacgccggtgccaccacggagttcggcttccagggcaccggtaccggtccatccggtacccccacctgcaccggtagctga

SEQ ID NO: 125 - DNA, 2043 bp, Saccharothrix syringae
  source 1..2043: mol_type = genomic DNA
atgtccagaactgctgtcacagctgccggcaggtcggaggcacgaggccggtcgcggcgcgccgcggtggtggtcggtgcgatcgggttgctgagcgcggcggccgtggtgctgccgaacgtggccaccgccggcaccacgctgggcgcgtccgccgcggagagcgggcgctacttcggcacggcggtcgcggccaacaagctctcggactcgacctacgtgggcatcctcaaccgtgagttcgacatggtcacggctgagaacgagatgaagatggacgccacggagccgaaccagaaccagttctcgttcggcaacggtgaccggatcgtcaaccacgcccgcaaccagggcaagcgggtccgtggccacgcgttggcgtggcactcgcagcagccgggctggatgcagaacatgtccggcacggcgttgcgcaacgcgatgctcaaccacgtcacccaggtcgccacgtactacaagggcaagatctacgcctgggacgtggtcaacgaggcgtacgccgacggcagctccggcggtcggcgtgactcgaacctccagcggaccggcaacgactggatcgaggccgccttccgggccgcccgcgccgccgacccgaacgcgaagctgtgctacaacgactacaacaccgacaactggtcgcacgccaagacccagggcgtgtaccggatggtgcaggacttcaagtcgcgcggtgtgccgatcgactgcgtcggcttccaggcgcacttcaacagcggcaacccggtgccgtcgaactaccacaccaccctgcagaacttcgccgacctcggcgtggacgtccagatcaccgagctggacatcgagggctccggcagcacccaggcccagcagtaccagggcgtggtccaggcgtgcctcgcggtgacccgctgcaccggcatcacggtgtggggcatccgcgacagcgactcgtggcgttcctcgggcaccccgctgctgttcgacggctcgggcaacaagaaggccgcctacacctcggtcctcaacgccctcaacgcgggcagcaccgcgccgccgtccaccaccacgacgacgaccccgccgaacacctcggactgccgcgccggctacgtcggcctgacctacgacgacggccccaacggcagcaccaccacgcagctgctcaacgcgctgcggtcggccggcctgcgcgccacgttcttcaaccagggcaaccgggtccagcagaaccccggcctggccaaggcgcagcgcgacgccggcatgtgggtcggcaaccacagctggagccacccgcacatgacccagctgagccagtcgcagatggcctcggagatctcccagacccagcaggccatccagtcggccaccggtgaggcgccgaagctgttccgcccgccctacggcgagaccaacagcacgctcaagtcggtcgaggcccagtacggcctgaccgaggtgctgtggagcgtcgactcgcaggactggaacaacgccagcaccgcgcagatcgtgcaggccgcgtcgaccctgcagaacggcggcgtgatcctgatgcacgacggctaccagacgacgatcaacgccatcccccagatcgcggccaacctcgccagccgcggcctgtgcgccggcatgatctccacgtcgaccggccaggccgtcgcgccgaacgacaacccgcccaccaccggcccgacgaccaccaccacgacgacgtcccagcagcccggcggctcgtgcaccgcgacctaccggaccacccagcagtggggcgaccgcttcaacggcgaggtgaccgtccgggcgggcgcctccgcgatcaccagctggacggccaccgtcacggtgacctccccgcagaaggtgtcggccacctggaacggcacgccgagctgggactccagcggcaacgtcatgaccatgaagcccaacggcaacggaaacctcgccgcgggcgcgagcacgacgttcggcttcaccgtgatgaccaacggccagtgggcggcgccgaccgtctcctgccgcacgccgtga

SEQ ID NO: 126 - DNA, 1410 bp, Cellulomonas palmilytica
  source 1..1410: mol_type = genomic DNA
atgaccccctcatccatgtccagacgcgcgcgcttcgcggccgcgctcgcagtcgtcacgctcggggcgaccatcgctgcgacgatccccgcccaggccgcgggaagcacgctgaaggatgccgcagcgcagagcggccggtacttcggcaccgcgatcgccggcttcaagctgagcgactcgacgtactcgtccatcgcgaaccgtgagttcaacatgatcacggccgagaacgagatgaagatggacgcgacggagccgtcgcagaacaacttcaacttctccagcggcgaccagatcctcaactgggccgtccagaacggcaagcgcgtgcgcggccacgccctggcctggcactcgcagcagcccagctggatgcagggcatgtccggcagcgcgctgcgcagcgccatgctcaaccacgtcaccaaggtcgccgagcactacaagggcaaggtctatgcctgggacgtcgtgaacgaggcgttcgacgacagcaacggcgggcgccgcgactccaacctgcagcgcaccggcaacgactggatcgaggccgcgttcaaggccgcccgcgccgccgacccgaacgccaagctctgctacaacgactacaacaccgacaactggacctgggccaagacgcagggcgtctacaacatggtcaaggacttcaaggcccgtggcgtgccgatcgactgcgtcggcttccagtcgcacttcaacgcgcagagcgcctacaacagcaactaccgcaccacgctgtcgagcttcgcggcgctgggtgtcgaggtccagatcaccgagctcgacatcgagggctccggctcgcagcaggcggacacgtaccgtcgcgtcgtcgaggactgcctcgccgtcaaggcctgcaccggcatcacggtgtggggcgttcgtgactccgactcgtggcgctcctacggcacgccgctgctgttcgacaacaacggcggcaagaaggccgcgtacacctcggtcctcaacgcgctgaacgccgcggacccgacggacccgacgaccgaccccacgcaggacccgacggacgacccgacccaggacccgacggacgacccgacggacgacccgacggacccggagacgacgaacccgccggacccgaccggtaagtgctccgcggcgctcacgatcgtgaactcctggccgggcggctaccaggccacggtgacggtcaaggccggctcctcgtcgatcaacggctggcgggtcaccctgcccagcagtgtgaacacgaacaacctgtggaacggcgtcctgtccggtggcgtggtgaccaacgccccgtacaacggctcggtcggtgccggccagtcgacgaccttcgggttcgtcggcaacggcagcgccccgggcgcaggcaacctgacctgcgcctga

SEQ ID NO: 127 - DNA, 1374 bp, Micromonospora carbonacea
  source 1..1374: mol_type = genomic DNA
atgagacgcaagagagccctcctgacgacggtgaccctcgcggtcaccggcgcactcaccgccggcgtgctggtgacgatggcccccgccgccagcgccgggacgaccctgcgggctgccgcggccgagaagggccgctacttcggcgccgcggtcgcgacgggcaaactctccaacagcacgtacacgacgattctcaaccgcgagttcaacagcgtcgtggccgagaacgagatgaagtgggacgccaccgagccgcagcagggccagttcaactacagcggcggcgaccgcctcgtcagccacgcccgggccaacgggatgagcgtgcggggccacgccctgctctggcaccagcaggagccgggctgggcgcagggcatgtccggcagcgccctgcgcagcgcgatgatcaaccacgtcacccaggtcgccacccacttcaaggggcagatctacgcctgggacgtggtgaacgaggcgttcgccgacggcaacagcggcggccggcgtgactcgaacctccagcgcaccggcaacgactggatcgaggcggcgttccgcgccgcgcgggccgccgacccgggcgcgaagctctgctacaacgactacaacaccgacggggtcaacgcgaagtcgaccggcatctacaacatggtgcgcgacttcaagtcccggggcgtgccgatcgactgcgtcggcttccagtcccacctgggcaccacgctggccagcgactaccaggccaacctccagcgcttcgccgacctcggcgtcgacgtgcagatcaccgagctggacgtcatgaccggcggcaaccaggcgaacatcttcggcgcggtgacccgggcgtgcatgaacgtgtcgcgctgcaccggcatcacggtgtggggcgtgcgggactgcgactcgtggcgggggtccgacaacgccctgctgttcgactgcaacggcaacaagaagccggcgtacgactccgtcctcaacgccctcaatgccggcaccggcatccccaacccgacgaccaccccgccgaaccccaccaccactccgccgaacccgacgactccgccgccgggtggggccgggtgttcggcgacggtgtcggcgaattcgtggacgggtggtttcgtggccacggtgaaggtgaccgctggttctggtggtacccggggttggaacgtgagtgtgacgttgccgggtggtacgagtgtcacgggtacgtggtcggcgacggccagtggtagttcggggacggtgcggttcgccaacgtggactacaacggtcagctcgccgccggtcaggtgaccgagttcgggttccagggcaacggcaccgcgcccacccagacccccacctgcaccgccagctga

SEQ ID NO: 128 - protein, 469 aa, Cellulomonas palmilytica
MTPSSMSRRARFAAALAVVTLGATIAATIPAQAAGSTLKDAAAQSGRYFGTAIAGFKLSDSTYSSIANREFNMITAENEMKMDATEPSQNNFNFSSGDQILNWAVQNGKRVRGHALAWHSQQPSWMQGMSGSALRSAMLNHVTKVAEHYKGKVYAWDVVNEAFDDSNGGRRDSNLQRTGNDWIEAAFKAARAADPNAKLCYNDYNTDNWTWAKTQGVYNMVKDFKARGVPIDCVGFQSHFNAQSAYNSNYRTTLSSFAALGVEVQITELDIEGSGSQQADTYRRVVEDCLAVKACTGITVWGVRDSDSWRSYGTPLLFDNNGGKKAAYTSVLNALNAADPTDPTTDPTQDPTDDPTQDPTDDPTDDPTDPETTNPPDPTGKCSAALTIVNSWPGGYQATVTVKAGSSSINGWRVTLPSSVNTNNLWNGVLSGGVVTNAPYNGSVGAGQSTTFGFVGNGSAPGAGNLTCA

SEQ ID NO: 129 - protein, 437 aa, Promicromonospora iranensis
MTRTLTSPRHRRSLRAISLATLTAVVLAGGMAATTAQAAGSTLQAAATEKGRYFGTAIAANKLSDSTYTTIANREFNMVTAENEMKIDALEPNQNQFNWTNGDRIVSWARSNGKQVRGHTLAWHSQQPGWMQNMSGTALRNAMLNHVTQVATHYRGQIHSWDVVNEAFQDGSSGARRDSNLQRTGNDWIEAAFRAARAADPNAKLCYNDYNTDDWTHAKTQAVYNLVRDFKARGVPIDCVGLQSHFNAQSPVPSNYQTTISSFAALGVDVQITELDIEGSGSAQADNYRKVVQACLAVSRCTGISVWGVRDTDSWRASGTPLLFDGNGNKKPAYTATLDTLNGGTSNPQPGGCTVAVTRSTDWSDRFNVSLAVSGSSTWRVSIQLQGGQTLQNSWNATVSGSSGTLTATPNGAGNNFGITVYKNGNTNLPTATCSTT

SEQ ID NO: 130 - protein, 480 aa, Micromonospora ferruginea
MDKVFARTSHSTAARPRTRAAVVSMLAGAAAVAATVAVATSASAGTTLGASAAEQGRYFGTAVAANKLSDATYVGILNREFNMVTPENEMKWDATEPSQNQFTYSSSDRIVAHAQANGMRVRGHALAWHSQQPGWAQSLSGTALRNAMVNHITQVATHFKGKIYAWDVVNEAFDDGNGGRRDSNLQRTGNDWIEVAFRTARAADPGAKLCYNDYNTDNWTWAKTQGVYNMVKDFKARGVPIDCVGFQSHFNSGSPYPGNYRTTLQNFADLGVDVQITELDIEGSGSAQATTYGNVVKDCLAVARCNGITVWGIRDSDSWRASGTPLLFDGSGNKKAAYTSTLNALNAGGTTPPTSTPPTSTPPTSTPPTTPPPTTTPPTTPPPAGACTASLTTNQWQGGFVTTVRVTAGGSALNGWSVSLTLPSGSSVTNTWSAQASGAGGAVTFRNVDYNRQVGAGGSTEFGFQGTGTAPSGTVSCTAG

SEQ ID NO: 131 - protein, 444 aa, Actinoplanes friuliensis
MLSAASAGVVLAASIAVVGTAEAASTLGASAAQTGRYYGAAIAAGRLGDSTYTRILNTEFNSVTPENEMKWDATEPSQGRFTYTNGDRILNQGLSNGSKVRGHALLWHAQQPGWAQALSGSALRNAAINHVTQVATHYKGKIYAWDVVNEAFADGGSGGRRDSNLQRTGNDWIEAAFRAARAADPAAKLCYNDYNTDGINAKSTGIFNMVRDFKSRGVPIDCVGFQSHLGTGIDSTYQANLKRFADLGVDVQITELDIEQGGNQANIYSTVTKACLAVSRCTGITVWGIRDTDSWRTGANPLLFDGSGNKKPAYTAVLNALNAGGTTNPTTPPVTSPPASPTTPPPSGSGCTATVSLNSWSGGYVATVKVTAGSSAVTGWTVSATLPSGGALTGVWSATNTGTTGAVSFRNVEYNGRIPAGGSTEFGFQGTGTGPSAAPACRVG

SEQ ID NO: 132 - protein, 759 aa, Actinoplanes ianthinogenes
MIVAGIAGLVAAGLGVVYTQTADAATTLSASANQTGRYFGTAIPVSKLGDSAYTTILKTEFNAVTPENEMKWDATEPSQGSFNYTNGDKILNQGRAQGAKVRGHALLWHSQQPTWAQSLSGSALRTAAINHVTQVATHYKGKIYAWDVVNEAFADGGSGGRRDSNLQRTGNDWIEAAFKAARAADPAAKLCYNDYNTDGVNAKSTGVYTMVKDFKARGVPIDCVGFQSHLGTTVPADYQANLQRFADLGVDVQITELDVAQGGNQANIYASVTKACLAVSRCTGITVWGIRDSDSWRTGENPLLFDNSGTKKAAYTSVLNALNAGGTKPATSATTTPPGTAAPSAGAGCTAKLTAGEVWGDRYNSTITVSGASTWTVVVTITAPQKVSTVWNGTATYGGGSGQIMTVKPNGNGNSFGFTTMNNGNSTARPTITSCVAGGSTTSATAAPTTVPASAPSSCALPSKYRWTSTGALATPKSGWASLKDFTVVPYNGKHLVYATTHDTGSSWGSMNFSPFTNFSDMASAGQNAMSSGAVAPTLFYFAPKKIWVLTYQWGGSAFSYRTSSDPTNANGWSAAKPLFTGSISGSGTGPIDQTIIGDGTNMYLFFAGDNGKIYRASMPIGNFPGNFGSSYTTIMSDTTNNLFEAPQVYKVQGQNQYLMIVEAIGSNGRYFRSFTATSLSGSWTPQAATESNPFAGKANSGATWTNDISHGELVRVTADQTMTVDPCHLQLLYQGRSPSSGGDYGLLPYRPGVLTLQR

SEQ ID NO: 133 - protein, 475 aa, Antribacter gilvus
MKDLSPHRAGKFPWRSLAGAAGALAVAASLAVGVSTSAQAAGTTLQAAAAESGRYFGTAIAANKLSDSTYTTIANREFNMITAENEMKLDATEPSQNQFNYTNGDRIVNWATSNGKQVRGHTLAWHSQQPGWMQNMSGTALRSAMLNHVTQVATHYRGKIHSWDVVNEAFQDGSSGARRDSNLQRTGNDWIEAAFRAARAADPNAKLCYNDYNTDDWTHAKTQAVYNLVRDFKARGVPIDCVGLQSHFNSQSPVPSNYQTTLSSFAALGVDVQITELDIEGSGTAQADNYRRVVQACLAVSRCTGITVWGVRDTDSWRSSGTPLLFDGSGNKKAAYTSTLNALNAGGTTNPPTTPPPTTPPPTTPPPTTAPPTTPPPTQPGACTAAYRLVNSWQGGFQAEVVVTAGSARTGWTTSFTLPGGTSISQLWSGTLTSSGSTHTVRNVSWNGNLGAGQSTTYGFTGTGSAPSSATVSCS

SEQ ID NO: 134 - protein, 653 aa, Nonomuraea jiangxiensis
MRTNALPPPRTRRSFGGFRRAVAVGVLALAGTIAPLALSVPADAAETTLGAAAMQSNRYFGAAIAAGKLNESAYTTIANREFNMVTPENEMKIDATEPNRGQFTFTNADRIYNWAVQNGKRVRGHTLAWHSQQPGWMQSLSGSTLRQAMIDHINGVMAHYRGNIYAWDVVNEAFADGNSGGRRDSNLQRTGNDWIEVAFRTARAADPAAKLCYNDYNIENWTWAKTQGVYNMVRDFKSRGVPIDCVGLQAHFNSGSPYNSNFRTTLSSFAALGVDVQITELDIQGASATTYANVTNDCLAVPRCTGITVWGVRDSDSWRSGDTPLLFDGSGNKKPAYTSVLNALNSVNPNPDTTPPSTPGTPAASNVTSSGATLTWAASTDTGGSGLAGYNVYREQGTTDPQLGQSATNSITLTGLTAGTQYQVYVRARDGAGNLSGNSQLVTFTTQTGGGTDTTPPSTPGTPAASNVTASGATLTWTASTDTGGSGLAGYDVYREQGATDPLLGQSATNSITLTGLTAGTQYQVYVRARDGAGNLSGNSQPATFTTTGGGTGGSCTVTPTTQTQWPSGYVIDPVRVTAGTSAISGWTVTFTLPAGHTVTGSWNTQLTVSGQTVTARNAAHNGNLGPGASTAFGFQVSRPNGNTSLPSGYTCA

SEQ ID NO: 135 - protein, 634 aa, Kibdelosporangium aridum
MSRTVGRHAALLALLGTFLLPGTADAGTTLGASAAEKGRYFGAAVAAHKLGDSTYVGILNREFNMVTPENEMKIDATEPNQNQFSFGNADRIVNHAVSQGMRVRGHTLAWHSQQPGWMQNMSGSALRQAMLNHVTRVASYYRGKIYAWDVVNEAFADGSSGARRDSNLQRTGNDWIEAAFRAARAADPNAKLCYNDYNTDDWSHAKTQAVYRMVQDFKSRGVPIDCVGLQSHFNNNSPYPSNYRTTLSSFAALGVDVQITELDIEGAPPTTYGNVVRDCLAVARCNGITVWGIRDSDSWRASQTPLLFNNSGGKKPAYDAVLSALNSGSIPPPGGSNCSAGYVGLTFDDGPNSSTTPQLLNALRSAGVRATFFNVGQRVQQNPALTRSQIDAGMWVGNHSWTHPHLTQMTAAQITSELSQTQQALQQATGQTPRLFRPPYGETNSTVRQVQAQLGLTEVMWTVDSQDWNNASTAQIVQAASTLQPGGIILMHDGYQTTVNAIPQIVANLSSRNLCAGMISTNGQVVAPGTEPGNGSCTASYRTTQQWGDRFNGEVTIKAGSAAITSWTSTVTITAPQRVSTTWNGTASWDSSGTVMTMKPNGNGNLAAGASTTFGFTVMANGQWAQPSVSCGSP

SEQ ID NO: 136 - protein, 472 aa, Actinomadura madurae
MRANVIPKSRIRIRKRAILAGALGVLATAALVAPSPAAAAESTLGAAAAQNGRYFGTAIASGKLNDSVYTTIANREFNSVTAENEMKIDATEPQRGRFDFSAGDRVYNWAVQNGKQVRGHTLAWHSQQPGWMQSLEGGTLRQAMIDHINGVMAHYTGKIVQWDVVNEAFADGSSGARRDSNLQRTGNDWIEVAFRTARAADPAAKLCYNDYNIENWTWAKTQAVYSMVRDFKQRGVPIDCVGFQSHFNSGSPYNGNFRTTLQSFAALGVDVAITELDIQGASATTYANVTNDCLAVSRCLGITVWGVRDTDSWRSEQTPLLFDGNGNKKAAYTAVLNALNGGTTTPPDGAGTIKGDGSGRCLDVPNASTTDGTQVQLWDCHGGTNQQWTYTDASELRVYGDKCLDAAGTGNGARVQIYSCWGGDNQKWRLNSDGSVVGVQSGLCLDAAGTANGSAIQLYSCWNGGNQRWTRT

SEQ ID NO: 137 - protein, 484 aa, Nonomuraea indica
MRVDAMSPPTARRPLGARRRTLITGVLGALGLITALAPAIPADAAASTLGAAAAQSGRYFGTAIASGKLGDSAYTTIAGREFDMVTAENEMKIDATEPNRGQFTFTAGDRVYNWAVQNGKRVRGHTLAWHNQQPGWMQSLSGSSLRQAMINHINGVMGHYKGKIYAWDVVNEAYADGNSGGRRDSNLQRTGNDWIEVAFRTARAADPAAKLCYNDYNIDNWTWAKTQGVYNMVRDFRARGVPIDCVGLQSHFNANSPYNSNYRTTIQSFAALGVDVQITELDIQGGSATTYANVVRDCLAVPRCTGISVWGVRDSDSWLGAGATPLLFDGNGNKKAAYTAVLDVLNGGSTTPPPGDAGQIRNTASGRCVDVPNSATADGTAVQLWDCNGQSNQRWTRTAAGELKYGDKCLDAGGTGNGARIQIYSCWGGDNQKWRLNSDGTIVGVQSGLCLDAVGGGTGNGTGLQLYGCWGGGNQRWDYNPGTA

SEQ ID NO: 138 - protein, 680 aa, Saccharothrix syringae
MSRTAVTAAGRSEARGRSRRAAVVVGAIGLLSAAAVVLPNVATAGTTLGASAAESGRYFGTAVAANKLSDSTYVGILNREFDMVTAENEMKMDATEPNQNQFSFGNGDRIVNHARNQGKRVRGHALAWHSQQPGWMQNMSGTALRNAMLNHVTQVATYYKGKIYAWDVVNEAYADGSSGGRRDSNLQRTGNDWIEAAFRAARAADPNAKLCYNDYNTDNWSHAKTQGVYRMVQDFKSRGVPIDCVGFQAHFNSGNPVPSNYHTTLQNFADLGVDVQITELDIEGSGSTQAQQYQGVVQACLAVTRCTGITVWGIRDSDSWRSSGTPLLFDGSGNKKAAYTSVLNALNAGSTAPPSTTTTTTPPNTSDCRAGYVGLTYDDGPNGSTTTQLLNALRSAGLRATFFNQGNRVQQNPGLAKAQRDAGMWVGNHSWSHPHMTQLSQSQMASEISQTQQAIQSATGEAPKLFRPPYGETNSTLKSVEAQYGLTEVLWSVDSQDWNNASTAQIVQAASTLQNGGVILMHDGYQTTINAIPQIAANLASRGLCAGMISTSTGQAVAPNDNPPTTGPTTTTTTTSQQPGGSCTATYRTTQQWGDRFNGEVTVRAGASAITSWTATVTVTSPQKVSATWNGTPSWDSSGNVMTMKPNGNGNLAAGASTTFGFTVMTNGQWAAPTVSCRTP

SEQ ID NO: 139 - protein, 471 aa, Cellulomonas cellasea
MNRSGLRPPSSTHRRRTGAVLVAALAVTLAGATLPAQGAGSTLQAAAAESGRYYGTAIAANKLSDSTYTTIANREFNMITAENEMKMDATEPSQNQFNYSSGDRIVSWARSNGKRVRGHALAWHSQQPGWMQNMSGTALRNAMLNHVTQVATHYKGQIYAWDVVNEAYADGSSGARRDSNLQRTGNDWIEAAFRAARAADPAAKLCYNDYNTDNWSHAKTQGVYTMVRDFKSRGVPIDCVGFQAHFNSGNPVPSNYDVTLRNFAALGVDVQITELDIEGSGSSQAQQYAGVHQACLSVARCTGVTVWGVRDTDSWRASGTPLLFDGSGNKKAAYTSTLNALNAGGTTTPNPTPNPTTPQPTPTTTTPPVTGTGSCTATYSEGQKWGDRFNGVVTIRANSAITSWTSTVTVSQAQRITSTWSGTPSWDSSGKVMTMRPAGNGTLAAGQTTSFGFTVLHGGDWTWPRVTCSAS

SEQ ID NO: 140 - protein, 457 aa, Couchioplanes caeruleus
MHKPRTPLRALGVAAAAAVVAAGSVVALTSTAEAATTLGASAAATGRYFGTAVAANKLSDSTYVGILDREFNAVTPENEMKWDATEPNQNQFNYSSADRIVSHAQAQNMRIRGHALAWHQQQPGWAQNLSGTALRNAMLNHVTTVAAHYKGQIYAWDVVNEAFDDGGNGARRDSNLQRTGNDWIEAAFRAARAADPGAKLCYNDYNTDGQNAKSNAVYAMVQDFKSRGVPIDCVGFQSHFNAQSPVPSDYQANLQRFANLGVDVQITELDIEGSGQTQADNFGRVVKACLAVSRCTGITVWGIRDSDSWRASGTPLLFDSSGNKKAAYTSTLNALNSGGTTPPSDPPTSPSTPPSTPTDPGTGACTATISLNQWNGGFVATVRVSAGSSALSGWSVSTTLPSGAAVTNSWSSQSSGSSGTVRFANVSYNGSVAAGGSTEFGFQGTGTGPSSATCSAS

SEQ ID NO: 141 - protein, 473 aa, Glycomyces tritici
MITSTPRRRRRVWSTIAAVATASLAATTALVMLPGTAQAGTTLGASAAESGRYFGAAVSTQYLSESAYANTLGAEFNSVVAENAMKWDATEPNPGQFNFSGGDQLVNWAQARGMKVRGHTLVWHAQQPGWAQNLTGQNLRNAMLNHIAGVANHYEGDVFAWDVVNEAFEWDGSRRQSNLQRQLGNGWIEEAFRAADAADPTATLCYNDYGTDSINAKSTAIYNMVRDFKSRGVPIDCVGFQTHIAHNENLSSYQANLQRFADLGVDVEITELDVGQGSGQAATYGTVTNACMAVARCKGITVWGITDKYSWRDDNPLLFDGNYQKKQAYHTVLAALNNGNPGGGGGKHITSAASGRCLDVPNQSTTNGTQLQIYDCWTGANQQWTVANGEISVYSGGSKKCLDASGGGTANGTAAIIWSCHGGANQRWNVNSNGTITNAASGLCLDVAAAATANGSKVQLWSCTGGSNQRWTV

SEQ ID NO: 142 - protein, 475 aa, Actinoplanes auranticolor
MTVIAARARLLLGAACAVVIAVSSVAVATLAHADLTGPPGTTLKAAAERSGRYFGAAMGRDRLTDNGFLTIANREFDMMTAVNEMKPDATEPNRGQFDFRAGDAIYNWATQRGMRFRGHTLAWHAQQPRFWGSLSGSALRQAMIDHINGVMAHYKGKLYAWDVVNEAFAENGSRRSSNLQATGNDWIEAAFRAARAADPGVQLCYNDYNIENWTYAKTQGVYNMIRDFKARGVPIDCVGLQTHFTGGSSLPGNFQQTLSSFAALGVDVALTEADVTNASSSQYQGLTQACMNVPRCVGITTWGIRDSDSWRGNENPLLFDRNSNPKPAYTSVLNALNAASTTVPGTSTPPSTPPSTPPSTPPSTPPSTPPVTGQPGACTATYRTASTWNGGYQGEVTVANNGSAALTGWTVQLTLAGGQTVANVWNGINTGTSGTISVRNAAYNGSVGANASTSFGFLVNGSTGTAPGSLTCTSP

SEQ ID NO: 143 - protein, 456 aa, Sphaerisporangium rubeum
MVVIGTAVVVGAGVCAGAGAADAAGTLGAAAAGSGRDFGTAVQAGRLGEAAYVETLDREFTSVTPENEMKWDAVEPARGTFVFTAADRIVEHARARGMKIRGHTLVWHPGLPGWLTNLSSAEFRIAVNNHIATVMGHWRGQIDSWDVVNEAFQDGSGALRSTIFRSRLGDGYIEEAFRAARAADPAAKLCYNDYNIEDADAAKTRAVYAMVRDFKERGVPIDCVGVQSHFNASMPFPANYRRTIEQFAALGVDVQITQLDVDGGAAQAATYRAAVEACVAVPRCTGITVWGVPDHYSWRAPNTPLLFDRDYQKKPAYFAVLEALNAAGGEPTPAPTPTPSPTPTPTPTVTPVPVSCRVTAELWARSRTGYVIKPVTVRNTGRAAVSGWTVTFTLPQGHVVTGSWDAVLTVAGGTVTARNAGHNGTLAPGATATFGFQVRRASGGTALPSGYACRAG

SEQ ID NO: 144 - protein, 557 aa, Phytoactinopolyspora halotolerans
MITRHRRQAILASMALVGATLVVPIGAAQAQDTLRGAADAQGLRMGAAVANGPLSSDAQYRNILGTEFNSVTAENSMKWESLQPSQGQFTFSQGDAIVDFAQANNQAVYGHTLVWHNQTPSWVQNLSGQQLDDAMQTHITTVLDHYEGQVEAWDVANEVIDDGANLRNSFWLQGLGEGYIADAFRYADAADPNAKLYINDYNIDGINAKSNAYYDLVSGLLNQGVPIDGIGLQAHMILGQVPSSLEDNIRRFAQLGLEVRITELDIRMDLPVTQAKLEQQRQDYAAVVDACMSVDGCVGVTTWGFTDAHSWVPDQFGGQGAALPFDENYNKKPAYYGILDTLDGGTPNDTTPPTQPGAPQISDVTSNSASLTWSASSDSGGSGLAGYSVYREQSGNDQLLASPSTNSVTLTGLDPQTQYSVYVVARDGAGNTSSPSTAASFTTQEGPGGGGACDVEYTANNWGGSEGFTASVTITNTGSSQLNGWTLGFTFPGDQSVREGWSATWSQSGADVTAESIGWNDALAPGGSTTVGFNGTYSGSNPEPTEFTLNGEPCSVS

SEQ ID NO: 145 - protein, 466 aa, Glycomyces paridis
MHTRKRRRALIAGVVTVATAALGIGLSTQFASAQTTLRGAADEAGIDIGIAVDANQLQNNATYRNLVATEFNSLTAENAMKWDATEPSDNSWNFSGADAIVDFAEDNDQTVHGHTFVWHSQTPQWVQNLSASAMQAAMVDHINTLANRYEGRVDSWDVVNEVVSDNNGAMRDSFWRNTLGDGYITTAFQTARAADPNADLYINDYSIEGDNAKSDRIYTIAQGLNSQNLIDGVGFQSHLILGQVPSTMEANLQRFIDLGLKVRITELDIRIQTPADANELQQQANDYERVVSICAELADCSGVTVWGLRDADSWVPGVFPGYGAPLLFDDNFGKKPAYNAVLEALGGDGSEEPTTTDPGTTTPPPTGDGDCTAEIDVVNDWGSGWQGNVLITADNGAVNGWTLTWTWPSGQSITSSWNATVTSSGSSVTASDVGWNANIAAGQTMNAWGFVGSGSSAAPQVTCTAD

SEQ ID NO: 146 - protein, 486 aa, Streptomonospora alba
MRLVRHGGHETPRRTRAGSARRVAGAALATFLGAAVLAPAGPAAADSPLRDHAAQQGFEIGAALGVDHLQNDSQFADLAATEFNAATAENSMKWESTEPSRGQFDFSGADAFMDFAQQNNQKVRGHTLVWHSQLPSWVENGNFSQSELRSVMEDHIDEVAGRYAGEVAYWDVANEIFEGDGSWRNSVFYDTLGPDFVADALRMTAEADPNAELWLNDYSIDGINAKSDAYYNLIQDLQAQGVPIDGIGLQAHLINGQVPGDLQQNIQRFADLGIKVAITELDVRIDMPASQSELQQQAQDYRAVMDACLAVNGCVGVTVWGIVDKYSWVPDTFEGEGAPLLFNDDYQPKPAYDAVHEALGGSSDDDDDDDNGGGEDPPPTGPCEVSYSVANEWNSGFTGQVTVTNGGSSALNGWDLQFDFSGGQQITNGWNADWSQSGSTVTASNTEWNGSVPAGGSVDIGFNASHSGSNPEPGSFALNGESCSVS

SEQ ID NO: 147 - DNA, 1413 bp, Synthetic construct
  source 1..1413: mol_type = other DNA
atgacgccaagttcaatgtcccgtcgggcccgcgttgcgtcagctcttgccatcgtgacgttgggcgctactatcgctactacgattcctgcccaagccgccggctctacattacaagcggctgcttctgaaagtggtcgttatttcggtacagcgattgctgcgtttaaattgaatgattcaacgtacagctccattgcgaatcgggagttcaatatgattacagcagaaaatgaaatgaaaatggacgcgactgagccgtctcagaataatttttcgtattcgagtggcgatcagattcttaattgggcacggtctaatggtaagcgtgttcgtggccatgcattggcctggcattcgcagcaaccggggtggatgcaaaatatgtccggtacacagttacgcaatgccatgttgaatcatgtgacgcaggttgctacgcattataaaggcaagatttatgcatgggatgtcgttaatgaagcctatgcggactctggtggggggcgtcgtgacagtaatcttcaacggaccggtgatgattggattgaagctgcttttcgcgctgcgcgggcggcagatccaggtgccaaactttgctataatgattataatacagacaattggacgtgggctaaaacacagggtgtctacaacatggttaaagattttaaagctcgtggggtgccgattgattgcgttggctttcaatctcatttcaattccgggtcgccgtatccgtcaaattatcgtacaaccttacaaaatttcgccgccctgggtgtggaagttcagattaccgaattagatatcgaaggttcggggcaacaacaggcacagacttatgccaatgtcgtcgctgattgcttggcagtgaaggcttgcaccggtatcacagtttggggcgtcagagactcagactcttggcgcagctccgggacgccgttgttgtttgatggcagcggtaataaaaaggctgcgtatacctctaccttggacgctcttaaccggggtggagtcccgactgatccgaccactccgccgacagaccccacgacaccacccactgatcccacgacaccgcctaccgatcctaccactccgcctacggatcctacaaccccacccacggatccaactggccgttgcaccgcgagccttgcgattgccaacgcctggccgggtggatatcaggccacggtcacggtcaaagcagggtccagtagtatcaatggatggcgcgttacattaccttctggcgtttccactagtaacctgtggaatggcgtcctggcgaacggagtggtcacgaatgccccttataatggctctgttggcgcggggcaatccacgacctttggctttgtcggcaatggctcggcgcccagtgctggtagcgtgacttgtgcctga